(19) United States
(12) Patent Application Publication
(10) Pub. No.: US 2017/0342084 A1
(43) Pub. Date: Nov. 30, 2017

(54) REDUCTION OF ALPHA, BETA-UNSATURATED KETONE LEVELS IN MORPHINAN DERIVATIVE COMPOSITIONS

(71) Applicant: Rhodes Technologies, Coventry, RI (US)

(72) Inventors: Keith Edward MCCARTHY, Old Lyme, CT (US); Helge Alfred REISCH, Westerly, RI (US); Christopher SPROUT, Riverside, RI (US)

(21) Appl. No.: 15/324,528

(22) PCT Filed: Jul. 8, 2015
(86) PCT No.: PCT/IB2015/055171

§ 371 Date: Jan. 6, 2017

Related U.S. Application Data
(60) Provisional application No. 62/022,514, filed on Jul. 9, 2014.

(51) Int. Cl. C07D 489/08

(52) U.S. Cl. CPC C07D 489/08; C07B 2200/13

(57) ABSTRACT

The disclosure relates to processes for reducing the amount of a compound of formula (I) or a salt or a solvate thereof present in a composition comprising compounds of formulae (I) and (II) or a salt or a solvate thereof.

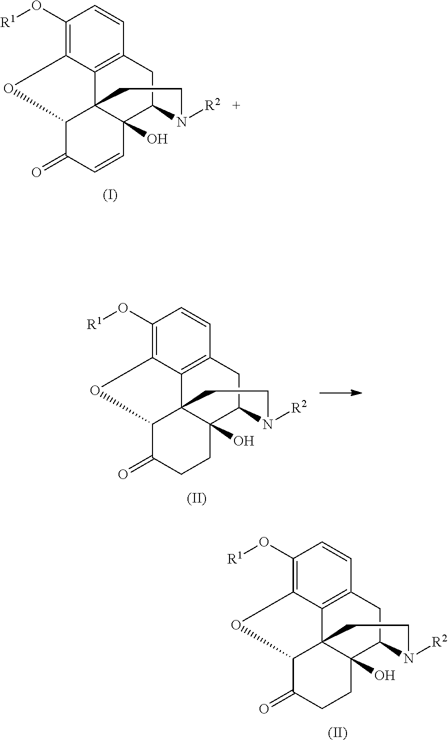

### FIELD

The disclosure is in the field of pharmaceutical compositions comprising morphinan derivatives and in the field of pharmaceutical morphinan derivative synthesis. Processes for reducing the amount of α,β-unsaturated ketone by-products in morphinan derivative compositions are provided. Also provided are morphinan derivative compositions with a reduced amount of these by-products. Morphinan derivative compositions include those containing, e.g., at least one of noroxymorphone free base and a noroxymorphone salt. The compositions can be used as starting materials or as intermediate materials in the preparation of morphinan derivatives, where the morphinan derivatives comprise, e.g., at least one of naloxone free base, a naloxone salt, naltrexone free base, and a naltrexone salt.

### BACKGROUND

Morphinan derivatives like oxymorphone, naloxone, naltrexone and their salts, e.g., their hydrochloride salts, have long been used as active pharmaceutical ingredients for different medical indications. However, several challenges remain in order to synthesize morphinan derivatives in high purity with a minimum amount of by-products.

Typically, naloxone hydrochloride (1) is synthesized in a multiple-stage process, for example, in a four-stage process, which includes oxymorphone (2) and noroxymorphone (3) as intermediate materials. A conventional route for the preparation of naloxone hydrochloride (1) from oripavine (4) is illustrated in Scheme 1 below. In a first Stage, oxymorphone (2) is prepared by oxidation of oripavine (4) to 14-hydroxymorphinone (5) which, conveniently, is not necessarily isolated. Next, 14-hydroxymorphinone (5) is reduced to oxymorphone (2). In a second Stage, oxymorphone (2) is dealkylated to yield noroxymorphone (3) which, in a third Stage of the process, can be alkylated, e.g., to naloxone (6) or to naltrexone (7). If desired, in a fourth Stage a salt, e.g., a hydrochloride salt as shown in Schemes 1 and 2, is typically obtained by the reaction of naloxone (6) or naltrexone (7) with hydrochloric acid or hydrogen chloride.

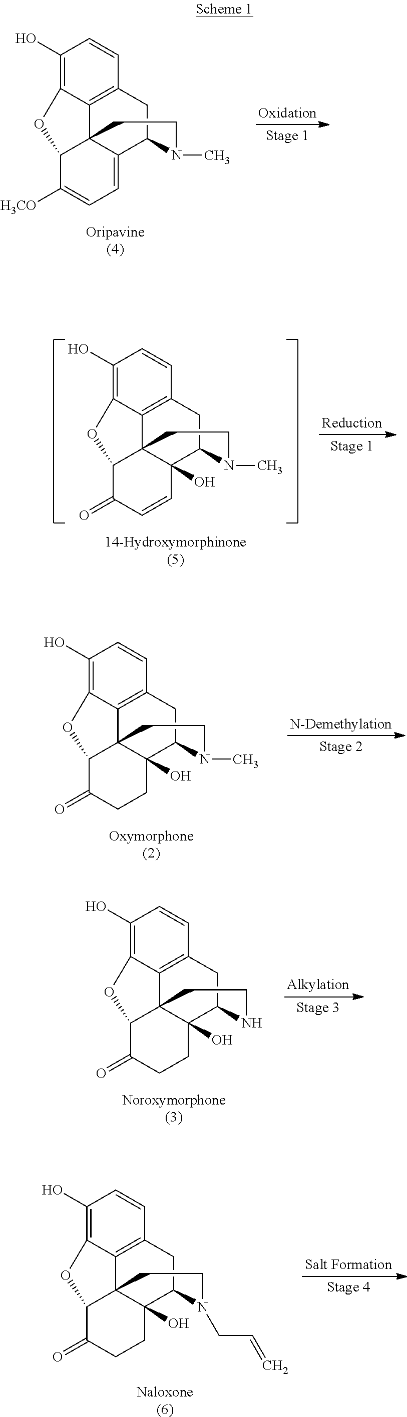

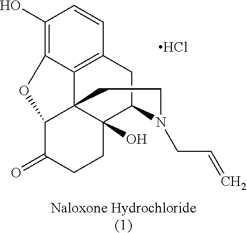 [SMILES: C=CCN1CC[C@]23c4c5ccc(O)c4O[C@H]2C(=O)CC[C@@]3(O)[C@H]1C5 ; Cl ; "Naloxone Hydrochloride(1)"]

A corresponding route for the preparation of naltrexone hydrochloride (lb) from noroxymorphone (3) is illustrated in Scheme 2 below in which Stages 1 and 2, being identical to those stages in Scheme 1, are omitted.

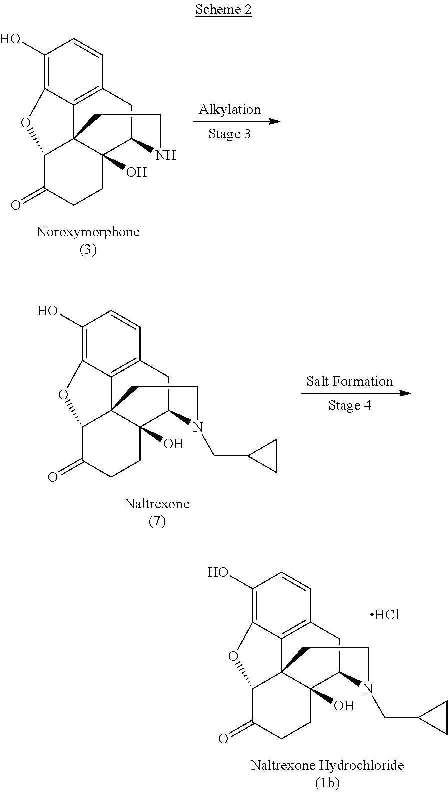 [SMILES: O=C1CC[C@@]2(O)[C@H]3Cc4ccc(O)c5c4[C@@]2(CCN3)[C@H]1O5 ; O=C1CC[C@@]2(O)[C@H]3Cc4ccc(O)c5c4[C@@]2(CCN3CC2CC2)[C@H]1O5 ; O=C1CC[C@@]2(O)[C@H]3Cc4ccc(O)c5c4[C@@]2(CCN3CC2CC2)[C@H]1O5 ; Cl]

The preparation of oxymorphone in the synthetic process illustrated in Scheme 1 encompasses 14-hydroxymorphinone as an intermediate material; however, it may not be completely converted into oxymorphone during the first Stage of the process. 14-Hydroxymorphinone belongs to the compound class known as alpha, beta-unsaturated ketones (“ABUK”s). ABUKs contain a substructural moiety (the α,β-unsaturated ketone moiety) which generates a structure-activity relationship alert for genotoxicity; therefore, ABUKs are considered to be potential genotoxic by-products. Since 14-hydroxymorphinone (5), as well as the related downstream by-products 14-hydroxynormorphinone and 7,8-didehydronaloxone, are ABUKs, regulatory authorities will not approve a pharmaceutical composition or dosage form for sale to and use by the public if the amount of ABUKs in the pharmaceutical composition or dosage form exceeds the amount set by these authorities. The European Medicines Agency (“EMA”) has currently defined the ABUK limit for ABUK 7,8-didehydronaloxone in a naloxone hydrochloride API to be not more than (“NMT”) 75 ppm relative to the quantity of naloxone hydrochloride. The United States Food and Drug Administration (“FDA”) also sets requirements that limit the level of ABUKs in morphinans, such as naloxone hydrochloride. Furthermore, it is thought that future regulation will reduce the ABUK content in naloxone hydrochloride to NMT 35 ppm. The amount of ABUKs in naloxone hydrochloride obtained via a conventional reaction of oripavine to naloxone hydrochloride, however, typically exceeds the above-stated limits.

After oxidizing oripavine to 14-hydroxymorphinone and then reducing 14-hydroxymorphinone to oxymorphone, any remaining 14-hydroxymorphinone intermediate, or reaction by-products, can be converted into various other reaction by-products in the further synthetic process, e.g., during the reaction Stages 2 to 4 shown in Scheme 1, or can be carried over into the final morphinan derivative compound, final pharmaceutical composition or final dosage form containing, e.g., naloxone hydrochloride or naltrexone hydrochloride. These by-products can be undesired in the final pharmaceutical composition or final dosage form. Separation of these by-products from the desired final opioid can be difficult, time-consuming and not cost and volume efficient.

For example, after the oxidation of oripavine and reduction to oxymorphone, any remaining 14-hydroxymorphinone (5) can be dealkylated to 14-hydroxynormorphinone (designated as “Impurity 1”), which can be further alkylated, e.g., to 7,8-didehydronaloxone (designated as “Impurity 4”) or 7,8-didehydronaltrexone (designated as “Impurity 6”) in subsequent reaction steps, and which in turn can be further converted into the corresponding hydrochloride salt. These corresponding ABUK derivatives are depicted in Scheme 3 below.

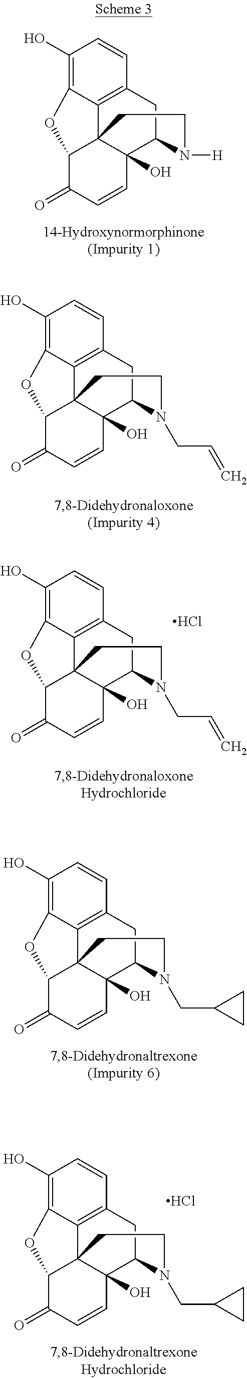

ABUKs can be very difficult to remove from the respective morphinan derivative by means of conventional purification and, as described above, only very low amounts of ABUK are considered acceptable by regulatory authorities. Conventional methods for removing by-products may not be suitable since such treatments can lead to the formation of additional undesired by-products (e.g., formed by ring-opening of the 4,5 epoxy-bridge of a desired morphinan derivative) that also need to be removed and thus further lower the yield of the desired morphinan derivative.

For example, respective ABUKs cannot easily be removed from the morphinan derivative naloxone by reduction since the allyl group of naloxone would also be reduced, resulting in additional undesired by-products.

Another option, such as the use of a scavenger to remove an impurity or impurities, is also not desirable as a final reaction step. U.S. Pat. No. 7,875,623 discloses the removal of an electrophile compound, such as 14-hydroxycodeinone, from oxymorphone (an intermediate in Scheme 1 here) using a thiol-containing compound.

U.S. Pat. No. 8,822,687 discloses a process for reducing the amount of the ABUK 14-hydroxycodeinone in an oxycodone hydrochloride preparation.

The hydrogenation of 14-hydroxymorphinone, protected with protecting groups such as carbamate, to oxymorphone has been disclosed; however, this is typically not desirable. In addition, such methods are also considered to be disadvantageous due to the conversion of 8α-hydroxymorphone (also known as 8alpha-hydroxymorphone) to an ABUK during protecting group removal, such as carbarmate hydrolysis, etc. Hydrogenating compounds bearing protecting groups has been disclosed in U.S. Pat. No. 8,227,609.

U.S. Pat. No. 7,939,543 discloses a process for reducing the amount of ABUK in an opioid analgesic composition comprising hydrogenation with diimide or a diimide progenitor.

During the oxidation and reduction reactions converting oripavine to oxymorphone, certain reaction by-products can be formed, specifically the two epimers 8α-hydroxynoroxymorphone and 8β-hydroxynoroxymorphone (also known as 8beta-hydroxynoroxymorphone). It is thought that the hydroxyl-group at position 8 in the 8α-epimer can potentially dehydrate, resulting in the formation of additional ABUKs. However, it is believed that the hydroxyl-group of the 8β-epimer may not as easily be dehydrated to an ABUK due to steric hindrance.

During the synthesis of naloxone it has been found that the predominant portion of 8-hydroxynoroxymorphone impurity obtained is 8β-hydroxynoroxymorphone. It is thought that the α-epimer is presumably dehydrated during the acidic carbamate hydrolysis step of the naloxone synthesis. The conversion reaction of 14-hydroxynormorphinone (Impurity 1) into 8-hydroxynoroxymorphone is illustrated in Scheme 4 below.

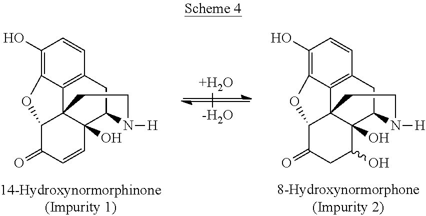

After the oxidation and reduction steps converting oripavine to oxymorphone, any remaining 8-hydroxynoroxymorphone, in the form of the 8α- or 8β-epimer, can be converted to further reaction by-products, e.g., it can be dealkylated to α- and β-epimers of hydroxynoroxymorphone and subsequently alkylated, e.g., to α- and β-epimers of 8-hydroxynaloxone and their corresponding hydrochloride salts. The corresponding 8-hydroxy derivatives are depicted in Scheme 5 below.

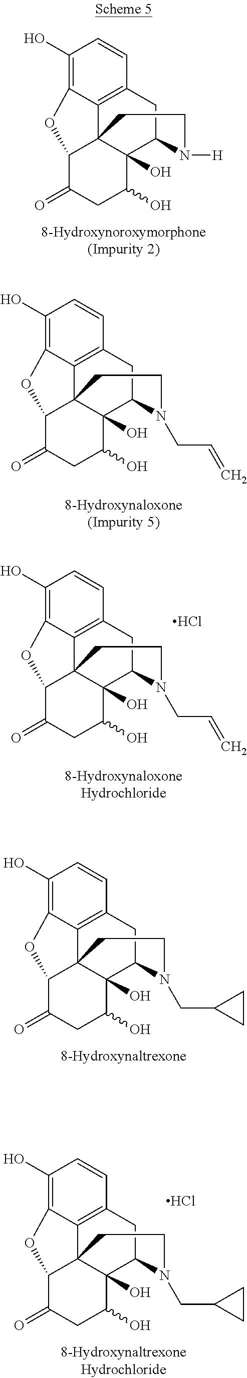

International Patent Publication No. WO 2015/015147 discloses a process for reducing the amount of ABUK present in a morphinan-6-one compound which intentionally includes, along with the starting morphinan-6-one compound, the 8-hydroxy derivative thereof.

Furthermore, chloroformates can be used in the demethylation of morphinan derivative compounds, such as in the demethylation of oxymorphone to noroxymorphone as described in Stage 2 of Scheme 1. The carbamate intermediate in the reaction needs to be hydrolyzed under harsh conditions, such as at high temperature and under acidic conditions, which can lead to the formation of additional colored by-products. Furthermore, it is thought that these harsh conditions also contribute to the conversion of the 8α-hydroxynoroxymorphone to ABUK.

Thus, there is a continuing need for high-purity morphinan derivative compositions, such as compositions of noroxymorphone, naloxone, naltrexone and salts thereof, exhibiting reduced levels of related by-products and, in particular ABUKs, which are below the thresholds set by regulatory authorities.

There is also a continuing need for processes for preparing morphinan derivatives that exhibit a reduced amount of by-products in the process intermediates (e.g., of 14-hydroxymorphinone in noroxymorphone) and/or in the final morphinan derivative product (e.g., of 14-hydroxymorphinone in naloxone hydrochloride).

There is also a particular need for having a reduced amount of by-products in intermediates if the by-products cannot easily and/or cost effectively be removed at a later point of the synthesis or from the final product (e.g., ABUK impurities in naloxone as described above).

### SUMMARY OF THE DISCLOSURE

The disclosure is directed to processes for reducing the amount of ABUK impurities in morphinan derivative compositions comprising compounds of formulae (I) and (II) or salts or solvates thereof; morphinan derivative compositions with a reduced amount of said by-products, where said morphinan derivative compositions may include, e.g., noroxymorphone base, noroxymorphone salt, or noroxymorphone base and noroxymorphone salt; said compositions for use as starting materials or intermediate materials in the preparation of morphinan derivatives, where said morphinan derivative may include, e.g., naloxone, naltrexone or salts or solvates thereof.

The process of the disclosure can further reduce the color of the initial reaction composition comprising compounds of formulae (I) and (II) or salts or solvates thereof, that may arise due to the presence of colored by-products in the initial composition. As used herein, reference to the compounds of formulae (I), (Ia), (II), (IIa), (III), (IIIa), (IV), (IVa), (V), (VI), and the like, unless otherwise indicated, also include the salts thereof. Also, as used herein, reference to named compounds (e.g., naloxone, naltrexone, etc.), unless otherwise indicated, also include the salts thereof.

The processes of the disclosure allow for a reduction of the amount of ABUK by-products in morphinan derivative compositions. The compositions of the disclosure can be used as intermediates or starting materials for the preparation of other morphinan derivative compositions which can be used without additional ABUK purification steps prior to their incorporation into pharmaceutical dosage forms.

In one embodiment, the disclosure is directed to a process for reducing the amount of ABUK by-products in morphinan derivative compositions comprising compounds of formulae (I) and (II) or salts or solvates thereof. In a preferred embodiment, the morphinan derivative is noroxymorphone or a salt thereof.

In one embodiment, the disclosure is directed to a process of isolating noroxymorphone as anhydrous noroxymorphone or noroxymorphone dihydrate. In a preferred embodiment, the defined form of noroxymorphone is the anhydrous form.

In another embodiment, the disclosure is directed to a process for reducing the amount of ABUK by-products in morphinan derivative compositions, and the use of the resulting compositions as starting materials or intermediate materials in the preparation of naloxone, naltrexone or salts or solvates thereof. In preferred embodiments, compositions comprising 14-hydroxynormorphinone and noroxymorphone or salts or solvates thereof are used as starting material for the synthesis of naloxone or naltrexone or salts or solvates thereof.

In another embodiment, the disclosure is directed to compositions comprising 14-hydroxynormorphinone and noroxymorphone or salts or solvates thereof (e.g., 14-hydroxynormorphinone hydrogen phosphate or noroxymorphone hydrogen phosphate, respectively), which compositions are useful as starting materials or intermediate materials in the preparation of pharmaceutical compositions and dosage forms comprising naloxone or naltrexone or salts or solvates thereof.

In one embodiment, the disclosure is directed to a process for reducing the amount of a compound of formula (I) or a salt or a solvate thereof

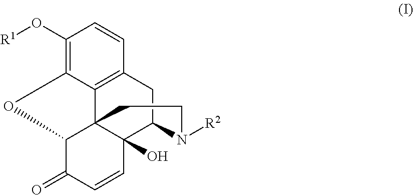

in a composition comprising compounds of formulae (I) and (II) or a salt or a solvate thereof, where the compound of formula (II) is:

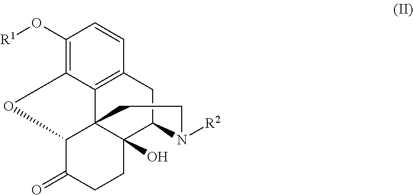

the process comprising:
(b) hydrogenating the compound of formula (I);
where:
R¹ is —H, (C₁-C₇)alkyl, or an O-protecting group; and
R² is —H, —(C₂-C₄)alkenyl, —(C₂-C₇)alkyl, —(C₁-C₇)alkyl-(C₃-C₇)cycloalkyl, —CN, —C(═O)O—(C₁-C₆)alkyl, —C(═O)O-phenyl or an N-protecting group.

The composition comprising compounds of formulae (I) and (II) contains compounds of formulae (I) and (II), which are depicted below:

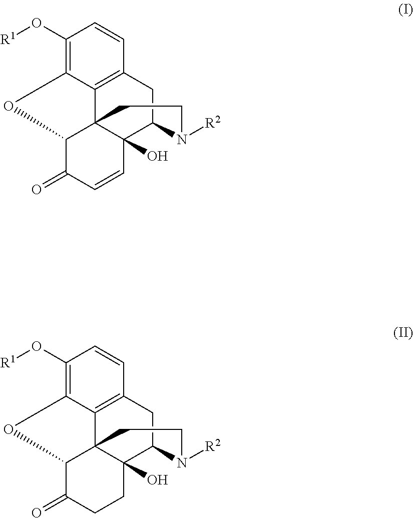

where:
R¹ is —H, (C₁-C₇)alkyl, or an O-protecting group; and
R² is —H, —(C₂-C₄)alkenyl, —(C₂-C₇)alkyl, —(C₁-C₇)alkyl-(C₃-C₇)cycloalkyl, —CN, —C(═O)O—(C₁-C₆)alkyl, —C(═O)O-phenyl or an N-protecting group.

In one embodiment, the disclosure is thus directed to a process for reducing the amount of a compound of formula (I) or a salt or a solvate thereof in a composition comprising compounds of formulae (I) and (II) or a salt or a solvate thereof as shown below:

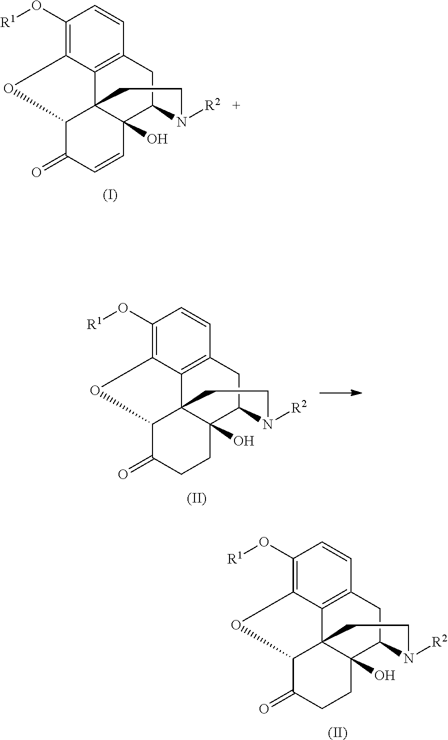

where:
R¹ is —H, (C₁-C₇)alkyl, or an O-protecting group; and
R² is —H, —(C₂-C₄)alkenyl, —(C₂-C₇)alkyl, —(C₁-C₇)alkyl-(C₃-C₇)cycloalkyl, —CN, —C(═O)O—(C₁-C₆)alkyl, —C(═O)O-phenyl or an N-protecting group.

In certain embodiments, the compound of formula (I) is a compound of formula (Ia):

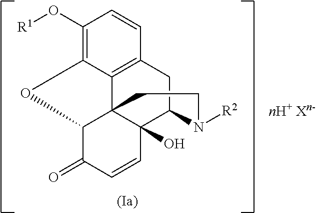

or a solvate thereof; where:
R¹ is —H, (C₁-C₇)alkyl, or an O-protecting group;
R² is —H, —(C₂-C₄)alkenyl, —(C₂-C₇)alkyl, —(C₁-C₇)alkyl-(C₃-C₇)cycloalkyl, —CN, —C(═O)O—(C₁-C₆)alkyl, —C(═O)O-phenyl or an N-protecting group;
Xⁿ⁻ is an anion selected from the group consisting of I⁻, F⁻, valerate, acetate, meconate, salicylate, barbiturate, Br⁻, succinate, tartrate, maleate, fumarate, citrate, NO₃⁻, Cl⁻, HSO₄⁻, SO₄²⁻, methanesulfonate, tosylate, trifluoroacetate, H₂PO₄⁻, HPO₄²⁻, PO₄³⁻, [(NH₄)HPO₄]⁻, [(NH₄)₂PO₄]⁻, oxalate, perchlorate, H₃CC(O)O⁻, HC(O)O⁻, and mixtures thereof; and
n is 1, 2 or 3.

In another embodiment, for the compound of formula (Ia):
Xⁿ⁻ is an anion selected from the group consisting of I⁻, F⁻, valerate, acetate, meconate, salicylate, barbiturate, Br⁻, succinate, tartrate, maleate, fumarate, citrate, NO₃⁻, Cl⁻, HSO₄⁻, SO₄²⁻, methanesulfonate, tosylate, trifluoroacetate, H₂PO₄⁻, HPO₄²⁻, [(NH₄)HPO₄]⁻, oxalate, perchlorate, H₃CC(O)O⁻, HC(O)O⁻, and mixtures thereof; and
n is 1 or 2.

In certain embodiments, the compound of formula (II) is a compound of formula (IIa):

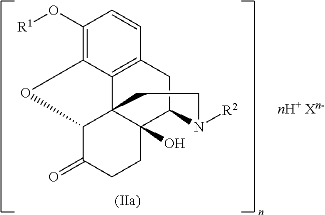

or a solvate thereof; where:
R¹ is —H, (C₁-C₇)alkyl, or an O-protecting group;
R² is —H, —(C₂-C₄)alkenyl, —(C₂-C₇)alkyl, —(C₁-C₇)alkyl-(C₃-C₇)cycloalkyl, —CN, —C(═O)O—(C₁-C₆)alkyl, —C(═O)O-phenyl or an N-protecting group;
Xⁿ⁻ is an anion selected from the group consisting of I⁻, F⁻, valerate, acetate, meconate, salicylate, barbiturate, Br⁻, succinate, tartrate, maleate, fumarate, citrate, NO₃⁻, Cl⁻, HSO₄⁻, SO₄⁻, methanesulfonate, tosylate, trifluoroacetate, H₂PO₄⁻, HPO₄²⁻, PO₄³⁻, [(NH₄)HPO₄]⁻, [(NH₄)₂PO₄]⁻, oxalate, perchlorate, H₃CC(O)O⁻, HC(O)O⁻, and mixtures thereof; and
n is 1, 2 or 3.

In another embodiment, for the compound of formula (IIa):
Xⁿ⁻ is an anion selected from the group consisting of I⁻, F⁻, valerate, acetate, meconate, salicylate, barbiturate, Br⁻, succinate, tartrate, maleate, fumarate, citrate, NO₃⁻, Cl⁻, HSO₄⁻, SO₄²⁻, methanesulfonate, tosylate, trifluoroacetate, H₂PO₄⁻, HPO₄²⁻, [(NH₄)HPO₄]⁻, oxalate, perchlorate, H₃CC(O)O⁻, HC(O)O⁻, and mixtures thereof; and
n is 1 or 2.

The salts of the compound of formula (Ia), the compound of formula (IIa), or the compound of formula (Ia) and the compound of formula (IIa) can be obtained by adding an acid H⁺ₙXⁿ⁻ to the reaction composition before hydrogenating step (b), during hydrogenating step (b), or before and during hydrogenating step (b).

The acid H⁺ₙXⁿ⁻ can be selected from the group consisting of H₂SO₄, H₃PO₄, HC(O)OH, and CH₃C(O)OH. In a preferred selection the acid H⁺ₙXⁿ⁻ is H₃PO₄.

The amount of acid can be from about 0.5 to about 10 molar equivalents based on the total molar equivalent of compounds of formulae (I) and (II) or (Ia) and (IIa), which are present in the composition. In one embodiment, the amount of acid is from about 1 to about 6 molar equivalents. In another embodiment, the amount of acid is from about 2 to about 3 molar equivalents. In another embodiment, the amount of acid is from about 2.2 to about 2.6 molar equivalents.

At least one of the compounds of formula (I), (Ia), (II) and (IIa) can be a hydrate of the compound of formula (I), (Ia), (II) and (IIa), respectively. In one embodiment, the hydrate is a monohydrate, dihydrate or trihydrate of at least one of the compounds of formula (I), (Ia), (II) and (IIa). In another embodiment, the hydrate is a dihydrate of at least one of the compounds of formula (I), (Ia), (II) and (IIa).

In one embodiment, the processes of the disclosure include a reaction composition, which optionally further comprises a solvent, which solvent can be selected from the group consisting of water, N-methylpyrrolidone (“NMP”), dimethylformamide (“DMF”), dimethylacetamide (“DMAc”), and mixtures thereof.

In certain embodiments, the process of the disclosure further comprises one or more of the following:
(a) an optional decolorizing step;
(c) an optional salt-breaking step; or
(a) an optional decolorizing step and (c) an optional salt-breaking step.

The process of the disclosure comprises a hydrogenating step (b), which may be performed in the presence of a hydrogenation reagent, which is preferably hydrogen.

Hydrogenating step (b) is typically performed in the presence of a hydrogenation catalyst, which is a transition metal-based catalyst. The transition metal-based catalyst can be selected from the group consisting of rhodium-, ruthenium-, platinum-, and palladium-based catalysts. The hydrogenation catalyst can be heterogeneous or homogenous. In one embodiment, the hydrogenation catalyst is heterogeneous. In one embodiment, the hydrogenation catalyst is on solid support. In one embodiment, the hydrogenation catalyst is selected as palladium on carbon. In another embodiment, the hydrogenation catalyst is selected as 5% palladium on carbon or 10% palladium on carbon.

If the activity of the hydrogenation catalyst needs to be adjusted, hydrogenating step (b) can be performed in the presence of a halide-containing compound (e.g., a chloride- or iodide-containing compound), which can be selected from the group consisting of ammonium chloride, ammonium iodide, sodium iodide, sodium chloride, sodium bromide, hydrochloric acid, potassium chloride, potassium iodide, barium chloride, lithium chloride, lithium iodide, calcium chloride and the like. In one embodiment, the halide-containing compound is selected from the group consisting of sodium iodide, sodium chloride, sodium bromide, and combinations thereof. In one embodiment, the halide-containing compound is sodium iodide.

In another embodiment, the halide-compound is a chloride-containing compound. In another embodiment, the halide-compound is an iodide-containing compound.

In another embodiment, hydrogenating step (b) is performed in the presence of an ammonium salt.

If a chloride-containing compound is present, in one embodiment the amount of chloride-containing compound is present at a level of from about 0.5 wt % to about 15.0 wt % based on the total weight of compounds of formulae (I) and (II). In other embodiments, the amount of chloride-containing compound is present at a level of from about 1.0 wt % to about 12.0 wt %, from about 2.5 wt % to about 10.0 wt %, from about 3.5 wt % to about 7.5 wt %, or from about 4.5 wt % to about 5.5 wt % based on the total weight of compounds of formulae (I) and (II). In one embodiment, the chloride-containing compound is sodium chloride.

If an iodide-containing compound is present, in one embodiment the amount of iodide-containing compound is present at a level of from about 0.0001 wt % to about 15 wt % based on the total weight of compounds of formulae (I) and (II). In other embodiments, the amount of iodide-containing compound is present at a level of from about 0.0005 wt % to about 5 wt %, from about 0.001 wt % to about 1 wt %, from about 0.002 wt % to about 0.5 wt %, or from about 0.0025 wt % to about 0.1 wt % based on the total weight of compounds of formulae (I) and (II). In one embodiment, the iodide-containing compounds is sodium iodide.

In a typical embodiment, the halide-containing compound is added before the addition of the hydrogenation catalyst.

After hydrogenating step (b), a filtration step is typically performed to remove the hydrogenation catalyst.

The process of the disclosure can optionally comprise an additional salt-breaking reaction step (c), where the pH-value of the composition is increased by addition of a base after the hydrogenation reaction. In one embodiment, the base is ammonium hydroxide.

In one embodiment, the pH-value is increased to from about 5.0 to about 10.0. In another embodiment, the pH is increased to from about 7.0 to about 9.5.

In certain embodiments, the base is added in at least one portion, preferably in two portions.

In one embodiment, a first portion of the base in salt-breaking step (c) is added to the product of hydrogenating step (b) until the pH is adjusted to from about 4.5 to about 5.5. In one embodiment, this increase in pH occurs when the product of hydrogenating step (b) is at a temperature of from about 20° C. to about 30° C.

In one embodiment, the second portion of the base in salt-breaking step (c) is added until the pH is adjusted of from about 7.0 to about 9.5, such as, e.g., a pH of from about 7.5 to about 9.0. In one embodiment, this increase in pH occurs while the temperature during addition of the second portion of the base is from about 40° C. to about 90° C. In one embodiment, the pH is raised to from about 7.5 to about 8.5 by addition of the second portion of the base while the temperature is from about 70° C. to about 80° C.

In certain embodiments, the product of the process is crystallized or precipitated during salt-breaking step (c), after salt-breaking step (c), or during and after salt-breaking step (c).

In certain embodiments, the product is isolated after salt-breaking step (c).

The isolated product can be optionally dried to reduce the water content of the composition of compounds of formulae (I) and (II) or the salts or solvates thereof. In one embodiment, the water content of product after drying is less than about 2 wt % based on the total weight of compounds of formulae (I) and (II) or the salts or solvates thereof.

The process of the disclosure can comprise a further optional decolorizing step (a), which comprises the addition of a decolorizing agent to the composition comprising compounds of formulae (I) and (II), or (Ia) and (IIa). Said decolorizing step (a) can be performed in at least one of before hydrogenating step (b), during hydrogenating step (b), or after hydrogenating step (b). In one embodiment, decolorizing step (a) is performed before hydrogenating step (b).

The decolorizing agent is selected from the group consisting of carbon-based decolorizing agents, aluminum-based decolorizing agents, and mixtures thereof. In one embodiment, the decolorizing agent is selected as an aluminum-based decolorizing agent. In another embodiment, the decolorizing agent is selected as a carbon-based decolorizing agent, preferably an activated granular carbon-based decolorizing agent. In another embodiment, the particle size of the activated granular carbon-based decolorizing agent is less than 75 μm.

In one embodiment, decolorizing step (a) is performed in the presence of a solvent which, in another embodiment, is selected from the group consisting of water, NMP, DMF, DMAc, and combinations thereof.

In certain embodiments, decolorizing step (a) is performed in the presence of an acid which, in another embodiment, is selected from the group consisting of H₂SO₄, H₃PO₄, HC(O)OH, and CH₃C(O)OH. In one embodiment, the acid is H₃PO₄.

The solvent and the acid of the hydrogenating step and the decolorizing step can be the same or different. In certain embodiments, the solvent and the acid of the hydrogenating step and the decolorizing step are identical. In certain embodiments, the amount of acid is from about 0.5 to about 10 molar equivalents based on the total molar equivalent of compounds of formulae (I) and (II) or (Ia) and (IIa). In other embodiments, the amount of acid is from about 1 to about 6 molar equivalents, about 2 to about 3 molar equivalents, or from about 2.2 to about 2.6 molar equivalents based on the total molar equivalent of compounds of formulae (I) and (II) or (Ia) and (IIa).

In certain embodiments, decolorizing step (a) is performed until the color of the solution of the reaction composition after decolorization is reduced from the color of the solution before decolorization, as indicated by a lower yellowness index (“YI”) after decolorization.

In one embodiment decolorizing step (a) leads to a reduction of the YI of the composition. In another embodiment, decolorizing step (a) is performed until the YI of the composition of compounds of formulae (I) and (II), or (Ia) and (IIa), or solvates thereof in the product is less than about 25, and preferably less than 10. The YI is typically measured at a concentration of about 4 mg/mL in a solvent, such as in from 0.01% to about 12% aqueous H₃PO₄ solution.

After decolorizing step (a), an optional filtration step, optionally combined with one or more washing steps, can be performed to remove the decolorizing agent.

In a preferred embodiment of the process of the disclosure, the compound of formula (I) is 14-hydroxynormorphinone or a salt or a solvate thereof, and the compound of formula (II) is noroxymorphone or a salt or a solvate thereof. The reaction composition comprising compounds of formulae (I) and (II) or salts or solvates thereof is dissolved in water, in the presence of from about 2.2 to about 2.6 molar equivalents of phosphoric acid (H₃PO₄).

The optional decolorizing step (a) can be performed before hydrogenating step (b), where the decolorizing agent is activated carbon, where preferably the total amount of decolorizing agent added is about 25 wt % based on the total weight of compounds of formulae (I) and (II). Decolorizing step (a) can be performed at about 90° C.

In one embodiment, hydrogenating step (b) is performed at a temperature of about 80° C. and the hydrogenation catalyst is 5 wt % palladium on carbon, where the amount of catalyst is about 1.8 wt % based on the total weight of compounds of formulae (I) and (II).

In another embodiment, hydrogenating step (b) is performed in the presence of a halide-containing compound, which is preferably a chloride-containing compound and even more preferably is sodium chloride. The halide-containing compound can be present in amount from about 4.5 wt % to about 5.5 wt % based on the total weight of compounds of formulae (I) and (II). The addition of the halide-containing compound is preferred if a ring-opening by-product of formula (IV) would otherwise be observed being formed during hydrogenation.

In this embodiment, the addition of base in the salt-breaking step (c) is performed after hydrogenating step (b). Preferably, the base is added in two portions. A first portion of base, which is preferably ammonium hydroxide, is added at a temperature of about 25° C., until a pH of about 5.0 is reached. Subsequently, a second portion of the base is added at a temperature of about 75° C., until a pH of about 8.0 is reached.

In a preferred embodiment, the compound of formula (I) is 14-hydroxynormorphinone or a salt or a solvate thereof, the compound of formula (II) is noroxymorphone or a salt or a solvate thereof, and the isolated product after the salt-breaking step (c) is anhydrous or dihydrate noroxymorphone, and preferably is anhydrous noroxymorphone.

The process of the disclosure, which includes a composition comprising compounds of formulae (I) and (II) or salts or solvates thereof, can further comprise controlled or reduced amounts of compounds of formula (III):

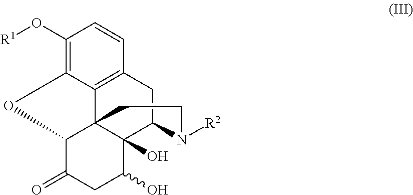

or a salt or a solvate thereof; where:
R¹ is —H, (C₁-C₇)alkyl, or an O-protecting group; and
R² is —H, —(C₂-C₄)alkenyl, —(C₂-C₇)alkyl, —(C₁-C₇)alkyl-(C₃-C₇)cycloalkyl, —CN, —C(═O)O—(C₁-C₆)alkyl, —C(═O)O-phenyl or an N-protecting group.

The process of the disclosure, which includes a composition comprising compounds of formulae (I) and (II) or salts or solvates thereof can further comprise controlled or reduced amounts of compounds of formula (IV):

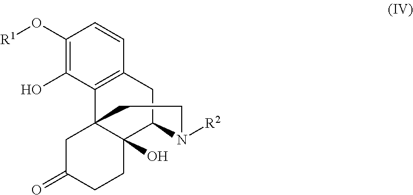

or a salt or a solvate thereof; where:
R¹ is —H, (C₁-C₇)alkyl, or an O-protecting group; and
R² is —H, —(C₂-C₄)alkenyl, —(C₂-C₇)alkyl, —(C₁-C₇)alkyl-(C₃-C₇)cycloalkyl, —CN, —C(═O)O—(C₁-C₆)alkyl, —C(═O)O-phenyl or an N-protecting group.

In one embodiment, the compound of formula (IV) is a compound of formula (IVa):

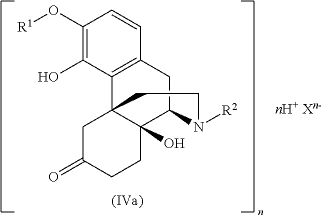

or a solvate thereof; where:
R¹ is —H, (C₁-C₇)alkyl, or an O-protecting group;
R² is —H, —(C₂-C₄)alkenyl, —(C₂-C₇)alkyl, —(C₁-C₇)alkyl-(C₃-C₇)cycloalkyl, —CN, —C(═O)O—(C₁-C₆)alkyl, —C(═O)O-phenyl or an N-protecting group;
Xⁿ⁻ is an anion selected from the group consisting of I⁻, F⁻, valerate, acetate, meconate, salicylate, barbiturate, Br⁻, succinate, tartrate, maleate, fumarate, citrate, NO₃⁻, Cl⁻, HSO₄⁻, SO₄⁻, methanesulfonate, tosylate, trifluoroacetate, H₂PO₄⁻, HPO₄²⁻, PO₄³⁻, [(NH₄)HPO₄]⁻, [(NH₄)₂PO₄]⁻, oxalate, perchlorate, H₃CC(O)O⁻, HC(O)O⁻, and mixtures thereof; and
n is 1, 2 or 3.

In another embodiment, for the compound of formula (IVa):
Xⁿ⁻ is an anion selected from the group consisting of I⁻, F⁻, valerate, acetate, meconate, salicylate, barbiturate, Br⁻, succinate, tartrate, maleate, fumarate, citrate, NO₃⁻, Cl⁻, HSO₄⁻, SO₄²⁻, methanesulfonate, tosylate, trifluoroacetate, H₂PO₄⁻, HPO₄²⁻, [(NH₄)HPO₄]⁻, oxalate, perchlorate, H₃CC(O)O⁻, HC(O)O⁻, and mixtures thereof; and
n is 1 or 2.

In a preferred embodiment, the compound of formula (IV) is 3,4,14-trihydroxymorphinan-6-one.

The processes of the disclosure lead to reduced amounts of a compound of formula (I) or a salt or a solvate thereof in the reaction composition comprising compounds of formulae (I) and (II) or salts or solvates thereof. The amount of compounds of formula (I) or a salt or a solvate thereof in the starting material is typically more than 500 ppm, more than 1,000 ppm, or more that 10,000 ppm. In certain embodiments, the amount of a compound of formula (I) or a salt or a solvate thereof in the product is less than about 150 ppm, less than about 100 ppm, less than about 75 ppm, less than about 50 ppm, less than about 40 ppm, less than about 35 ppm, less than about 25 ppm, less than about 10 ppm, or less than 5 ppm.

In other embodiments, the processes of the disclosure are performed until the amount of a compound of formula (I) or a salt or a solvate thereof in the product is less than the specified limit set by a regulatory authority such as, for example, less than about 90 ppm, or less than about 75 ppm, less than about 40 ppm, or less than about 35 ppm.

In other embodiments, the processes of the disclosure are performed until the amount of 14-hydroxynormorphinone, or a salt or a solvate thereof in the product is less than about 90 ppm, less than about 75 ppm, less than about 50 ppm, less than about 40 ppm, less than about 35 ppm or less than about 10 ppm, or less than 5 ppm. In other embodiments, the processes of the disclosure are performed until the amount of 14-hydroxynormorphinone, or a salt or a solvate thereof in the product is less than about 100 ppm, less than 75 ppm, less than about 50 ppm, or less than about 25 ppm.

In one embodiment of the disclosure, the compound of formula (I) is 14-hydroxynormorphinone or a salt or a solvate thereof and the compound of formula (II) is noroxymorphone or a salt or a solvate thereof. The reaction product containing less than about 100 ppm, less than about 75 ppm, less than about 50 ppm, or less than about 25 ppm 14-hydroxynormorphinone, or a salt or a solvate thereof, is further converted to a product of naloxone or a salt or a solvate thereof containing less than about 100 ppm, less than about 75 ppm, less than about 50 ppm, or less than about 25 ppm 7,8-didehydronaloxone or a salt or a solvate thereof.

In another embodiment, the compound of formula (I) is 14-hydroxynormorphinone or a salt or a solvate thereof and the compound of formula (II) is noroxymorphone. The reaction product containing less than about 100 ppm, less than about 75 ppm, less than about 50 ppm, or less than about 25 ppm 14-hydroxynormorphinone or a salt or a solvate thereof is further converted to a product of naltrexone or a salt or a solvate thereof containing less than about 100 ppm, less than about 75 ppm, less than about 50 ppm, or less than about 25 ppm 7,8-didehydronaltrexone or a salt or a solvate thereof.

In yet other embodiments, the disclosure is directed to a composition comprising compounds of formulae (I) and (II) or salts or solvates thereof (e.g., a 14-hydroxynormophinone or noroxymorphone salt, respectively), where the amount of a compound of formula (I) or a salt or a solvate thereof relative to the amount of compounds of formula (II) in the composition is less than about 200 ppm, less than about 150 ppm, less than about 100 ppm, less than about 75 ppm, less than about 50 ppm, less than about 40 ppm, less than about 35 ppm, less than about 25 ppm, less than about 10 ppm, or less than 5 ppm, and where the composition optionally comprises a compound of formula (III) (e.g., 8-hydroxynoroxymorphone), a compound of formula (IV), a compound of formula (III) and of formula (IV), or a salt or a solvate thereof.

In yet another embodiment, the disclosure is directed to a composition of the disclosure comprising compounds of formulae (I) and (II) or salts or solvates thereof (e.g., an 14-hydroxynormorphinone or noroxymorphone salt, respectively), obtainable by a process of the disclosure.

In a further embodiment, compositions comprising compounds of formulae (I) and (II) or salts or solvates thereof are disclosed, which can be used as intermediate or starting materials in the preparation of a morphinan derivative or pharmaceutically acceptable salts or solvates thereof, and/or for preparing a medicament containing the composition comprising compounds of formulae (I) and (II) or the pharmaceutically acceptable salts or solvates thereof, or containing another morphinan derivative or a pharmaceutically acceptable salt or solvate thereof, such as naloxone or naltrexone or salts or solvates thereof.

In a further embodiment, the compositions comprising compounds of formulae (I) and (II) or pharmaceutically acceptable salts or solvates thereof in accordance with the disclosure are used as medicaments.

In one embodiment of the disclosure, a composition comprising compounds of formulae (I) and (II) or pharmaceutically acceptable salts thereof is useful as a medicament in the treatment or prevention of pain; addiction; cough; constipation; diarrhea; insomnia associated with or caused by pain, cough or addiction; depression associated with or resulting from pain, cough or addiction; or a combination of two or more of the foregoing. In another embodiment of the disclosure, a composition comprising compounds of formulae (I) and (II) or pharmaceutically acceptable salts thereof is useful as a medicament in the treatment of pain; addiction; cough; constipation; diarrhea; insomnia associated with or caused by pain, cough or addiction; depression associated with or resulting from pain, cough or addiction; or a combination of two or more of the foregoing. In another embodiment of the disclosure, a composition comprising compounds of formulae (I) and (II) or pharmaceutically acceptable salts thereof is useful as a medicament in the prevention of pain; addiction; cough; constipation; diarrhea; insomnia associated with or caused by pain, cough or addiction; depression associated with or resulting from pain, cough or addiction; or a combination of two or more of the foregoing.

In another embodiment, said composition is useful as a medicament in the treatment or prevention of pain. In another embodiment, said composition is useful as a medicament in the treatment of pain. In another embodiment, said composition is useful as a medicament in the prevention of pain.

A further embodiment provides a method for treating or preventing a medical condition in an animal comprising administering to an animal in need thereof an effective amount of a composition comprising compounds of formulae (I) and (II) or pharmaceutically acceptable salts or solvates thereof, where the condition is selected from the group consisting of pain; addiction; cough; constipation; diarrhea; insomnia associated with, caused by, or associated with and caused by pain, cough or addiction; depression associated with or resulting from pain, cough or addiction; and combinations of two or more of the foregoing. Another embodiment provides a method for treating a medical condition in an animal comprising administering to an animal in need thereof an effective amount of a composition comprising compounds of formulae (I) and (II) or pharmaceutically acceptable salts or solvates thereof, where the condition is selected from the group consisting of pain; addiction; cough; constipation; diarrhea; insomnia associated with, caused by, or associated with and caused by pain, cough or addiction; depression associated with or resulting from pain, cough or addiction; and combinations of two or more of the foregoing. Another embodiment provides a method for preventing a medical condition in an animal comprising administering to an animal in need thereof an effective amount of a composition comprising compounds of formulae (I) and (II) or pharmaceutically acceptable salts or solvates thereof, where the condition is selected from the group consisting of pain; addiction; cough; constipation; diarrhea; insomnia associated with, caused by, or associated with and caused by pain, cough or addiction; depression associated with or resulting from pain, cough or addiction; and combinations of two or more of the foregoing.

In another embodiment, said method is useful for treating or preventing pain in an animal and comprises administering to an animal in need thereof an effective amount of a composition comprising compounds of formulae (I) and (II) or pharmaceutically acceptable salts or solvates thereof. In another embodiment, said method is useful for treating pain in an animal and comprises administering to an animal in need thereof an effective amount of a composition comprising compounds of formulae (I) and (II) or pharmaceutically acceptable salts or solvates thereof. In another embodiment, said method is useful for preventing pain in an animal and comprises administering to an animal in need thereof an effective amount of a composition comprising compounds of formulae (I) and (II) or pharmaceutically acceptable salts or solvates thereof.

### BRIEF DESCRIPTION OF THE DRAWINGS

FIG. 1A: HPLC chromatogram obtained according to the HPLC method of Example 1.1. for the decolorized sample of noroxymorphone from Example 5.4. The peak at 11.663 minutes denotes the presence of 14-hydroxynormorphinone (Impurity 1). The peak at 8.360 minutes denotes the presence of 8-hydroxynoroxymorphone (Impurity 2).

FIG. 1B: Enlargement of the 1-15 minute portion of the HPLC chromatogram shown in FIG. 1A.

FIG. 2A: HPLC chromatogram obtained according to the HPLC method of Example 1.1. for the naloxone end-product of hydrogenation from Example 5.4. A peak at about 11.70 minutes, corresponding to Impurity 1, is absent. The peak at 8.214 minutes denotes the presence of 8-hydroxynoroxymorphone (Impurity 2).

FIG. 2B: Enlargement of the 1-15 minute portion of the HPLC chromatogram shown in FIG. 2A.

### DETAILED DESCRIPTION

### Definitions

Unless otherwise specified, the following abbreviations and definitions are used in the context of the disclosure.

A “morphinan derivative” in its broadest sense encompasses all compounds usually designated with said term in the art, including morphinan derivatives which act as an agonist on opioid receptors and morphinan derivatives which act as an antagonist on opioid receptors. Preferably, the morphinan derivatives are 4,5-epoxymorphinan derivatives. The term “morphinan derivative” also includes single compounds and compositions of compounds selected from the group of morphinan derivatives, and combinations of any of the foregoing, such as a combination of mixed opioid agonists-antagonists, or a combination of mixed partial opioid agonists-antagonists, and single compounds having mixed pharmacologies. The term “morphinan derivative” also encompasses any stereoisomers and salts thereof, and mixtures of any of the foregoing. Morphinan derivative opioid agonists include, e.g., oxymorphone, noroxymorphone, hydromorphone, nalfurafine and salts of any of the foregoing. In certain embodiments, the morphinan derivative is noroxymorphone or a salt thereof, such as, e.g., noroxymorphone hydrogen phosphate. Morphinan derivative opioid antagonists include, e.g., naltrexone, methylnaltrexone, naloxone, nalmefene, and salts of any of the foregoing. The term “morphinan derivative” shall preferably encompass a compound having the following scaffold (which is designated as a “morphine scaffold”):

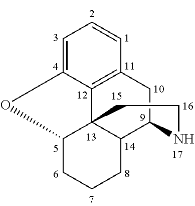 [SMILES: c1cc2c3c(c1)O[C@H]1CCCC4[C@@H](C2)NCC[C@@]341]

The degree of unsaturation in the ring formed by atoms 5, 6, 7, 8, 14 and 13 may vary.

Thus, the term “morphinan derivative” in its broadest sense encompasses compounds of formulae (I), (Ia), (II), (IIa), (III), (IIIa), (IV), (IVa), (V) and (VI) as well as compositions containing one or more of the compounds of formulae (I), (Ia), (II), (IIa), (III), (IIIa), (IV), (IVa), (V) and (VI), including the salts thereof. In the processes of the disclosure, morphinan derivatives can serve as starting materials, intermediates, or final products. They can (for example, if compositions comprising compounds of formulae (I) and (II)) serve as intermediates or final products in the process of the disclosure and as a starting material in another process. Whenever a “process for reducing the amount of a morphinan derivative” is mentioned herein, it will be clear from the context which morphinan derivative is reduced in amount. In a narrower sense, the term “morphinan derivative” shall designate compounds of formulae (I) and (II) and the pharmaceutically acceptable salts and solvates thereof. One of the objects of the disclosure is the use of said morphinan derivative compositions comprising one or more of the compounds of formulae (I), (Ia), (II), (IIa), (III), (IIIa), (IV), (IVa), (V) and (VI) and the salts and solvates thereof, as starting materials or intermediate materials in the preparation of morphinan derivatives. Another object of the disclosure is to provide pharmaceutically acceptable salts or solvents of compositions comprising compounds of formulae (I) and (II), which can serve as APIs (e.g., naloxone or a pharmaceutically acceptable salt thereof), and their immediate precursors (e.g., compositions comprising compounds of formulae (I) and (II) containing 14-hydroxymorphinone). Hence, the term “morphinan derivative” will also be used to refer to compounds of formula (II) and the salts and solvates thereof.

As used herein, reference to a compound of formulae (I), (Ia), (II), (IIa), (III), (IIIa), (IV), (IVa), (V), (VI), and the like, unless otherwise indicated, also includes the respective salts thereof. Also, as used herein, reference to a named compound (e.g., naloxone, naltrexone, etc.), unless otherwise indicated, also includes the salts thereof.

The “threshold amount” of compositions comprising compounds of formulae (I) and (II) in pharmaceutical compositions and dosage forms is set by regulatory authorities such as the U.S. Food and Drug Administration (FDA) and the European Medicines Agency (EMA), and can be learned from the latest version of the FDA or EMA monographs (“Monographs”) or, if certain compositions comprising compounds of formulae (I) and (II) are not addressed in said Monographs, then from the latest version of the ICH Guidelines. For example, for a naloxone hydrochloride API, the current threshold amount according to the EMA is 75 ppm of the ABUK 7,8-di-dehydronaloxone. In contrast, for the monitored ABUKs in the US, the threshold amount of the ABUK 14-hydroxymorphinone depends upon the product that is being regulated and refers to the amount above which the FDA will not approve the product for use and sale to the public.

The term “8-hydroxy compound” means a compound containing a hydroxyl group in position 8 of the 4,5-epoxymorphinan scaffold. In a narrower sense, it means a compound having the structure of formula (III):

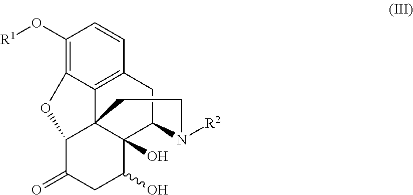

or a salt or a solvate thereof;
where:
R¹ is H or an O-protecting group selected from the group consisting of acetate, ethyloxycarbonyl, pivolate, benzoate, tert-butyldiphenylsilyl (“TBDPS”), trimethylsilyl (“TMS”), triethylsilyl (“TES”), tert-butyldimethylsilyl (“TBS”), benzyl (“Bn”), triphenylmethyl (“Tr”) and tert-butyl; and
R² is —H, (C₂-C₄)alkenyl, (C₂-C₇)alkyl, —(C₁-C₇)alkyl-(C₃-C₇)cycloalkyl, —CN, —C(═O)O—(C₁-C₆)alkyl, —C(═O)O-phenyl or a N-protecting group, which is selected from the group consisting of acetamide, ethyloxycarbonyl, tert-butyloxycarbonyl (“Boc”), carbobenzyloxy (“Cbz”), 9-fluorenylmethyloxycarbonyl (“Fmoc”), allyloxycarbonyl (“Alloc”), tosyl, benzenesulfonyl, trifluoromethylcarbonyl, and 2,2,2-trichloroethoxycarbonyl (“TroC”).

The term “8-hydroxy compound” includes the 8α-hydroxy compound of formula (V), the 8β-hydroxy compound of formula (VI), or a combination of the 8-hydroxy compounds of formula (V) and formula (VI):

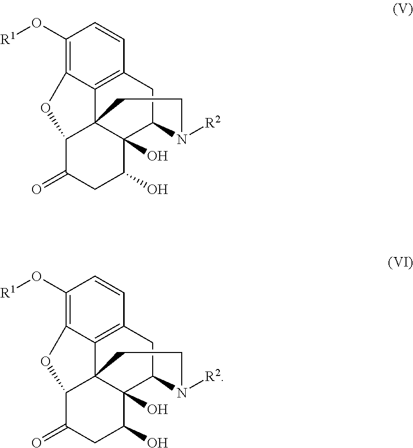

The term “8-hydroxy compound” includes not only the compounds of formula (IIIa) but also the salts of formula (IIIa) or solvates thereof:

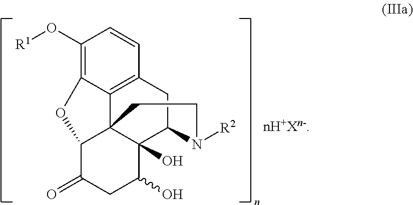

“—(C₁-C₇)alkyl” means a straight chain or branched non-cyclic hydrocarbon having 1, 2, 3, 4, 5, 6, or 7 carbon atoms. Typical straight chain —(C₁-C₇)alkyl groups include -methyl, -ethyl, -n-propyl, -n-butyl, -n-pentyl, -n-hexyl, and -n-heptyl. A branched alkyl means that one or more straight chain —(C₁-C₅)alkyl groups, such as methyl, ethyl or propyl, replace one or both of the hydrogens in one or more —CH₂— groups of a straight chain alkyl. The total number of carbon atoms in a branched chain alkyl is 3, 4, 5, 6, or 7 carbon atoms. Typical branched —(C₁-C₇)alkyl groups include -iso-propyl, -sec-butyl, -iso-butyl, -tert-butyl, -iso-pentyl, -neopentyl, 1-methylbutyl, 2-methylbutyl, 3-methylbutyl, 1,1-dimethylpropyl, 1,2-dimethylpropyl, 1-methylpentyl, 2-methylpentyl, 3-methylpentyl, 4-methylpentyl, 1-ethylbutyl, 2-ethylbutyl, 3-ethylbutyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-methylhexyl, 2-methylhexyl, 3-methylhexyl, 4-methylhexyl, 5-methylhexyl, 1,2-dimethylpentyl, and 1,3-dimethylpentyl.

“—(C₂-C₇)alkyl” means a straight chain or branched non-cyclic hydrocarbon having 2, 3, 4, 5, 6, or 7 carbon atoms. Typical straight chain —(C₂-C₇)alkyl groups include -ethyl, -n-propyl, -n-butyl, -n-pentyl, -n-hexyl, and -n-heptyl. A branched alkyl means that one or more straight chain —(C₁-C₅)alkyl groups, such as methyl, ethyl or propyl, replace one or both hydrogens in one or more —CH₂— groups of a straight chain alkyl. The total number of carbon atoms in a branched chain alkyl is 3, 4, 5, 6, or 7 carbon atoms. Typical branched —(C₂-C₇)alkyl groups include -iso-propyl, -sec-butyl, -iso-butyl, -tert-butyl, -iso-pentyl, -neopentyl, 1-methylbutyl, 2-methylbutyl, 3-methylbutyl, 1,1-dimethylpropyl, 1,2-dimethylpropyl, 1-methylpentyl, 2-methylpentyl, 3-methylpentyl, 4-methylpentyl, 1-ethylbutyl, 2-ethylbutyl, 3-ethylbutyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-methylhexyl, 2-methylhexyl, 3-methylhexyl, 4-methylhexyl, 5-methylhexyl, 1,2-dimethylpentyl, and 1,3-dimethylpentyl.

“—(C₁-C₆)alkyl” means a straight chain or branched non-cyclic hydrocarbon having 1, 2, 3, 4, 5 or 6 carbon atoms. Typical straight chain —(C₁-C₆)alkyl groups include -methyl, -ethyl, -n-propyl, -n-butyl, -n-pentyl and -n-hexyl. Typical branched —(C₁-C₆)alkyl groups include -iso-propyl, -sec-butyl, -iso-butyl, -tert-butyl, -iso-pentyl, -iso-hexyl, -neopentyl, 1-methylbutyl, 2-methylbutyl, 3-methylbutyl, 1,1-dimethylpropyl, 1,2-dimethylpropyl, 1-methylpentyl, 2-methylpentyl, 3-methylpentyl, 4-methylpentyl, 1-ethylbutyl, 2-ethylbutyl, 3-ethylbutyl, 1,1-dimethylbutyl, 1,2-dimethylbutyl, 1,3-dimethylbutyl, 2,2-dimethylbutyl, 2,3-dimethylbutyl, and 3,3-dimethylbutyl.

“—(C₁-C₅)alkyl” means a straight chain or branched non-cyclic hydrocarbon having 1, 2, 3, 4, or 5 carbon atoms. Typical straight chain —(C₁-C₅)alkyl groups include -methyl, -ethyl, -n-propyl, -n-butyl and -n-pentyl. Typical branched —(C₁-C₅)alkyl groups include -iso-propyl, -sec-butyl, -iso-butyl, -tert-butyl, -iso-pentyl, -neopentyl, 1-methylbutyl, 2-methylbutyl, 3-methylbutyl, 1,1-dimethylpropyl, and 1,2-dimethylpropyl.

“—(C₁-C₄)alkyl” means a straight chain or branched non-cyclic hydrocarbon having 1, 2, 3, or 4 carbon atoms. Typical straight chain —(C₁-C₄)alkyl groups include -methyl, -ethyl, -n-propyl, and -n-butyl. Typical branched —(C₁-C₄)alkyl groups include -iso-propyl, -sec-butyl, -iso-butyl, and -tert-butyl.

“—(C₂-C₄)alkenyl” means a straight chain or branched non-cyclic hydrocarbon having 2, 3, or 4 carbon atoms and including at least one carbon-carbon double bond. Representative straight chain and branched —(C₂-C₄)alkenyls include -vinyl, -allyl, -1-butenyl, -2-butenyl, -3-butenyl, -iso-butylenyl, and -1,3 -butadienyl.

“—(C₃-C₇)cycloalkyl” means a saturated or partially unsaturated (containing, e.g., one, two or three double bonds) cyclic hydrocarbon containing 1, 2, or 3 rings each ring having 3, 4, 5, 6, or 7 carbon atoms, respectively. Typical —(C₃-C₇)cycloalkyl groups include -cyclopropyl, -cyclobutyl, -cyclopentyl, -cyclohexyl, -cycloheptyl, -norbornyl, -cyclopropenyl, -cyclobutenyl, -cyclopentenyl, -cyclopentadienyl, -cyclohexenyl, -cyclohexadienyl, -cycloheptadienyl, and -cycloheptatrienyl.

“N-protecting groups” or “nitrogen protecting groups” include any group which may be suitable to protect a nitrogen from taking part in a reaction and which can be removed during or after the reaction. Examples of such protecting groups include acetamide, ethyloxycarbonyl, Boc, Cbz, Fmoc, Alloc, tosyl, benzenesulfonyl, trifluoromethylcarbonyl, TroC, and the like. Further examples can be found in Wuts and Greene, Green's Protective Groups in Organic Synthesis, Wiley-Interscience, Hoboken, N.J., 4^{th} Edition, Chapters 7, 10 (2007).

“O-protecting groups” or “oxygen protecting groups” include any group which may be suitable to protect an oxygen from taking part in a reaction, and which can be removed during or after the reaction. Examples of such protecting groups include acetate, ethyloxycarbonyl, pivolate, benzoate, TBDPS, TMS, TES, TBS, Bn, triphenylmethyl, tert-butyl and the like. Further examples can be found in Wuts and Greene, Greene's Protective Groups in Organic Synthesis, Wiley-Interscience, Hoboken, N.J., 4^{th} Edition, Chapters 2-5, 10 (2007).

The term “halide-containing compound” means a binary compound, of which one part is a halogen atom, i.e., fluorine, chlorine, bromine, or iodine, and the other part is an element or radical that is less electronegative (or more electropositive) than the halogen, to make a fluoride, chloride, bromide, or iodide compound. The term “halide-containing compound” encompasses halogen salts, which can include, but are not limited to ammonium chloride, sodium iodide, sodium chloride, sodium bromide, potassium iodide, potassium chloride, lithium iodide, lithium chloride, and hydrochloric acid.

The term “solvate” means at least one of a combination, a physical association, or a solvation product of a compound or a salt of the disclosure with a solvent molecule. Solvates can be formed with a single solvent or multiple solvents. The physical association involves varying degrees of ionic and covalent bonding, including hydrogen bonding. In certain instances, the solvate can be isolated, such as when one or more solvent molecules are associated with the compound or a salt of the disclosure in a precipitate or are incorporated into the crystal lattice of a crystalline solid. Thus, the term “solvate” encompasses both non-isolated and isolatable solvates. The molar ratio of solvent molecule(s) per compound molecule can vary. The molar ratio of solvent to compound or a salt thereof in the solvate can be 1 (e.g., in a monohydrate), more than 1 (e.g., 2, 3, 4, 5 or 6 in a polyhydrate), or less than 1 (e.g., 0.5 in a hemihydrate). The molar ratio need not be an integer ratio, it can also be, e.g., 0.5 (as in a hemihydrate) or 2.5 (as in a penta-hemihydrate). For example, 2 water molecules per molecule of noroxymorphone-dihydrogen phosphate are associated with noroxymorphone-dihydrogen phosphate dihydrate. Compounds of any one of formulae (I)-(VI) may be present as solvated forms with a pharmaceutically acceptable solvent, such as water, methanol, ethanol, and the like. It is intended that the disclosure includes both solvated and unsolvated forms of compounds of any one of formulae (I)-(VI). Solvates typically can function as pharmacological equivalents.

In certain embodiments, the solvate of compounds of any one of formulae (I)-(VI) of the disclosure is a “hydrate”. A “hydrate” relates to a particular subgroup of solvates where the solvent molecule is water. For example, a hydrate includes a hemihydrate, monohydrate, dihydrate, trihydrate, tetrahydrate, pentahydrate or hexahydrate, or a hydrate where the ratio of water per molecule is not necessarily an integer, but is from 0.5 to 10.0. In certain embodiments, the solvate is a hydrate where the ratio of water per molecule is from 1 to 8. In certain embodiments, the solvate is a hydrate where the ratio of water per compound or salt molecule is from 1 to 5. In certain embodiments, the solvate is a hydrate where the ratio of water per molecule is from 1 to 3, e.g., a mono-, di-, or trihydrate. In certain embodiments, it is a monohydrate. In certain embodiments, it is a dihydrate. Preparation of solvates is known in the art. See, for example, Caira et al., J. Pharmaceut. Sci., 93(3): 601-611 (2004), which describes the preparation of solvates of fluconazole with ethyl acetate and with water. Similar preparations of solvates, hemisolvates, hydrates, and the like are described by van Tonder et al., AAPS Pharm. Sci. Tech., 5(1): Article 12 (2004), and Bingham et al., Chem. Commun.: 603-604 (2001). Analytical techniques such as infrared spectroscopy can be used to confirm the presence of the solvent in a crystal or solvate.

The terms “crystallizing”, “crystallize”, and “crystallization” refer to a process of forming solid crystals precipitating from a solution, where crystal(s) mean a solid material, where the constituent compounds, salts thereof, solvates thereof or any combination thereof are arranged in a regular pattern, which extends in all three spatial dimensions.

The terms “precipitating”, “precipitate”, and “precipitation” shall encompass “crystallizing”, “crystallize”, and “crystallization” unless stated otherwise. In certain embodiments, the precipitate described herein is amorphous. In certain embodiments, the precipitate is a mixture of amorphous and crystalline components. In certain embodiments, the precipitate described herein is crystalline.

For purposes of the disclosure, the term “ppm” means parts per million, 1 ppm corresponding to 10⁻⁶. For purposes of the present application, unless otherwise indicated, the numeric ppm value is a ppm weight value and the numeric ppm weight value of a morphinan derivative contained in a composition containing more than one morphinan derivative is given relative to the total weight of all of the morphinan derivatives, where the basis for calculation is the free base form. For compositions where the morphinan derivative is a salt or solvate, the ppm value refers to the numeric ppm based on the weight of the morphinan derivative free base portion in the salt or solvate relative to the total weight of the free base portion(s) of morphinan derivatives in the composition. For example, 20 ppm didehydronaloxone hydrochloride in a composition containing naloxone hydrochloride dihydrate and didehydronaloxone hydrochloride refers to 20 ppm didehydronaloxone free base relative to the total amount of naloxone free base and didehydronaloxone free base.

For purposes of the disclosure, a high performance liquid chromatography (“HPLC”) method can be performed to determine low ppm values. For example, a quantitation of 14-hydroxynormorphinone in a morphinan derivative composition can be achieved by comparison against an external standard with a known purity as described in Example 1.1.

Preferably, the HPLC method for determination of an ABUK or ABUKs has a limit of detection (“LOD”) of 10 ppm and preferably of 5 ppm ABUK. In another preferred embodiment, the limit of quantitation (“LOQ”) of the HPLC method is no more than 20 ppm, and preferably no more than 10 ppm. In a particularly preferred embodiment, the HPLC method has a LOD of 5 ppm and a LOQ of 10 ppm. Consequently, in this particularly preferred embodiment, the ABUK can be quantified down to 10 ppm. If a peak is detectable, but below the LOQ of 10 ppm, then the peak can only be described as “<10 ppm”. If the peak is below the detection limit of 5 ppm, then the peak is categorized as non detectable (“ND”).

In preferred embodiments, the HPLC method described in Example 1.3. is used for determination of ppm values for 7,8-didehydronaloxone in a naloxone composition.

In preferred embodiments, the HPLC method described in Example 1.4. is used for determination of ppm values for 7,8-didehydronaltrexone in a naltrexone composition.

The term “API” means “active pharmaceutical ingredient” (e.g., naloxone hydrochloride) and shall be used in its broadest sense as a synonym for a pharmaceutically active compound. When an API is used in preparing a pharmaceutical composition or dosage form, the API is the pharmaceutically active component of said pharmaceutical composition or dosage form. Pharmaceutical compositions or dosage forms containing an API may be approved by a governmental agency for sale and use in a patient (e.g., a mammal such as a human). Examples of APIs described herein include, e.g., pharmaceutically acceptable salts and solvates thereof, e.g., naloxone, naloxone hydrochloride, naltrexone or naltrexone hydrochloride.

The term “pharmaceutical composition” means a composition which contains an API and is suitable for use in a patient (e.g., a mammal such as a human). It may be approved by a governmental agency for sale and use in a patient. Examples for pharmaceutical compositions described herein are naloxone, naloxone hydrochloride, naltrexone or naltrexone hydrochloride. Pharmaceutical compositions can be compositions prepared according to the disclosure if they comply with regulatory requirements for pharmaceutical compositions containing the same API. In addition to an API, pharmaceutical compositions typically include one or more pharmaceutically acceptable carriers or excipients, as known in the art.

The term “salt” means a compound comprising at least one cation (e.g., one or two 14-hydroxynormorphinone cations resulting from protonation of 14-hydroxynormorphinone (free base) by a Bronsted acid (e.g., H₃PO₄)) and at least one anion (e.g., a hydrogen phosphate or dihydrogen phosphate anion). A salt can be the result of the neutralization reaction between an acid and a base (e.g., a Bronsted acid and a Bronsted base, or a Lewis acid and a Lewis base). In its solid form, the salt can form a precipitate or can have a crystalline structure. The term “salt” encompasses all salts of the disclosed compound. In one embodiment, salts of the disclosure include all pharmaceutically acceptable salts of the disclosed compounds, particularly when referring to a salt of a compound which can serve as an API. Examples of salts include inorganic and organic acid addition salts and basic salts. The pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; inorganic acid salts such as bromide, chloride, iodide, fluoride, nitrate, hydrochloride, hydrobromide, phosphate, hydrogen phosphate, dihydrogen phosphate, diammonium phosphate, ammoniumhydrogen phosphate, oxalate, perchlorate, sulfate, hydrogen sulfate and the like; organic acid salts such as citrate, lactate, succinate, tosylate, tartrate, maleate, fumarate, mandelate, acetate, dichloroacetate, trifluoroacetate, oxalate, formate and the like; sulfonates such as methanesulfonate, benzenesulfonate, p-toluensulfonate and the like; and amino acid salts such as arginate, asparginate, glutamate and the like.

Acid addition salts can be formed by mixing a solution of the particular compound of the disclosure with a solution of a pharmaceutically acceptable non-toxic acid such as formic acid, sulfuric acid, hydrochloric acid, fumaric acid, maleic acid, succinic acid, acetic acid, citric acid, tartaric acid, carbonic acid, phosphoric acid, oxalic acid, dichloroacetic acid, and the like. Basic salts can be formed by mixing a solution of the particular compound of the disclosure and a non-toxic base such as sodium hydroxide, potassium hydroxide, choline hydroxide, sodium carbonate and the like. The term “salt” includes anhydrous, solvated, or hydrated forms of the salt.

Whenever a solution or mixture containing a salt is mentioned, the term “salt” shall also encompass the dissolved form of the salt. Whenever a 14-hydroxynormorphinone salt is mentioned, this refers to a salt containing a 14-hydroxynormorphinone cation, resulting, e.g., from protonation of the 14-hydroxynormorphinone free base. The same applies to other salts containing a cation with a morphine scaffold. One example of a salt of the disclosure is a compound of formula (IIa) (which corresponds to a salt of compounds of formula (II)) or a solvate thereof. An example of such compound of formula (IIa) is a salt, e.g., noroxymorphone hydrogen phosphate, which consists of two molecules of noroxymorphone and one molecule of hydrogen phosphate. In this salt, the cation results from the protonation of two molecules of noroxymorphone and the anion is the resulting hydrogen phosphate. In certain embodiments of the disclosure, a salt which is a composition comprising compounds of formulae (Ia) and (IIa) is in its solid form.

The term “pharmaceutically acceptable salt” encompasses salts of the disclosed compounds, including any and all non-toxic pharmaceutically acceptable salts thereof of the disclosed compounds in particular when it refers to a salt of a compound which can serve as an API. Examples of pharmaceutically acceptable addition salts include inorganic and organic acid addition salts and basic salts. The pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; inorganic acid salts such as bromide, chloride, iodide, fluoride, nitrate, hydrochloride, hydrobromide, phosphate, hydrogen phosphate, dihydrogen phosphate, diammonium phosphate, ammoniumhydrogen phosphate, oxalate, perchlorate, sulfate, hydrogen sulfate and the like; organic acid salts such as citrate, lactate, succinate, tosylate, tartrate, maleate, fumarate, mandelate, acetate, dichloroacetate, trifluoroacetate, oxalate, formate and the like; sulfonates such as methanesulfonate, benzenesulfonate, p-toluensulfonate and the like; and amino acid salts such as arginate, asparginate, glutamate and the like.

Acid addition salts can be formed by mixing a solution of the particular compound of the disclosure with a solution of a pharmaceutically acceptable non-toxic acid such as formic acid, sulfuric acid, hydrochloric acid, fumaric acid, maleic acid, succinic acid, acetic acid, citric acid, tartaric acid, carbonic acid, phosphoric acid, oxalic acid, dichloroacetic acid, and the like. Basic salts can be formed by mixing a solution of the particular compound of the disclosure and a pharmaceutically acceptable non-toxic base such as sodium hydroxide, potassium hydroxide, choline hydroxide, sodium carbonate and the like. The term “pharmaceutically acceptable salt” includes anhydrous, solvated, or hydrated forms of the salt.

Whenever a solution or mixture containing a salt is mentioned, the term “pharmaceutically acceptable salt” shall also encompass the dissolved form of the salt. Whenever a 14-hydroxynormorphinone salt is mentioned, this refers to a salt containing a 14-hydroxynormorphinone cation, resulting, e.g., from protonation of the 14-hydroxynormorphinone free base. The same applies to other salts containing a cation with a morphine scaffold. One example of a pharmaceutically acceptable salt of the disclosure is a compound of formula (IIa) (which corresponds to a salt of a compound of formula (II)) or a solvate thereof. An example of such compound of formula (IIa) is a pharmaceutically acceptable salt, e.g., noroxymorphone hydrogen phosphate, which consists of two molecules of noroxymorphone and one molecule of hydrogen phosphate. In this pharmaceutically acceptable salt, the cation results from the protonation of two molecules of noroxymorphone and the anion is the resulting hydrogen phosphate. In preferred embodiments of the disclosure, a pharmaceutically acceptable salt which is a composition comprising compounds of formula (Ia) and (IIa) is in its solid form.

Whenever a compound or formula mentioned in the disclosure contains an atom or structural element which contains one or more asymmetric centers, it may thus give rise to epimers, enantiomers, diastereomers, and stereoisomeric forms. The disclosure is meant to encompass the uses of all such possible forms, as well as their racemic and resolved forms, and mixtures thereof. The individual enantiomers can be separated according to methods known to those of ordinary skill in the art in view of the disclosure. All tautomers are intended to be encompassed by the disclosure as well.

The term “stereoisomer” is a general term for all isomers of individual molecules that differ only in the orientation of their atoms in space. It includes epimers, enantiomers and isomers of compounds with more than one chiral center that are not mirror images of one another (i.e., diastereomers).

The terms “asymmetric center”, “chiral center”, “chiral carbon atom” and “stereocenter” refer to a carbon atom to which four different groups are attached.

The terms “enantiomer” and “enantiomeric” refer to a molecule that cannot be superimposed on its mirror image and hence is optically active where the enantiomer rotates the plane of polarized light in one direction and its mirror image compound rotates the plane of polarized light in the opposite direction.

The term “epimer” refers to two stereoisomers that differ in the configuration of only one stereocenter. All other stereocenters in the molecules, if any, remain the same.

The term “racemic” refers to a mixture of equal parts of enantiomers and which mixture is optically inactive.

The term “resolution” refers to the separation or concentration or depletion of one or two enantiomeric forms of a molecule.

As used herein, the term “molar equivalent” as in, e.g., “amount of acid can be about 10 molar equivalents based on the total molar equivalent of compounds,” refers to the molar amounts of the substances. For example, a molar equivalent of 1 mol of a compound of formula (I) would be 1 mol of H₃PO₄. In another example, 5 molar equivalents of X relative to Y signifies that if 1 mole of Y is used then 5 moles of X are used and 1 molar equivalent of X relative to Y signifies that if 1 mole of Y is used then 1 mole of X is used. In a further example, “10 molar equivalents of H₃PO₄ based on the total molar equivalent of compounds” would be 20 mol of H₃PO₄ when 1.1 mol of a compound of formula (I) is present and 0.9 mol of a compound of formula (II) is present (total molar equivalent of compounds=1.1 mol+0.9 mol).

The term “volumes based on total mass” as in, e.g., “5 volumes based on the total mass of compounds of formulae (I) and (II),” refers to the volume of the first substance determined from total mass of compounds of formulae (I) and (II) present. For example, 2 volumes of X based on the total mass of compounds of formulae (I) and (II) signifies that if 10 g is the total mass of compounds of formulae (I) and (II) present, then 20 cm³ (or 20 mL) of X is used. In another example, 5 volumes of X relative to the mass of Y signifies that if 1 g of Y is used then 5 mL of X are used and 1 volume of X relative to the mass of Y signifies that if 1 g of Y is used then 1 mL of X is used.

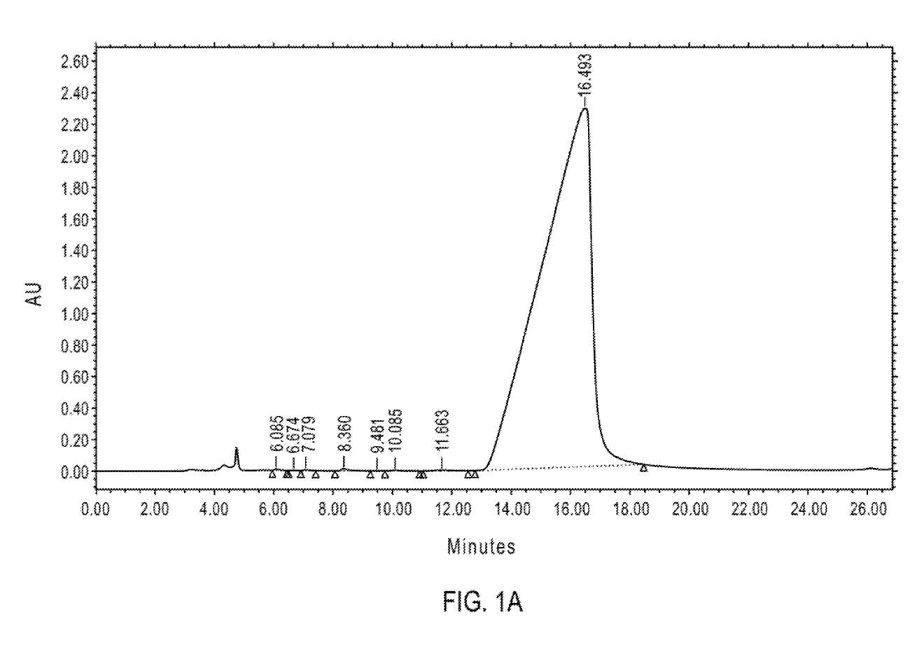

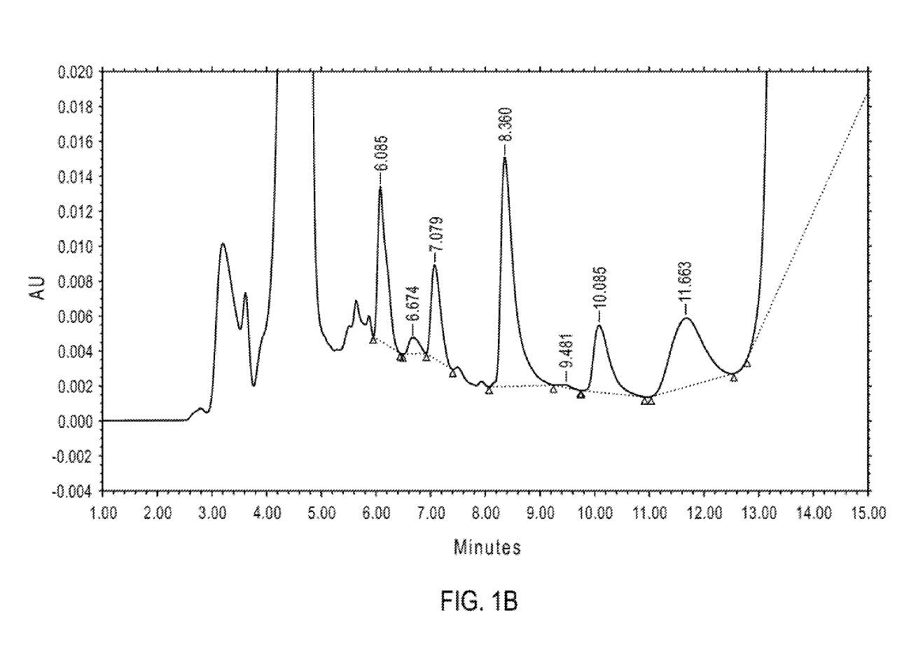

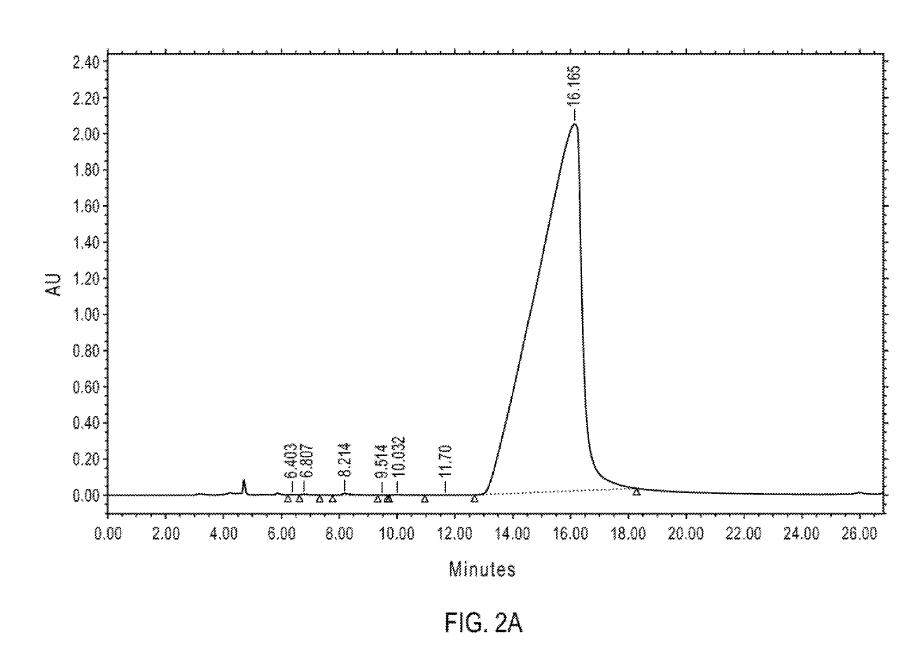

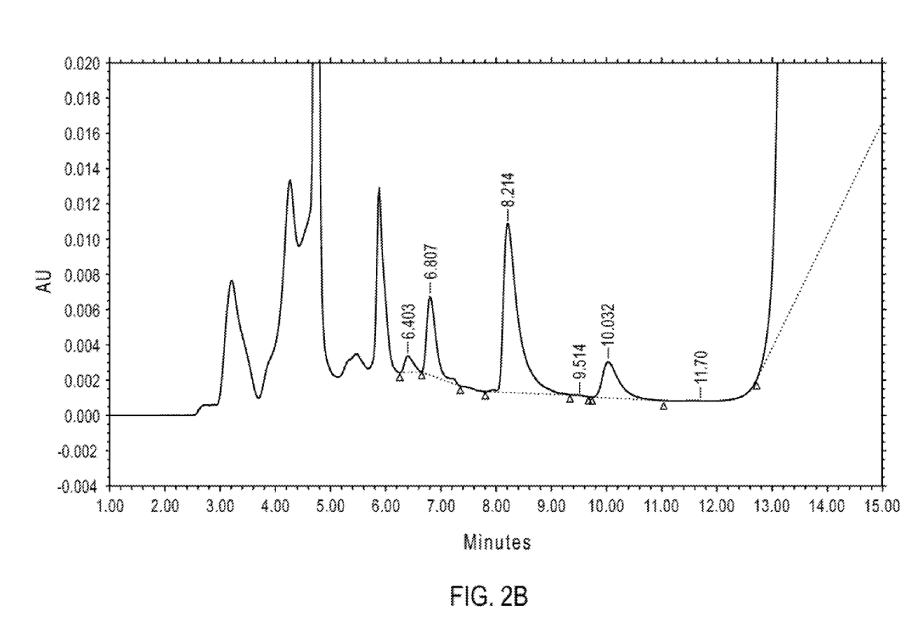

As used in FIGS. 1A, 1B, 2A and 2B herein, the term “AU” means absorbance units.

For morphinan compounds containing the morphine scaffold, the natural stereoconfiguration of the morphine scaffold as shown in the following shall be preferred, where the degree of unsaturation in the ring formed by atoms 5, 6, 7, 8, 13 and 14 can vary. At position 5, the following stereoconfiguration is preferred (exemplified for the morphinan scaffold of formula (I)):

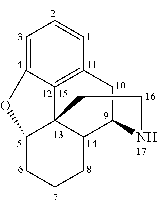 [SMILES: c1cc2c3c(c1)O[C@H]1CCCC4[C@@H](C2)NCC[C@@]341]

For the 8-hydroxy compounds, an α- or a β-configuration is possible at position 8 as illustrated in the following formulae (V) and (VI), respectively:

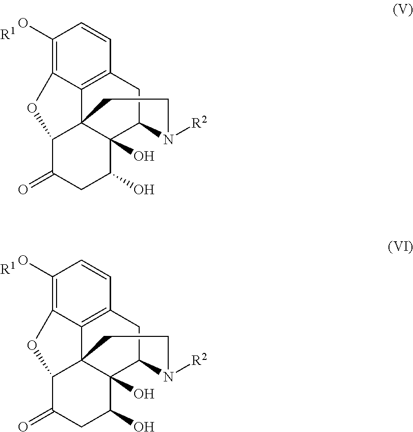

In the compounds and compositions of the disclosure, either both configurations or only one configuration at position 8 may be present. For the compounds of formula (I), the following stereo configuration occurs at position 14 as exemplified for 14-hydroxynormorphinone in the following:

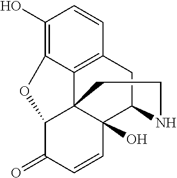 [SMILES: O=C1C=C[C@@]2(O)[C@H]3Cc4ccc(O)c5c4[C@@]2(CCN3)[C@H]1O5]

The term “about” means a value within 15% (±15%) of the value recited immediately after the term “about,” including any numeric value within this range, the value equal to the upper limit (i.e., +15%) and the value equal to the lower limit (i.e., −15%) of this range. For example, the phrase “about 100” encompasses any numeric value that is between 85 and 115, including 85 and 115 (with the exception of “about 100%”, which always has an upper limit of 100%). A further exception is the phrase “about 0” or “about 0%”, which always has a lower limit of 0 or 0%. In certain embodiments, “about” means ±10%, preferably ±5%, more preferably ±1%, or most preferably less than ±1%.

The undefined articles “a,” “an,” and “the” mean one or more of the species designated by the term following said article. For example, “a compound of formula (I)” encompasses one or more molecules of the compound of formula (I).

In the event of doubt as to the agreement of a depicted chemical structure and a chemical name, the chemical name governs.

It is appreciated that various features of the disclosure which are, for clarity, described in the context of separate embodiments, can also be provided in combination in a single embodiment unless otherwise specifically herein excluded. Conversely, various features of the disclosure which are, for brevity, described in the context of a single embodiment, can also be provided separately, in any suitable subcombination, or separately and in any suitable subcombination unless otherwise specifically herein excluded.

The invention includes the following embodiments.

### A. Compounds of Formulae (I), (II), (III) and (IV)

Compounds of formulae (I), (II), (III) and (IV), and salts thereof, which encompass compounds of formula (Ia), (IIa), (IIIa) and (IVa), respectively, and solvates thereof, and compositions of two or more of any of the foregoing compounds are herein disclosed.

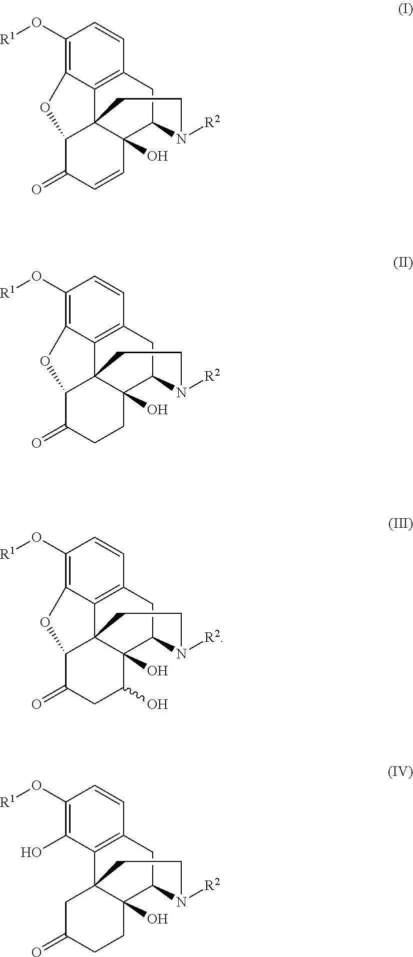

These compounds, and the compositions comprising said compounds or salts or solvates thereof, can be themselves embodiments of the disclosure or can be used as starting materials or intermediates in the process of morphinan derivative synthesis. Unless otherwise indicated, to the compounds of formulae (I), (Ia), (II), (IIa), (III), (IIIa), (IV), (IVa), (V) and (VI), the following applies throughout the specification in connection with each formula reciting R¹, R², or R¹ and R²:
R¹ is —H, —(C₁-C₇)alkyl or an O-protecting group, which O-protecting group, in one embodiment, is selected from the group consisting of acetate, ethyloxycarbonyl, pivolate, benzoate, TBDPS, TMS, TES, TBS, Bn, Tr and tert-butyl; and
R² is —H, —(C₂-C₄)alkenyl, —(C₂-C₇)alkyl, —(C₁-C₇)alkyl-(C₃-C₇)cycloalkyl, —CN, —C(═O)O—(C₁-C₆)alkyl, —C(═O)O-phenyl, or an N-protecting group, which N-protecting group, in one embodiment, is selected from the group consisting of acetamide, ethyloxycarbonyl, Boc, Cbz, Fmoc, Alloc, tosyl, benzenesulfonyl, trifluoromethylcarbonyl, and TroC.

In a preferred embodiment for each formula reciting le unless otherwise indicated, R¹ is —H.

In another embodiment for each formula reciting le unless otherwise indicated, R¹ is —CH₃.

In another embodiment for each formula reciting le unless otherwise indicated, R¹ is an O-protecting group selected from the group consisting of acetate, ethyloxycarbonyl, pivolate, benzoate, TBDPS, TMS, TES, TBS, Bn, Tr and tert-butyl.

In one embodiment for each formula reciting R² unless otherwise indicated, R² is —H.

In another embodiment for each formula reciting R² unless otherwise indicated, R² is —(C₂-C₄)alkenyl.

In another embodiment for each formula reciting R² unless otherwise indicated, R² is —(C₂-C₄)alkenyl or —(C₁-C₇)alkyl-(C₃-C₇)cycloalkyl.

In another embodiment for each formula reciting R² unless otherwise indicated, R² is preferably —CH₂CH═CH₂ or —CH₂-cyclopropyl.

In another embodiment for each formula reciting R² unless otherwise indicated, R² is —CH₂CH═CH₂.

In another embodiment for each formula reciting R² unless otherwise indicated, R² is —CH₂-cyclopropyl.

In another embodiment for each formula reciting R² unless otherwise indicated, R² is a N-protecting group selected from the group consisting of acetamide, ethyloxycarbonyl, Boc, Cbz, Fmoc, Alloc, tosyl, benzenesulfonyl, trifluoromethylcarbonyl, and TroC.

Throughout the specification in connection with each formula reciting Xⁿ⁻, Xⁿ⁻ can be an inorganic anion or organic anion and n is the integer 1, 2, or 3 unless otherwise indicated, and n is preferably 1 or 2.

In another embodiment for each formula reciting n unless otherwise indicated, n is more preferably 1.

In another embodiment for each formula reciting Xⁿ⁻ unless otherwise indicated, Xⁿ⁻ is any anion of a known morphinan derivative salt.

In another embodiment for each formula reciting Xⁿ⁻ unless otherwise indicated, Xⁿ⁻ is selected from Br⁻, Cl⁻, I⁻, F⁻, lactate, NO₃⁻, acetate, tartrate, valerate, citrate, maleate, fumarate, succinate, salicylate, meconate, barbiturate, HSO₄⁻, SO₄²⁻, methanesulfonate, tosylate, trifluoroacetate, H₂PO₄⁻, HPO₄²⁻, [(NH₄)HPO₄]⁻, oxalate, perchlorate, H₃CC(O)O⁻, HC(O)O⁻, and mixtures thereof.

In another embodiment for each formula reciting Xⁿ⁻ unless otherwise indicated, Xⁿ⁻ is preferably selected from the group consisting of Br⁻, Cl⁻, NO₃⁻, HSO₄⁻, SO₄²⁻, tartrate, maleate, fumarate, succinate, citrate, methanesulfonate, tosylate, trifluoroacetate, H₂PO₄⁻, HPO₄²⁻, [(NH₄)HPO₄]⁻, oxalate, perchlorate, H₃CC(O)O⁻, HC(O)O⁻, and mixtures thereof.

In another embodiment for each formula reciting Xⁿ⁻ unless otherwise indicated, Xⁿ⁻ is more preferably selected from the group consisting of HSO₄⁻, SO₄²⁻, H₂PO₄⁻, HPO₄²⁻, [(NH₄)HPO₄]⁻, H₃CC(O)O⁻, HC(O)O⁻, and mixtures thereof.

In another embodiment for each formula reciting Xⁿ⁻ unless otherwise indicated, Xⁿ⁻ is even more preferably selected from the group consisting of H₂PO₄⁻, HPO₄²⁻, [(NH₄)HPO₄]⁻, H₃CC(O)O⁻, HC(O)O⁻, and mixtures thereof.

In another embodiment for each formula reciting Xⁿ⁻ unless otherwise indicated, Xⁿ⁻ is most preferably selected from the group consisting of H₂PO₄⁻, HPO₄²⁻, [(NH₄)HPO₄]⁻, and mixtures thereof.

Any combination of the separate embodiments for the groups R¹, R², Xⁿ⁻ and n is also encompassed in combination in a single embodiment for the compounds of formulae (I), (Ia), (II), (IIa), (III), (IIIa), (IV), (IVa), (V) and (VI) unless otherwise specifically herein excluded.

In all compounds of formulae (I), (Ia), (II), (IIa), (III), (IIIa), (IV), (IVa), (V) and (VI) containing one or more stereocenters, any stereo configuration may be present, unless otherwise indicated. When a compound is the product of a process of the disclosure, those stereocenters of the starting material which are not taking part in the reaction will maintain their stereoconfiguration. In certain embodiments, the stereoconfiguration is described under the Definitions heading herein.

In a preferred embodiment of the processes of the disclosure, the compound of formula (I) is 14-hydroxynormorphinone:

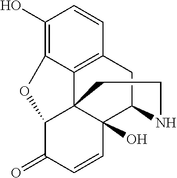 [SMILES: O=C1C=C[C@@]2(O)[C@H]3Cc4ccc(O)c5c4[C@@]2(CCN3)[C@H]1O5]

or a salt or a solvate thereof; and the compound of formula (II) is noroxymorphone:

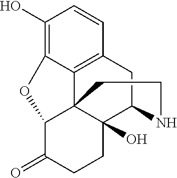 [SMILES: O=C1CC[C@@]2(O)[C@H]3Cc4ccc(O)c5c4[C@@]2(CCN3)[C@H]1O5]

or a salt or a solvate thereof.

In a preferred embodiment, the compound of formula (II) is noroxymorphone; the compound of formula (I) is 14-hydroxynormorphinone; the compound of formula (V) is 8α-hydroxynoroxymorphone, and the compound of formula (VI) is 8β-hydroxynoroxymorphone.

Typically, noroxymorphone is used as a starting material for the synthesis naloxone or naltrexone or a pharmaceutically acceptable salt or solvate thereof.

In certain embodiments, the salt of a compound of formula (I) is a compound of formula (Ia):

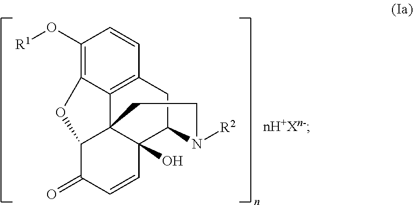

where:
R¹ is —H, (C₁-C₇)alkyl, or an O-protecting group;
R² is —H, —(C₂-C₄)alkenyl, —(C₂-C₇)alkyl, —(C₁-C₇)alkyl-(C₃-C₇)cycloalkyl, —CN, —C(═O)O—(C₁-C₆)alkyl, —C(═O)O-phenyl or an N-protecting group;
Xⁿ⁻ is an anion selected from the group consisting of I⁻, F⁻, valerate, acetate, meconate, salicylate, barbiturate, Br⁻, succinate, tartrate, maleate, fumarate, citrate, NO₃⁻, Cl⁻, HSO₄⁻, SO₄⁻, methanesulfonate, tosylate, trifluoroacetate, H₂PO₄⁻, HPO₄²⁻, [(NH₄)HPO₄]⁻, oxalate, perchlorate, H₃CC(O)O⁻, HC(O)O⁻, and mixtures thereof; and
n is 1 or 2.

In one embodiment, the compound of formula (Ia) is:

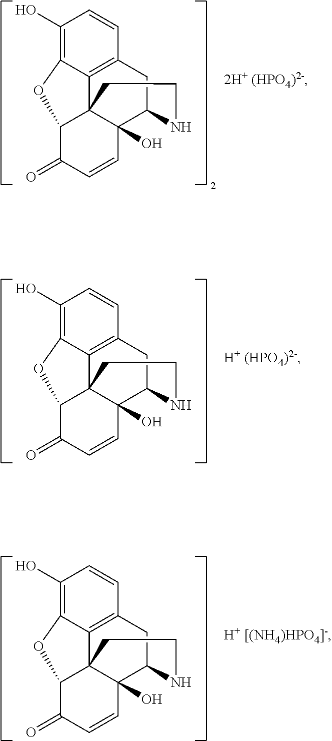

or a solvate (e.g., a hydrate) thereof, or mixtures thereof. These compounds are herein designated as 14-hydroxynormorphinone-(mono)hydrogen phosphate, 14-hydroxynormorphinone-dihydrogen phosphate, and 14-hydroxynormorphinone-ammonium hydrogen phosphate, respectively.

In certain embodiments, the salt of a compound of formula (II) is a compound of formula (IIa):

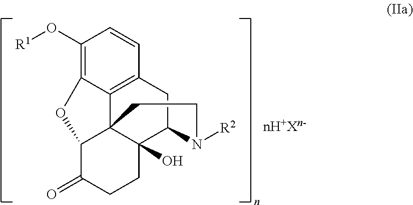

or a solvate thereof; where:
R¹ is —H, (C₁-C₇)alkyl, or an O-protecting group;
R² is —H, —(C₂-C₄)alkenyl, —(C₂-C₇)alkyl, —(C₁-C₇)alkyl-(C₃-C₇)cycloalkyl, —CN, —C(═O)O—(C₁-C₆)alkyl, —C(═O)O-phenyl or an N-protecting group;
Xⁿ⁻ is an anion selected from the group consisting of I⁻, F⁻, valerate, acetate, meconate, salicylate, barbiturate, Br⁻, succinate, tartrate, maleate, fumarate, citrate, NO₃⁻, Cl⁻, HSO₄⁻, SO₄²⁻, methanesulfonate, tosylate, trifluoroacetate, H₂PO₄⁻, HPO₄²⁻, [(NH₄)HPO₄]⁻, oxalate, perchlorate, H₃CC(O)O⁻, HC(O)O⁻, and mixtures thereof; and
n is 1 or 2.

In a preferred embodiment, the compound of formula (IIa) is:

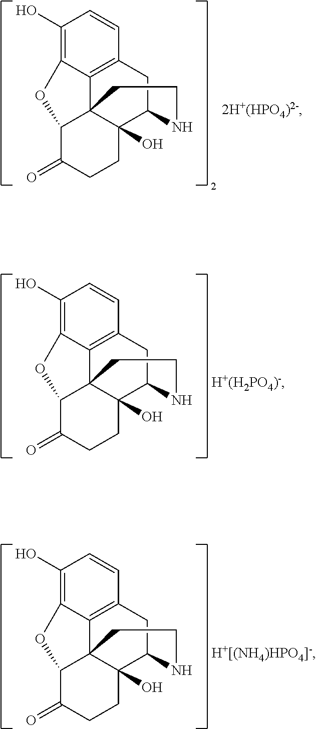

or a solvate (e.g., a hydrate) thereof, or mixtures thereof. These compounds are herein designated as noroxymorphone-(mono)hydrogen phosphate, noroxymorphone-dihydrogen phosphate, and noroxymorphone-ammonium hydrogen phosphate, respectively.

In certain embodiments, the compound of formula (IIa) is:

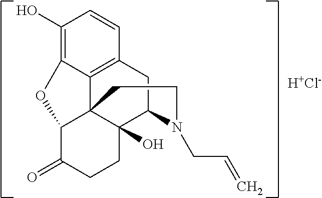

or a solvate (e.g., a hydrate) thereof. This compound is herein designated as naloxone hydrochloride.

In certain embodiments, the compound of formula (IIa) is:

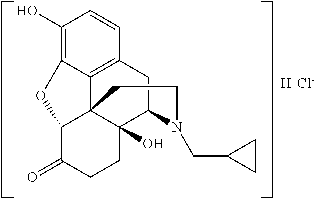

or a solvate (e.g., a hydrate) thereof. This compound is herein designated as naltrexone hydrochloride.

Because of its stoichiometric composition, 14-hydroxynormorphinone-(mono)hydrogen phosphate and noroxymorphone-(mono)hydrogen phosphate can also be designated as bis(14-hydroxynormorphinone)-(mono)hydrogen phosphate and bis(noroxymorphone)-(mono)hydrogen phosphate, respectively, where these terms are used interchangeably herein.

When a solvate of a compound of formulae (I)-(VI) is addressed, it can be any association product of a compound of formulae (I)-(VI) with a solvent molecule. The molar ratio of solvent molecule(s) per molecule of formulae (I)-(VI) can vary. The molar ratio of solvent to compound or a salt thereof in the solvate can be 1 (e.g., in a monohydrate), more than 1 (e.g., 2, 3, 4, 5 or 6 in a polyhydrate), or less than 1 (e.g., 0.5 in a hemihydrate). The molar ratio need not be an integer ratio, it can also be, e.g., 0.5 (as in a hemihydrate) or 2.5. The solvate of the compound of formulae (I)-(VI) is in certain embodiments a hydrate, for example a monohydrate, dihydrate, trihydrate, tetrahydrate, pentahydrate or hexahydrate, or a hydrate where the ratio of water per molecule is not necessarily an integer, but from about 0.5 to about 10.0. In certain embodiments, the solvate of the compound of formulae (I)-(VI) is a hydrate where the ratio of water to compound is from about 1 to about 6. In certain embodiments, the solvate of the compound of formulae (I)-(VI) is a hydrate where the ratio of water to compound is from about 1 to about 3, i.e., a mono- to trihydrate. In certain embodiments, the solvate of the compound of formulae (I)-(VI) is a dihydrate.

### B. Processes for Reducing the Amount of a Compound of Formula (I) in a Composition Comprising Compounds of Formulae (I) and (II)

The disclosure provides a process for reducing the amount of a compound of formula (I) or a salt or a solvate thereof in a composition comprising compounds of formulae (I) and (II) or salts or solvates thereof, the process comprising:
(b) hydrogenating the compound of formula I.

In certain embodiments, hydrogenating step (b) is performed in the presence of an acid H⁺ₙXⁿ⁻, which is added to the reaction composition before or during the hydrogenation reaction of step (b). In a preferred embodiment of the disclosure, the acid H⁺ₙXⁿ⁻ is added before hydrogenating step (b).

The addition of the acid H⁺ₙXⁿ⁻ protonates at least a fraction of the compounds of formula (I), the compounds of formula (II), or a fraction of the compounds of formula (I) and of formula (II) resulting in an increased dissolution of these compounds in the aqueous reaction mixture.

Upon addition of the acid H⁺ₙXⁿ⁻, the compounds of formulae (I) and (II) are protonated and form acid addition salts or solvates thereof, namely compounds of formulae (Ia) and (IIa), namely [(compound (Ia))ₙnH⁺]Xⁿ⁻ and [(compound (IIa))ₙnH⁺]Xⁿ⁻, respectively, as depicted in Scheme 6 below.

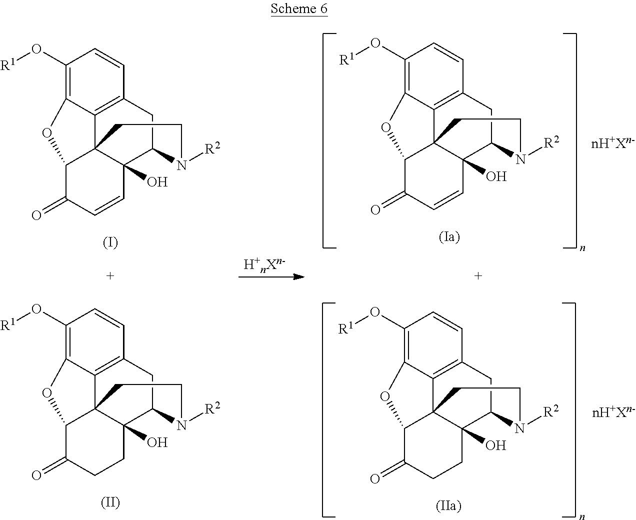

In one embodiment, the acid H⁺ₙXⁿ⁻ is selected from the group consisting of H₂SO₄, H₃PO₄, HC(O)OH, CH₃C(O)OH, and mixtures thereof. In a preferred embodiment, the acid is H₃PO₄. The acids can minimize formation of by-products and can increase the recovery rate of compounds of formula (II) or salts thereof, thereby minimizing the loss of product from the process. Further, these acids can provide improved solubility of the respective salts (compounds of formula (IIa)) during the process in the aqueous mixture of the disclosure, which can be beneficial for the volume efficiency of the inventive process.

In one embodiment, a compound of formula (Ia), a compound of formula (IIa), or a compound of formula (Ia) and a compound of formula (IIa) is formed, where n is 1 and preferably where Xⁿ⁻ is dihydrogen phosphate. In another embodiment, a compound of formula (Ia), a compound of formula (IIa), a compound of formula (Ia) and a compound of formula (IIa), or a solvate thereof is formed, where n is 2 and preferably where Xⁿ⁻ is hydrogen phosphate.

The composition comprising acid addition salts of formulae (Ia) and (IIa) is then subjected to a hydrogenation reaction. During the hydrogenation reaction, the double bond of the ABUK moiety present in compounds of formula (Ia) is hydrogenated, which results in the formation of compounds of formula (IIa).

Therefore, the hydrogenation reaction of the disclosure not only allows for a reduction of the amount of compound of formula (Ia), but also increases the amount of compound of formula (IIa) relative to the original amount of compounds of formula (IIa) present in the initial composition before the hydrogenation reaction.

An exemplary reaction for the hydrogenation of a composition of 14-hydroxynormorphinone (Impurity 1) and noroxymorphone (3), in the presence of hydrogen, a palladium catalyst on carbon and the acid H⁺ₙXⁿ⁻, which is H₃PO₄ in the non-limiting embodiment depicted in the scheme, is shown in Scheme 7 below.

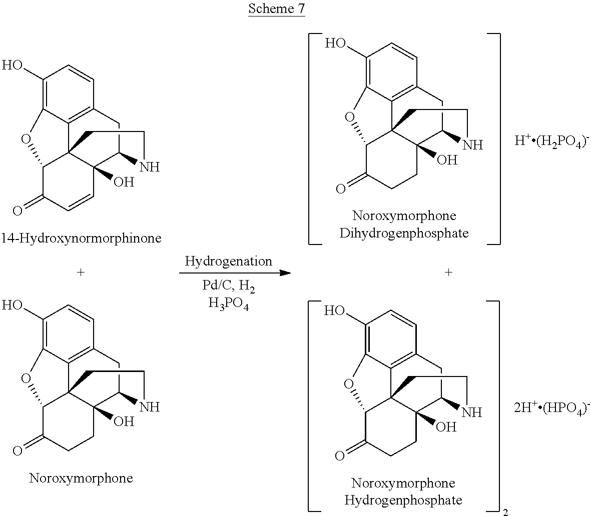

After addition of H₃PO₄, at least a fraction of the noroxymorphone and 14-hydroxynormorphinone is present as acid addition salts comprising noroxymorphone-dihydrogen phosphate, noroxymorphone hydrogen phosphate, 14-hydroxynormorphinone-dihydrogen phosphate, 14-hydroxynormorphinone hydrogen phosphate or any combination thereof.

During the hydrogenation reaction of hydrogenating step (b), the total amount of 14-hydroxynormorphinone in the reaction composition after hydrogenating step (b) is reduced relative to the amount of noroxymorphone present in the reaction composition. 8-Hydroxy compounds of formulae (V) and (VI) or salts or solvates thereof can be present in the composition as further by-products formed during earlier reaction steps and carried through the process. Under acidic conditions, a conversion of compounds of formula (V), and compounds of (VI) possibly to a lesser extent, to a compound of formula (I) can be observed. Since the process of the disclosure is typically performed under acidic conditions and preferably at elevated temperatures, the compounds of formulae (V) and (VI) can be converted as described above and thereby their amount can be reduced compared to their respective amounts in the starting composition.

In certain embodiments, the amount of compounds of formulae (V) and (VI) in the product can be reduced by precipitation of the composition comprising compounds of formulae (I) and (II). This can reduce compounds of formulae (V) and (VI) during subsequent reactions (e.g., during conversion of noroxymorphone to naloxone or naloxone hydrochloride), as compared to reactions which do not involve the step of precipitation.

In certain embodiments, the reaction conditions for hydrogenating step (b) (e.g., time, temperature, relative proportions of the reagents) allow for the formation of a resulting product composition comprising compounds of formulae (I) and (II) or salts or solvates thereof having less than about 200 ppm, less than about 150 ppm, less than about 100 ppm, less than about 75 ppm, less than about 50 ppm, less than about 40 ppm, less than about 35 ppm, less than about 25 ppm, less than about 10 ppm, or less than 5 ppm of a compound of formula (I) or a salt or a solvate thereof relative to the amount of a compound of formula (II) or a salt or a solvate thereof.

B.1. Hydrogenating Step

The hydrogenation reaction of step (b) of the process of the disclosure is represented in Scheme 8 below and comprises the hydrogenating the compound of formula (I) or a salt or a solvate thereof in the inventive composition, thereby forming a compound of formula (II) or a salt or a solvate thereof.

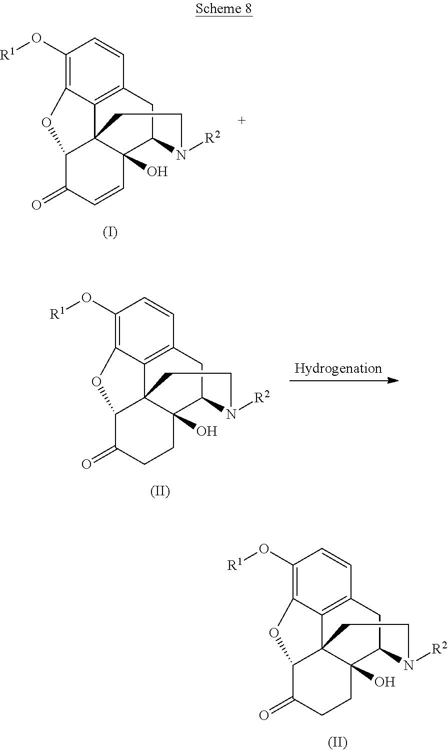

In certain embodiments, the hydrogenation reaction of step (b) generally continues until the amount of the compound of formula (I) or a salt or a solvate thereof relative to the amount of the compound of formula (II) or a salt or a solvate thereof in the composition comprising a compound of formulae (I) and (II) or salts or solvates thereof is less than about 200 ppm, less than about 150 ppm, less than about 100 ppm, less than about 75 ppm, less than about 50 ppm, less than about 40 ppm, less than about 35 ppm, less than about 25 ppm, less than about 10 ppm, or less than 5 ppm.

In a preferred embodiment, the amount of 14-hydroxynormorphinone (a compound of formula (I)) or a salt or a solvate thereof remaining in the composition comprising 14-hydroxynoroxymorphone and noroxymorphone (a compounds of formula (II)) or salts or solvates thereof after the hydrogenation reaction is preferably determined by the HPLC method described in Example 1.1.

The amount of 7,8-didehydronaloxone (a compound of formula (I)) or a salt or a solvate thereof remaining in the composition comprising 7,8-didehydronaloxone and naloxone (a compound of formula (II)) or a salt or a solvate thereof after alkylation of composition comprising 14-hydroxynoroxymorphone and noroxymorphone or salts or solvates thereof is preferably determined by the HPLC method described in Example 1.3.

The amount of 7,8-didehydronaltrexone (a compound of formula (I)) or a salt or a solvate thereof remaining in the composition comprising 7,8-didehydronaltrexone and naltrexone (a compound of formula (II)) or a salt or a solvate thereof after alkylation of composition comprising 14-hydroxynoroxymorphone and noroxymorphone or salts or solvates thereof is preferably determined by the HPLC method described in Example 1.4.

The duration of hydrogenation in step (b) can be from about 1 minute to about 120 hours, from about 1 hour to about 96 hours, from about 2 hours to about 48 hours, from about 2 hours to about 24 hours, or from about 4 hours to about 10 hours. In certain embodiments, the duration of hydrogenation is about 30 minutes, about 1 hour, about 2 hours, about 3 hours, about 4 hours, about 6 hours, about 8 hours, or about 15 hours.

In a preferred embodiment, the duration of hydrogenation in step (b) is from about 1 hour to about 96 hours. In a more preferred embodiment, the duration of hydrogenation is from about 2 hours to about 48 hours. In an even more preferred embodiment, the duration of hydrogenation is from about 4 hours to about 10 hours.

In certain embodiments, the hydrogenating step is terminated when the level of compound of formula (I) or a salt or a solvate thereof in the reaction composition is less than about 200 ppm, less than about 150 ppm, less than about 100 ppm, less than about 75 ppm, less than about 50 ppm, less than about 40 ppm, less than about 35 ppm, less than about 25 ppm, less than about 10 ppm, or less than 5 ppm relative to compounds of formula (II), salts or solvates thereof.

The heterogeneous nature of the reaction composition and different types of reactor vessels used in the process can require variation of reaction times depending on the specific reaction setup in view of the disclosure. The skilled person will be able to adapt the reaction time to the corresponding reaction set up.

The temperature during hydrogenation in step (b) can be anywhere from about 0° C. to about 110° C., from about 10° C. to about 110° C., from about 30° C. to about 90° C., from about 45° C. to about 90° C., or from about 75° C. to about 90° C. In one embodiment, the temperature during hydrogenation is from about 25° C. to about 110° C. In another embodiment, the temperature during hydrogenation is from about 45° C. to about 100° C. In certain embodiments, the temperature during hydrogenation in step (b) is about 25° C., about 35° C., about 50° C., about 65° C., about 70° C., about 75° C., about 80° C., or about 85° C.

In one embodiment, the temperature during hydrogenation in step (b) is from about 75° C. to about 85° C., and the duration of hydrogenation in step (b) is from about 2 hours to about 48 hours. In another embodiment, the temperature during hydrogenation in step (b) is from about 75° C. to about 85° C., and the duration of hydrogenation in step (b) is from about 4 hours to about 10 hours.

During the hydrogenation reaction of step (b), the compound of formula (I) or a salt or a solvate thereof is hydrogenated, thereby forming a compound of formula (II) or a salt or a solvate thereof. Therefore, after completing hydrogenating step (b), the amount of the compound of formula (I) or a salt or a solvate thereof, which is present in the composition comprising compounds of formulae (I) and (II) or salts or solvates thereof, is reduced.

The composition comprising compounds of formulae (I) and (II) can be provided to hydrogenating step (b) in a solution or in a suspension comprising the compounds of formulae (I) and (II) and a suitable solvent. A suitable solvent for the hydrogenation reaction comprises or consists of water, an alcohol (e.g., methanol, ethanol, propanol, iso-propanol, butanol, iso-butanol, tert-butanol, or tert-amyl alcohol), an aromatic hydrocarbon (e.g., benzene, toluene, or xylene), which aromatic hydrocarbon is optionally halogenated (e.g., chlorobenzene, bromobenzene), an aliphatic hydrocarbon (e.g., cyclohexane, cycloheptane), which aliphatic hydrocarbon is optionally substituted (e.g., chloroform, halothan), an ether (e.g., dioxane, tetrahydrofuran, diethylether), a (C₁-C₄)alkyl ester of (C₁-C₄)alkanoic acids (e.g., methyl formate, methyl acetate, or ethyl acetate), an amide (e.g., DMF, diethylformamide, DMAc), other N-(C₁-C₄)alkyl substituted (C₁-C₄)alkanoic acid amides, NMP, formylmorpholine, and mixtures thereof.

In certain embodiments, the solvent for the hydrogenation reaction comprises or consists of water, an ether, a (C₁-C₄)alkane, which alkane is optionally chlorinated, and mixtures thereof. In certain embodiments, the solvent comprises or consists of water, tetrahydrofuran, iso-propanol, methanol, ethanol, butanol, iso-butanol, tert-amylalcohol, n-propanol, chloroform, and mixtures thereof. In certain embodiments, the solvent is iso-propanol or a mixture of iso-propanol and water. In certain other embodiments, the solvent is water. The solvents allow for a volume efficient dissolution of the compounds of formulae (I) and (II).

The ratio of the composition comprising compounds of formulae (I) and (II) to the solvent is selected such that the compounds of formulae (I) and (II) form a suspension or preferably a solution. If excess acid H⁺ₙXⁿ⁻ acts as a solvent, said acid contributes to the total amount of solvent in the reaction mixture or is the sole solvent in the reaction mixture.

In certain embodiments, the volume ratio of the solvent relative to the mass of the composition comprising compounds of formulae (I) and (II) is from about 1 and about 20 volumes. In one embodiment, the volume ratio of the solvent is from about 2 to about 10 volumes. In another embodiment, the volume ratio of the solvent is from about 4 to about 10 volumes. In another embodiment, the volume ratio of the solvent is about 5 volumes.

Typically, the hydrogenation reaction of the composition comprising compounds of formulae (I) and (II) during step (b) is taking place in the presence of a hydrogenation reagent. The compound of formula (I) is then hydrogenated into the compound of formula (II).

In certain embodiments, the hydrogenation reagent can be hydrogen. In one embodiment, the hydrogenation reagent is hydrogen gas, which can be added to the reaction mixture. In one embodiment, the pressure of the hydrogen during hydrogenating step (b) is from about 1×10⁴ Pa to about 200×10⁴ Pa. In other embodiments, the pressure of the hydrogen is from about 15×10⁴ Pa to about 100×10⁴ Pa, from about 30×10⁴ Pa to about 70×10⁴ Pa or from about 45×10⁴ Pa to about 70×10⁴ Pa.

In certain embodiments, the hydrogenation reaction of the composition comprising compounds of formulae (I) and (II) or salts or solvates thereof in hydrogenating step (b) can be performed in the presence of a hydrogenation catalyst. The hydrogenation catalyst is added to the reaction mixture. Typically, the hydrogenation catalyst reduces the temperature and hydrogen pressure required for the hydrogenation reaction, thereby accelerating the reaction. In one embodiment, the hydrogenation catalyst is insoluble.

In certain embodiments, the hydrogenation catalyst is selected as a transition-metal based hydrogenation catalyst. In certain embodiment, the hydrogenation catalyst is selected from the group consisting of rhodium-, ruthenium-, platinum- and palladium-based hydrogenation catalysts, and mixtures thereof. In a preferred embodiment, the hydrogenation catalyst is selected from the group consisting of platinum- and palladium-based hydrogenation catalysts, and mixtures thereof.

In certain embodiments, the hydrogenation catalyst is immobilized on a solid support. In a preferred embodiment the solid support is selected from the group consisting of carbon or BaSO₄, and preferably is carbon.

In one embodiment, the hydrogenation catalyst is selected from the group consisting of palladium on carbon, palladium poisoned with sulfur on carbon (Pd(S)/C), and palladium on BaSO₄. Preferably, the hydrogenation catalyst is palladium on carbon.

In certain embodiments, the hydrogenation catalyst is selected as palladium on carbon, where the palladium is from 1% to 20% on carbon. In certain embodiments, the hydrogenation catalyst is selected from the group consisting of 5% palladium on carbon, 10% palladium on carbon, and mixtures thereof.

Catalysts which can be employed in the disclosed methods include Johnson Matthey (West Deptford, N.J.) catalysts such as 5% Pd/C Types A101002-5, A405028-5, A503023-5 and 5T39, 5% Pd(S)/C Type A103038-5, and 5% Pd/BaSO₄ Types A201053-5 and A308053-5; Evonik Industries (Parsippany, N.J.) catalysts such as 10% Pd/C Type E101 NE/W; or BASF (Iselin, N.J.) catalysts such as 5% Pd/C Types CP-86 EUW, CP-97 EUW, CP-126 EUW, ESCAT™ 143 and ESCAT™ 147.

The amount of hydrogenation catalyst is selected such that said amount is sufficient to catalyze the hydrogenation reaction, i.e., such that the hydrogenation reaction can be performed in the desired reaction time and at a desired reaction temperature without the formation of impurities.

In certain embodiments, the amount of hydrogenation catalyst is from about 0.1 wt % to about 12.0 wt %, from about 1.5 wt % to about 9.0 wt %, from about 1.7 wt % to about 5.0 wt %, or from about 1.8 wt % to about 4.5 wt % based on the total weight of compounds of formulae (I) and (II). In certain embodiments, the amount of hydrogenation catalyst is about 1.8 wt %, about 2.5 wt %, about 5.0 wt %, or about 7.0 wt % based on the total weight of compounds of formulae (I) and (II).

In a preferred embodiment, the amount of hydrogenation catalyst is from about 0.5 wt % to about 12 wt % based on the total weight of compounds of formulae (I) and (II). In another embodiment, the amount of hydrogenation catalyst is from about 1.5 wt % to about 9.4 wt % based on the total weight of compounds of formulae (I) and (II). In another embodiment, the amount of hydrogenation catalyst is from about 1.5 wt % to about 5.0 wt %. In another embodiment, the amount of hydrogenation catalyst is from about 1.5 wt % to about 3.0 wt %. In another embodiment, the amount of hydrogenation catalyst is from about 1.8 wt % to about 2.5 wt %.

In certain embodiments, the hydrogenation catalyst is a 5% palladium on carbon, and the amount of 5% palladium on carbon is at least 0.1 mol% based on the total molar amount of compounds of formulae (I) and (II).

In preferred embodiments, the hydrogenation catalyst is selected as 5% palladium on carbon, and the amount of 5% palladium on carbon is at least about 1.5 wt %, at least about 5 wt % at least about 10 wt % or at least about 15 wt % based on the total weight of compounds of formulae (I) and (II).

In certain embodiments, the hydrogenation catalyst is 10% palladium on carbon, and the amount of 10% palladium on carbon is at least about 0.2 mol% based on the total molar amount of compounds of formulae (I) and (II).

In one embodiment, the hydrogenation catalyst is 10% palladium on carbon, and the amount of 10% palladium on carbon is at least about 1.5 wt % based on the total weight of compounds of formulae (I) and (II). In another embodiment, the amount of 10% palladium on carbon is at least about 3.0 wt %. In another embodiment, the amount of 10% palladium on carbon is at least about 4.0 wt %. In another embodiment, the amount of 10% palladium on carbon is at least about 5.0 wt %.

Before the hydrogenation reaction is initiated (e.g., before adding the hydrogenation reagent, the hydrogenation catalyst, or the hydrogenation reagent and the hydrogenation catalyst), the compound of formula (I) can be present in the reaction composition in any amount. In certain embodiments, the amount of the compound of formula (I) or a salt thereof relative to the amount of the compound of formula (II) or a salt thereof before hydrogenating step (b) is up to about 5000 ppm, up to about 3000 ppm, up to about 2000 ppm, up to about 1500 ppm, up to about 1000 ppm, up to about 500 ppm, or up to about 150 ppm. As the hydrogenation reaction proceeds, the amount of the compound of formula (I) or a salt thereof decreases.

The acid H⁺ₙXⁿ⁻ can be added to the reaction composition of hydrogenating step (b) as acid H⁺ₙXⁿ⁻, or it can be generated in situ in the reaction composition from a salt containing an anion Xⁿ⁻.

The acid H⁺ₙXⁿ⁻ can be added (or generated in situ) before or during the hydrogenation reaction of step (b). The acid can be added once, in several portions or continuously over a certain period of time. It can be added at or during several points in time relative to the hydrogenation reaction, e.g., before or during the hydrogenation reaction. If the acid is added (or generated in situ) before the hydrogenation reaction, during the hydrogenation reaction, or before and during the hydrogenation reaction, the process comprising hydrogenating step (b) can be performed as a one-pot-reaction. Said one-pot-reaction can be more cost-, time- and/or volume-efficient and can therefore be preferred. In a preferred process of the disclosure, the acid H⁺ₙXⁿ⁻ is added to (or generated in situ in) the reaction mixture before the hydrogenation reaction of step (b).

In certain embodiments, a portion or all of the acid H⁺ₙXⁿ⁻ is added before the reaction composition is hydrogenated. In certain embodiments, a portion or all of the acid H⁺ₙXⁿ⁻ is added during the hydrogenation reaction of the composition of the disclosure.

The acid H⁺ₙXⁿ⁻ can be any acid containing an anion Xⁿ⁻ as defined herein. The acid can, for example, be HCl, H₂SO₄ or a HSO₄⁻-salt, methanesulfonic acid, tosylic acid, trifluoroacetic acid, H₃PO₄ or a H₂PO₄⁻-salt, oxalic acid, perchloric acid, HC(O)OH, CH₃C(O)OH, or mixtures thereof.

In one embodiment, the acid H⁺ₙXⁿ⁻ is H₂SO₄, H₃PO₄, HC(O)OH, CH₃C(O)OH, or mixtures thereof. In another embodiment, the acid H⁺ₙXⁿ⁻ is H₃PO₄. The presence of the acid can promote the dissolution of the compounds of formulae (I) and (II) or salts or solvates thereof, and therefore can provide shorter reaction times for the hydrogenation reaction. Moreover, undissolved material of the composition comprising compounds of formulae (I) and (II), or salts or solvates thereof, would tend to reduce the recovery rate, e.g., if the hydrogenation catalyst is separated from the reaction mixture by filtration.

Excess acid can be neutralized during the optional salt-breaking step (c). The more acid that is added, the greater the amount of base that needs to be added to neutralize the reaction composition. The more base added results in more waste generated. From a commercial point of view, excess base usage should therefore be avoided.

If the compound of formula (II) is noroxymorphone, the preferred amount of acid is close to the minimal amount needed to dissolve noroxymorphone. However, a slight excess of acid over this amount (e.g., about a 5% excess) is preferred.

In certain embodiments, the amount of acid H⁺ₙXⁿ⁻ is from about 0.5 to about 10 molar equivalents, from about 1 to about 6 molar equivalents, from about 1.5 to about 4 molar equivalents, or from about 2.2 to about 2.6 molar equivalents based on the total molar equivalents of compounds of formulae (I) and (II).

In one embodiment, the amount of acid is from about 0.5 to about 10 molar equivalents based on the total molar equivalents of compounds of formulae (I) and (II), or (Ia) and (IIa). In another embodiment, the amount of acid is from about 1 to about 6 molar equivalents. In another embodiment, the amount of acid is from about 2 to about 3 molar equivalents. In another embodiment, the amount of acid is from about 2.2 and about 2.6 molar equivalents.

In certain embodiments, the H⁺ provided by H⁺ₙXⁿ⁻ in hydrogenating step (b) is in a molar excess in comparison to the amount of the composition comprising compounds of formulae (I) and (II). In certain embodiments, the molar amount of H⁺ₙXⁿ⁻ present in hydrogenating step (b) is within a range of from about 1.1(1/n) to about 1.2(1/n) molar equivalents per molar equivalent of the composition comprising compounds of formulae (I) and (II), where n is the number of protons in the acid.

In certain embodiments, the acid H⁺ₙXⁿ⁻ is the only acid used during the hydrogenation reaction of step (b). In certain other embodiments, one or more additional acids are added to the reaction mixture in addition to the acid H⁺ₙXⁿ⁻. Said acids can be any selected from the group of acids as defined for the acid H⁺ₙXⁿ⁻ and mixtures of said acids.

The total amount of acid used during hydrogenating step (b) of the process is important, because it can determine if the composition comprising compounds of formulae (I) and (II) is partially or completely dissolved in the process. The total amount of acid can further influence whether or not the composition comprising compounds of formulae (I) and (II) precipitates from the reaction mixture during the process of hydrogenating step (b). The pH of the reaction composition during hydrogenating step (b) is generally acidic (e.g., a pH of less than about 3). It is therefore unexpected that precipitation of the composition comprising compounds of formulae (I) and (II) may occur in the presence of the acid H⁺ₙXⁿ⁻ in the reaction composition. It is assumed that the lower the pH, the more 8α-hydroxy compound of formula (V) converts to the respective ABUK of formula (I) that is then hydrogenated.

In a preferred embodiment, the process for reducing the amount of a 14-hydroxynormorphinone (as a compound of formula (I)) or a salt or a solvate thereof in a composition comprising 14-hydroxynormorphinone and noroxymorphone (as a compound of formula (II)) is performed in the presence of H₃PO₄, and the amount of H₃PO₄ is from about 0.5 to about 10 molar equivalents based on the total molar equivalent of compounds of formulae (I) and (II), or (Ia) and (IIa). In a more preferred embodiment, the amount of H₃PO₄ is from about 1 to about 6 molar equivalents. In a most preferred embodiment, the amount of H₃PO₄ is from about 2.2 to about 2.6 molar equivalents.

As an alternative to adding H⁺ₙXⁿ⁻ to the reaction composition, the acid H⁺ₙXⁿ⁻ can be generated by adding a salt containing Xⁿ⁻ to the reaction composition. Said salt can have the formula

M^{m+}(H⁺)₍ₙ₋ₘ₎Xⁿ⁻ or M^{m+}_{((n-q)/m)}(H⁺)_{q}Hⁿ⁻;

where:
M^{m+} is a monovalent or polyvalent metal cation;
m and n are independent from each other and an integer selected from 1, 2, or 3, provided that m is ≦n; and
q is an integer selected from 0, 1, and 2, provided that q<n.

The metal cation M can be an alkali metal cation, an alkaline earth metal cation or a group III cation. Exemplary cations include Na⁺, K⁺ and Ca²⁺. Exemplary salts, in one embodiment, are selected from the group consisting of NaHSO₄, KHSO₄, Na₂SO₄, K₂SO₄, NaH₂PO₄, Na₂HPO₄, Na₃PO₄, KH₂PO₄, K₂HPO₄, and K₃PO₄.

As a further alternative to adding an acid H⁺ₙXⁿ⁻ in hydrogenating step (b), a Lewis acid can be added to the reaction composition instead of the acid H⁺ₙXⁿ⁻. Such non-limiting Lewis acids can be aluminum chloride (AlCl₃), aluminum bromide (AlBr₃), boron trifluoride (BF₃), boron trifluoride diethyl etherate (BF₃.Et₂O), iron(III) chloride (FeCl₃), and the like.

During hydrogenating step (b), a ring opening reaction at position 5 of the compound of formula (II) or a salt or a solvate thereof can be observed, resulting in the formation of additional by-products such as compounds of formula (IV) or a salt or a solvate thereof, as depicted in Scheme 9 below. The by-product results in a lower yield of compound of formula (II), and therefore ring opening is not desired.

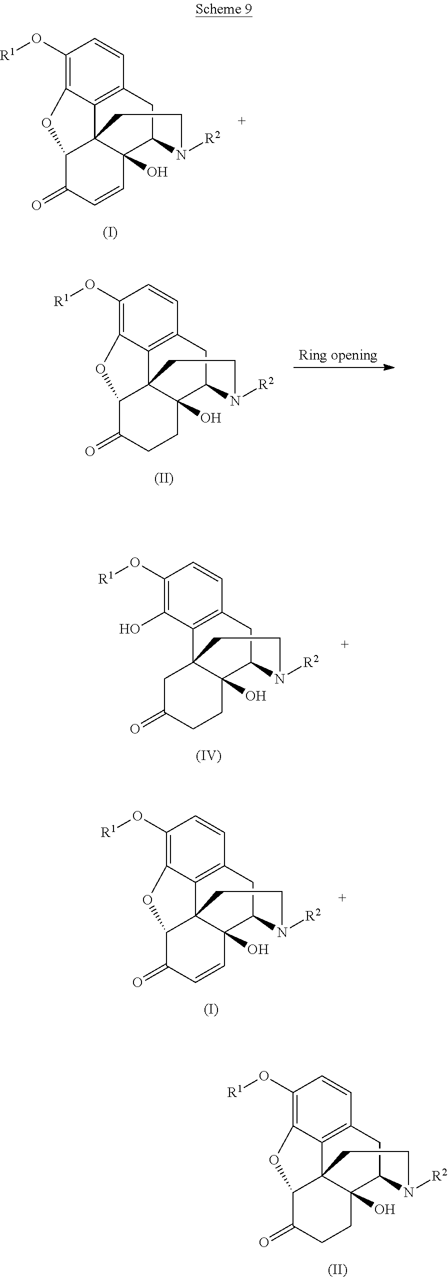

where, for compounds of formula (IV):
R¹ is —H, (C₁-C₇)alkyl, or an O-protecting group; and
R² is —H, —(C₂-C₄)alkenyl, —(C₂-C₇)alkyl, —(C₁-C₇)alkyl-(C₃-C₇)cycloalkyl, —CN, —C(═O)—(C₁-C₆)alkyl, —C(═O)O-phenyl or an N-protecting group.

The formation of compounds of formula (IV) can be suppressed by addition of an additive to the reaction composition comprising compounds of formulae (I) and (II) or salts or solvates thereof. The additive can be a halide-containing compound. Therefore, the amount of the compound of formula (IV) or a salt or a solvate thereof in the composition comprising compounds of formulae (I) and (II) or a salt or a solvate thereof may be reduced or completely suppressed by addition of an additive to the reaction composition. In certain embodiments, the halide-containing compound is selected from the group consisting of a halide salt or halide forming compounds. In one embodiment, the halide of the halide-containing compound is chloride or iodide, and preferably iodide. Preferably, the halide-containing compound is selected from the group consisting of ammonium chloride, sodium iodide, sodium chloride, sodium bromide and hydrochloric acid.

In one embodiment, the amount of the halide-containing compound is from about 0.0001 wt % to about 15.0 wt % based on the total weight of compounds of formulae (I) and (II) or salts or solvates thereof. In another embodiment, the amount of halide-containing compound is from about 1.0 wt % to about 12.0 wt %. In another embodiment, the amount of halide-containing compound is from about 2.5 wt % to about 10.0 wt %. In another embodiment, the amount of halide-containing compound is from about 3.5 wt % to about 7.5 wt %. In another embodiment, the amount of halide-containing compound is from about 4.5 wt % and about 5.0 wt %.

In one embodiment, the halide-containing compound is an iodide-containing compound. The amount of iodide-containing compound, in certain embodiments, is from about 0.0001 wt % to about 15 wt %, from about 0.0005 wt % to about 5 wt %, from about 0.001 wt % to about 1 wt %, from about 0.002 wt % to about 0.5 wt %, or from about 0.0025 wt % to about 0.1 wt % based on the total weight of compounds of formulae (I) and (II). Preferably, the iodide-containing compound is sodium iodide.

In certain embodiments, the halide-containing compound is sodium iodide, and the amount of sodium iodide is less than 250 ppm, less than 500 ppm, or less than 1000 ppm based on the total weight of compounds of formulae (I) and (II).

In certain embodiments, the halide-containing compound is a chloride-containing compound. The amount of chloride-containing compound, in certain embodiments, is from about 0.5 wt % to about 15.0 wt %, from about 1.0 wt % to about 12.0 wt %, from about 2.5 wt % to about 10.0 wt %, from about 3.5 wt % to about 7.5 wt %, or from about 4.5 wt % to about 5.5 wt % based on the total weight of compounds of formulae (I) and (II). Preferably, the chloride-containing compound is sodium chloride.

In certain embodiments, the addition of the halide-containing compound is performed before the addition of the hydrogenation catalyst.

In certain embodiments, the addition of halide-containing compound is performed before hydrogenating step (b), during hydrogenating step (b), or before and during hydrogenating step (b).

After completion of the hydrogenation reaction, the hydrogenation catalyst can be removed from the reaction mixture by filtration. To minimize the loss of the composition comprising compounds of formulae (I) and (II), or the salts or solvates thereof, the filtered hydrogenation catalyst can be washed with a washing solvent. In one embodiment, the washing solvent is selected from the group consisting of water, methanol, ethanol, iso-propanol, an acid (such as H₂SO₄, H₃PO₄, HC(O)OH, or CH₃C(O)OH), and mixtures thereof. In another embodiment, the washing solvent is water.

In one embodiment, the amount of solvent used for washing the filtered hydrogenation catalyst is from about 0.5 to about 10 volumes based on the total mass of compounds of formulae (I) and (II). In another embodiment, the amount of solvent used is from about 1.0 to about 4 volumes. In another embodiment, the amount of solvent used is from about 1 volume to about 3 volumes. If required, the filtered hydrogen catalyst can be washed more than once.

The hydrogenation reaction can be prepared in any suitable reaction vessel. In one embodiment, the reaction vessel is a flow reactor. In another embodiment, the reaction vessel is a continuous flow reactor. In another embodiment, the reaction vessel is not a continuous flow reactor. In another embodiment, the reaction vessel is not a flow reactor.

B.2. Salt-Breaking Step

The process of the disclosure can further comprise an optional salt-breaking step (c), which comprises the addition of a base after hydrogenating step (b).

Since hydrogenating step (b) typically takes place under acidic conditions, the compounds of formulae (I) and (II) of the reaction composition during hydrogenating step (b), after hydrogenating step (b), or during and after hydrogenating step (b) are at least partially present as the corresponding salts thereof, namely as compounds of formulae (Ia) and (IIa) or a solvate thereof, respectively.

The salt-breaking step (c) of the process of the disclosure is initiated by addition of a base as summarized in Scheme 10 below. Upon base addition during salt-breaking step (c), the salts of formulae (Ia) and (IIa) or solvates thereof are converted to compounds of formulae (I) and (II) or solvates thereof.

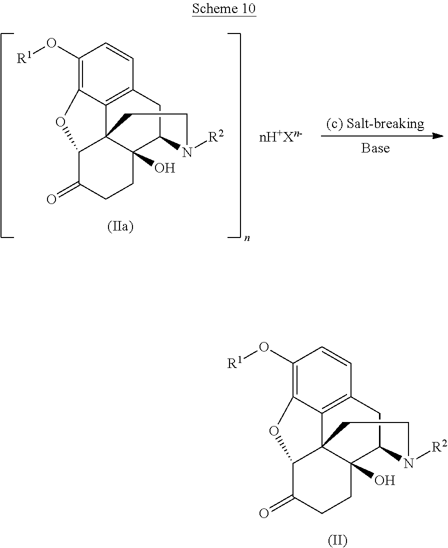

The salt-breaking reaction of step (c), according to the process of the disclosure, can also encompass precipitation, crystallization, or precipitation and crystallization of the composition, thereby yielding compounds of formulae (I) and (II) or a solvate thereof in solid form.

The base can comprise an inorganic base or an organic base. In one embodiment, the base is selected from the group consisting of sodium hydroxide, potassium hydroxide, aluminum hydroxide, ammonia, ammonium hydroxide and other organic bases, e.g., pyridine, methyl amine, imidazole, benzimidazole or histidine. In a preferred embodiment, the base is ammonium hydroxide.

In another embodiment, the base is another organic base. The organic base is preferably selected from the group consisting of pyridine, methyl amine, imidazole, benzimidazole, histidine and phosphazene bases.

In another embodiment, the ratio of the amount of compound of formula (I), which is present in the composition comprising compounds of formulae (I) and (II) after hydrogenating step (b), to the compound of formula (II) is maintained or even further reduced during the salt-breaking step (c).

In a preferred embodiment of the salt-breaking step (c), the addition of the base is performed under controlled conditions. One of these conditions that is controlled is the pH-value after addition of the base. Another condition that is controlled is temperature.

Typically, in the process of the disclosure, the end point of base addition (i.e., the neutralization reaction) in the salt-breaking reaction of step (c) is determined by the pH-value of the reaction mixture after base addition. The resulting pH-value can be from about 7.0 to about 12.0, from about 7.5 to about 9.5, or from about 8.0 to about 9.0. In one embodiment, the pH value at the end point of the neutralization is from about 7.0 to about 9.0.

The base can be added to the reaction composition during salt-breaking step (c) in one or more portions. In other words, this means that the total amount of the base may not be exclusively provided in a single portion, but instead may be split into a plurality of portions, which can then be added separately to the reaction composition. The subsequent base addition steps can be performed, e.g., at the same temperature compared to the first base addition step or at different temperatures. In certain embodiments, the base is added in 2, 3, 4 or 5 portions. In a preferred embodiment, the base is added in 1 or 2 portions. In a more preferred embodiment, the base is added in 2 portions.

In an alternative embodiment, the base is added in one portion and the temperature is increased over time.

The temperature has an influence on the stability of the compound of formula (II) or a salt or a solvate thereof. Therefore, the temperature during the base addition influences the amount of compound of formula (II) or a salt or a solvate thereof in the composition. During base addition, in certain embodiment the temperature of the product of hydrogenating step (b) when at least a first portion of the base in salt-breaking step (c) is added thereto can be from about 0° C. to about 100° C., from about 30° C. to about 100° C., from about 40° C. to about 90° C., from about 50° C. to about 90° C., from about 60° C. to about 90° C., or from about 70° C. to about 80° C.

In certain embodiments, during base addition the temperature of the product of hydrogenating step (b) when at least a first portion of the base in salt-breaking step (c) is added thereto is about 10° C., about 15° C., about 20° C., about 25° C., about 30° C., about 35° C., or about 40° C. Optionally, the temperature can be changed during base addition.

At least a first portion of the base in salt-breaking step (c) is added to the product of hydrogenating step (b) until the pH, in certain embodiments, is from about 2.0 to about 7.0, from about 2.0 to about 6.0, from about 3.5 to about 5.5, from about 4.0 to about 5.5, or from about 4.5 to about 5.5. In certain embodiments, the pH after addition of at least the first portion of base is about 3.0, about 4.0, about 5.0, about 6.0 or about 7.0.

In one embodiment, at least a first portion of the base in salt-breaking step (c) is added to the product of hydrogenating step (b) until the pH is from about 2.0 to about 6.0. In a more preferred embodiment, the base is added until the pH is from about 4.5 to about 5.5. In a most preferred embodiment, the base is added until the pH is about 5.0.

In certain embodiments, during base addition the temperature when the at least first portion of the base in salt-breaking step (c) is added thereto can be anywhere from about 0° C. to about 90° C., from about 5° C. to about 70° C., from about 10° C. to about 60° C., from about 15° C. to about 50° C., from about 20° C. to about 40° C., or from about 20° C. to about 30° C.

In preferred embodiments, during base addition the temperature when the at least first portion of the base in salt-breaking step (c) is added thereto is from about 15° C. to about 50° C., more preferably from about 20° C. to about 30° C. or about 25° C.

In certain embodiments, the base is added in salt-breaking step (c) in one portion.

In certain embodiments, the base is added in salt-breaking step (c) in at least two portions.

In certain embodiments, where the base is added in two portions in salt-breaking step (c), a first portion of the base is added to the product of hydrogenating step (b) while the temperature is from about 15° C. to about 50° C. or from about 20° C. to about 30° C., until the pH is from about 2.0 to about 6.0.

In certain embodiments, where the base is added in two portions in salt-breaking step (c), a first portion of the base is added to the product of hydrogenating step (b) while the temperature is from about 15° C. to about 50° C. or from about 20° C. to about 30° C., until the pH is from about 4.5 to about 5.5.

In preferred embodiments, where the base is added in two portions in salt-breaking step (c), a second portion of the base is added while the temperature is from about 40° C. to about 90° C. or from about 70° C. to about 80° C., until a pH of from about 7.0 to about 9.0 is reached.

In preferred embodiments, where the base is added in two portions in salt-breaking step (c), a second portion of the base is added while the temperature is from about 40° C. to about 90° C., from about 70° C. to about 80° C., or about 75° C., until a pH of from about 7.5 and about 8.5 is reached.

In one embodiment, where the base is added in two portions in salt-breaking step (c), a first portion of the base is added to the product of hydrogenating step (b) while the temperature is from about 20° C. to about 30° C., until the pH is from about 4.5 to about 5.5, and the temperature when a second portion of the base in salt-breaking step (c) is added thereto is from about 70° C. to about 80° C., until a pH of from about 7.5 to about 8.5 is reached. In a preferred embodiment, the compound of formula (II) is noroxymorphone, which can be isolated after salt-breaking salt-breaking step (c), e.g., as anhydrous or dihydrate, or mixtures thereof, and preferably is isolated as anhydrous.

In certain embodiments, where the base is added in two portions in salt-breaking step (c), a first portion of the base is added to the product of hydrogenating step (b) while the temperature is from about 15° C. to about 50° C. or from about 20° C. to about 30° C., until the pH is from about 2.0 to about 6.0, preferably from about 4.5 to about 5.5 and most preferably about 5.0, and the temperature when a second portion of the base in salt-breaking step (c) is added thereto is from about 40° C. to about 90° C., preferably from about 70° C. to about 80° C. and most preferably about 75° C., until a pH of from about 7.0 to about 9.5 is reached.

Typically, the composition comprising a salt of compounds of formulae (I) and (II) thereof will shift directly from hydrogenating step (b) to salt-breaking step (c) without physical isolation of the step (b) product from the reaction mixture.

In certain embodiments, the composition comprising a salt of compounds of formulae (I) and (II) can be isolated after hydrogenating step (b) and before it is transferred to the salt-breaking step (c). For example, isolation of the salt, as a precipitate, can occur after addition of an anti-solvent. The compounds of formulae (I) and (II) of the isolated precipitate after antisolvent addition can then be redissolved and isolated as free base in salt-breaking step (c). Typically, the composition comprising a salt of compounds of formulae (I) and (II) can be provided for salt-breaking step (c) in a solution or suspension comprising the salt of compounds of formulae (I) and (II) and a suitable solvent.

Suitable solvents for salt-breaking step (c) are selected from the group consisting of water, alcohols (e.g., methanol, ethanol, propanol, iso-propanol, butanol, iso-butanol, tert-butanol, tert-amyl alcohol), aromatic hydrocarbons (e.g., benzene, toluene, xylene), which aromatic hydrocarbons are optionally halogenated (e.g., chlorobenzene, bromobenzene), aliphatic hydrocarbons (e.g., hexane, heptane, cyclohexane, cycloheptane), which aliphatic hydrocarbons are optionally substituted (e.g., chloroform, halothan), ethers (e.g., dioxane, tetrahydrofuran, diethylether), (C₁-C₄)alkyl esters of (C₁-C₄)alkanoic acids (e.g., methyl formate, methyl acetate, ethyl acetate), amides (e.g., DMF, diethylformamide, DMAc), other N-(C₁-C₄)alkyl substituted (C₁-C₄)alkanoic acid amides, NMP, formylmorpholine, and mixtures thereof.

In certain embodiments during, after or during and after the adjustment of the pH-value (e.g., by addition of the base), the composition comprising compounds of formulae (I) and (II) is precipitated from the reaction mixture.

Alternatively, in addition to the presence of the base, precipitation can be started or enhanced by other measures, e.g., by adjusting the temperature of the solution, by adding an anti-solvent to the solution, or by adjusting the temperature of the solution and by adding an anti-solvent to the solution, as described in more detail below. In certain embodiments, precipitation can be achieved by adding an antisolvent. In certain embodiments, precipitation can be achieved by lowering the temperature. The pH of the reaction mixture at this point is generally basic (e.g., a pH of about 8). It is therefore expected that in the presence of a base in the reaction mixture, precipitation of the composition comprising compounds of formulae (I) and (II) or a solvate thereof can take place.

After salt-breaking step (c), the reaction composition can be filtered to isolate the product in an isolating step (d).

In certain embodiments, the temperature of the reaction composition before filtration but after salt-breaking step (c) is from about 20° C. to about 80° C., from about 30° C. to about 70° C. or from about 40° C. to about 60° C.

In one preferred embodiment, the temperature of the reaction composition after salt-breaking step (c) but before filtration is from about 5° C. to about 90° C., from about 20° C. to about 70° C., from about 40° C. to about 50° C. or about 45° C.

In certain embodiments, the amount of washing solvent used for washing the filtered hydrogenation catalyst is from about 0.1 to about 12 volumes based on the total mass of the filtered hydrogenation catalyst, from about 0.5 volumes to about 8 volumes, from about 1 volume to about 4 volumes, or about 2 volumes.

In certain embodiments, the temperature after filtration is lowered to less than 55° C. In certain embodiments, the composition comprising compounds of formulae (I) and (II) can be isolated from the reaction mixture after salt-breaking, thereby comprising isolating step (d).

In said isolating step (d), the composition comprising compounds of formulae (I) and (II) or salts or solvates thereof can be separated from the supernatant in any conventional manner, e.g., by filtration, centrifugation, decanting, precipitation or any other conventional method for separating a solid phase from a liquid phase.

The isolated composition comprising compounds of formulae (I) and (II) or solvates thereof is preferably dried after isolation. It has been found that by varying certain parameters, such as temperature, during the drying step, a composition or a solvate thereof can be obtained which is less susceptible to hygroscopic uptake of water after isolation and thereby yields a more stable product.

In certain embodiments, the drying step is performed under reduced pressure. In certain embodiments, the drying step is performed under an inert gas atmosphere. In one embodiment, the drying step is performed under a nitrogen gas atmosphere.

In certain embodiments, the drying step is performed with a gas pressure of less than about 10×10⁴ Pa, less than about 7×10⁴ Pa or less than about 3.5×10⁴ Pa.

The drying time can be from about 1 minute and 120 hours. In one embodiment, the drying step is performed over a time period of from about 1 hour to about 48 hours. In other embodiments, the drying step is performed from about 8 hours to about 24 hours or from about 12 hours to about 20 hours.

In certain embodiments, if may be beneficial to perform the drying step at an elevated temperature. In certain embodiments, the drying temperature is from about 20° C. to about 110° C. or from about 40° C. to about 100° C.

In one embodiment, the drying step is performed at a temperature from about 10° C. to about 120° C. In other embodiments, the drying step is performed at a temperature from about 40° C. to about 100° C. or from about 80° C. to about 90° C. In one embodiment, the drying step is performed at the indicated temperatures (e.g., from about 80° C. to about 100° C.) at a reduced pressure.

Typically, when the reaction composition is dried at a temperature of about 60° C. to about 100° C. at reduced pressure and preferably at about 80° C., the reaction composition can be efficiently dehydrated and the resulting product is non-hydroscopic, i.e., it does not adsorb more than 1 wt % water upon storage at relative humidities up to 80%.

Typically, when the reaction composition is dried at a temperature of less than 50° C., the reaction composition may not be efficiently dehydrated and the resulting product can be hydroscopic. Therefore said reaction composition can absorb water during storage over time.

In one embodiment, the drying step is performed until the water content of the composition of compounds of formulae (I) and (II) or the salts or solvates thereof is less than about 20 wt % based on the total weight of compounds of formulae (I) and (II) or the salts or solvates thereof. In other embodiments, the water content is less than about 13 wt %, less than about 11 wt %, less than about 5 wt % or less than about 1 wt %.

Precipitation can start as soon as a base is added to the reaction mixture (e.g., upon addition of ammonium hydroxide) or it can start later, e.g., at a certain pH-value. In certain embodiments where the composition comprising salts of compounds of formulae (I) and (II), or solvates thereof, precipitates from the reaction mixture, the base is sodium hydroxide, potassium hydroxide, aluminum hydroxide, ammonia, ammonium hydroxide, or mixtures thereof. In one embodiment, the base is ammonium hydroxide.

In certain embodiments, the precipitation of the composition comprising a salt of compounds of formulae (I) and (II) or the solvates thereof can require cooling of the reaction mixture, the addition of an anti-solvent, or cooling of the reaction mixture and the addition of an anti-solvent.

The composition comprising compounds of formulae (I) and (II) or the solvate thereof, once precipitated, can preferably be isolated (i.e., separated from the reaction mixture). Alternatively, the composition comprising compounds of formulae (I) and (II) can be converted to further compounds, e.g., to naloxone, naltrexone or salts or solvates thereof, during a subsequent synthesis step without the need for isolation or further purification.

Once precipitated, the precipitate containing the composition comprising compounds of formulae (I) and (II) or solvates thereof can optionally be subjected to one or more steps to further reduce the amount of compounds of formula (I) therein. These further steps can include crystallization, recrystallization or heat treatment and are described in the subsequent section.

Precipitation of the composition comprising compounds of formulae (I) and (II) and the formation of a specific form can be influenced by various parameters. These parameters can include:

the pH-value of the reaction mixture,

the temperature before, during, or after salt-breaking step (c),

the amount of solvent present in the reaction composition,

the optional presence of an anti-solvent added to the reaction composition,

the rate at which the base is added to the reaction mixture during the process, or

by a combination of any of the foregoing.

In one embodiment, precipitation of the composition comprising compounds of formulae (I) and (II) or solvates thereof is initiated by, enhanced by, or initiated and enhanced by one or more of the following:
(i) adjusting (e.g., lowering) the temperature of the reaction composition,
(ii) adding an anti-solvent to the reaction composition,
(iii) adding a seed crystal to the reaction composition,
(iv) adjusting (e.g., lowering or increasing) the pH of the reaction composition,
(v) changing the ionic strength of the reaction composition (e.g., by adding a salt to the reaction composition),
(vi) concentrating the reaction composition,
(vii) reducing or stopping agitation of the reaction composition,
   or any other conventional method for initiating or enhancing precipitation or crystallization.

In one embodiment, the precipitation of the composition comprising compounds of formulae (I) and (II) or a solvate thereof is initiated by, enhanced by, or initiated and enhanced by adjusting (e.g., increasing) the pH of the reaction composition.

When the temperature is adjusted to the precipitation temperature, this means that precipitation of the composition comprising compounds of formulae (I) and (II) or the solvates thereof is initiated by, enhanced by, or initiated and enhanced by adjusting the temperature of the reaction mixture to or below a temperature at which said compound begins to precipitate (i.e., the “precipitation temperature”). The temperature is either adjusted by performing salt-breaking step (c) at the precipitation temperature, or by lowering the temperature of the reaction mixture during, after, or during and after completion of the reaction.

In certain embodiments, precipitation of the composition comprising compounds of formulae (I) and (II) or solvates thereof is performed at a temperature of from about 60° C. to about 90° C. or from about 70° C. to about 80° C. The reaction mixture comprising the precipitate is then cooled to about 45° C. and the precipitate is isolated from the reaction mixture, e.g., by filtration.

In certain embodiments, an anti-solvent is added to precipitate the composition comprising compounds of formulae (I) and (II) or a solvate thereof. When an anti-solvent is added to the reaction mixture, it is added either during or after salt-breaking step (c) and in an amount effective to initiate, enhance, or initiate and enhance precipitation. In certain embodiments, addition of a suitable anti-solvent increases the yield of the reaction. A suitable anti-solvent can comprise or consist of tert-butyl methyl ether, diethyl ether, hexane(s), tert-amyl alcohol, methanol, ethanol, isopropyl alcohol, 2-butanol, heptanes, xylenes, toluene, acetone, 2-butanone, ethyl acetate, tetrahydrofuran, 1,2-dichloromethane, chloroform, dichloromethane, or mixtures thereof. 14-Hydroxynormorphinone and noroxymorphone bases, according to the disclosure, have very low or no solubility in these solvents at a temperature of about 25° C. In another embodiment, said anti-solvent is an alcohol, e.g., methanol. In certain embodiment, said anti-solvent is an ether, e.g., tert-butyl methyl ether. In certain embodiment, said anti-solvent is a mixture of an alcohol (e.g., methanol) and an ether (e.g., tert-butyl methyl ether), for example a mixture of methanol and tert-butyl methyl ether. When two or more anti-solvents are used (e.g., in a mixture), the solvents can be added as a mixture or separately.

When a seed crystal is added, said seed crystal is a crystal of the composition comprising compounds of formula (I), compounds of formula (II), compounds of formulae (I)and (II), or a solvate thereof. This seed crystal can act as a crystallization nucleus if the solution of the composition comprising compounds of formulae (I) and (II) resulting from salt-breaking step (c) is metastable; it can be made metastable by adjusting the concentration of the reaction mixture, by adjusting the temperature, by adjusting the solvent composition or by any combination thereof.

B.3. Decolorizing Step

If the composition comprising compounds of formulae (I) and (II) or salts or solvates thereof has color, the reduction or removal of said color is intended to improve the appearance of the product.

The process of the disclosure further comprises an optional decolorizing step (a), which comprises the addition of a decolorizing agent to the composition comprising compounds of formulae (I) and (II) or a salt or a solvate thereof.

In certain embodiments, decolorizing step (a) is performed by at least one of before, during, or after hydrogenating step (b).

In certain embodiments, decolorizing step (a) is performed by at least one of before, during, or after salt-breaking step (c).

In certain embodiments, decolorizing step (a) and hydrogenating step (b) are performed simultaneously.

In a preferred embodiment, decolorizing step (a) is performed before hydrogenating step (b).

Isolation of the composition comprising compounds of formulae (I) and (II) or salts or solvates thereof prepared by conventional processes can result in a colored product, e.g., with a brown or dark yellow color. Therefore, an additional object of the process of the disclosure is to partially or completely remove the color from the isolated composition comprising compounds of formulae (I) and (II) or salts or solvates thereof.

In certain embodiments, decolorizing step (a) is performed until the transparency of the solution of the reaction composition is improved.

In one embodiment, decolorizing step (a) is performed until the color of the solution of the reaction composition is dark yellow, yellow or light yellow.

In a more preferred embodiment, decolorizing step (a) is performed until the color of the isolated reaction composition product is light yellow, light brown, off-white, white or water-white.

A verifiable parameter to assess the color-removal efficiency of a decolorizing agent is the yellowness index (“YI”). The YI can be obtained from the Hunter color space L*, a*, and b* values obtained from colorimetric measurements of the reaction composition using a HunterLab color measurement instrument (Reston, Va.). The Hunter L*, a*, and b* values are then converted to International Commission on Illumination (“CIE”) xyz values for the 2 degree standard observer according to the following formulae:

$$\begin{matrix}y = {( \frac{L}{10} )}^{2} & ( Equation 1 ) \\ x = \frac{( \frac{a}{17.5} * \sqrt{y} ) + y}{1.02} & ( Equation 2 ) \\ z = \frac{( \frac{b}{7} * \sqrt{y} ) - y}{0.847} & ( Equation 3 )\end{matrix}$$

Once the CIE xyz values are determined, the following formula is used to calculate the YI:

$$Y I = \frac{100 * ( 1.28 x - 1.06 z )}{y} \qquad ( Equation 4 )$$

The YI-value can, for example, be determined analytically using a HUNTERLABULTRASCAN® VIS color measurement spectrophotometer. Typically, the YI is determined for a 4 mg/mL sample in a solvent, such as from about 0.01% to about 12% aqueous H₃PO₄ solution.

The YI can be used to assess efficiency of the decolorizing reaction during step (a). A higher YI-value represents a more intense color of the composition (comprising compounds of formulae (I) and (II)), resulting in a lower transparency of the product. A lower YI-value represents a reduced color intensity of the composition, resulting in a higher transparency of the product. For example, in a composition sample that has not been treated with a decolorizing agent, a high YI-value, e.g., of about 300, typically represents a substantially non-transparent sample, resulting in a dark-yellow- or brown-colored solution. In contrast, the same sample composition after decolorizing step (a) can encompass a lower YI-value, e.g., of about 20 or less, representing a substantially transparent sample exhibiting a light yellow, colorless or substantially colorless, e.g., water-white, solution.

In one embodiment, decolorizing step (a) is performed until the YI of the composition comprising compounds of formulae (I) and (II) or the salts or solvates thereof is less than about 100. In other embodiments, decolorizing step (a) is performed until the YI of the composition comprising compounds of formulae (I) and (II) or the salts or solvates thereof is less than about 50, less than about 25 or about 10 or less.

Typically, in decolorizing step (a), the decolorization of the composition comprising compounds of formulae (I) and (II) takes place in the presence of a decolorizing agent. A “decolorizing agent” of the disclosure encompasses all natural, synthetic and semi-synthetic substances which are able to partially or completely remove the color of the composition comprising compounds of formulae (I) and (II) or salts or solvates thereof. Without being bound by theory, it is believed that the decolorizing agent of optional decolorizing step (a) exerts its color-removing effect by physical absorption of colored particles present in the reaction composition.

Typically, the reaction conditions of decolorizing step (a) are adjusted to achieve the maximum removal of colorized particles from the reaction composition. In one embodiment, i.e., to achieve high removal rates, decolorizing step (a) is performed at elevated temperatures. In another embodiment, decolorizing step (a) is performed over a period from about 1 to about 16 hours.

In certain embodiments, the decolorizing agent is selected from the group consisting of carbon-based decolorizing agents, aluminum-based decolorizing agents, and mixtures thereof.

In certain embodiments, the aluminum-based decolorizing agent is Al₂O₃.

In a preferred embodiment, the decolorizing agent is characterized as activated carbon, such as Darco KB-G (Cabot Corp., Alpharetta, GA), or as activated charcoal from Sigma-Aldrich (St. Louis, Mo.), 100-400 mesh, which is believed to be identical to Darco KB-G.

The amount of decolorizing agent in decolorizing step (a) is selected such that said amount is sufficient to partially or completely remove the color of the reaction composition, i.e., such that the color of the reaction composition after decolorizing step (a) is yellow, white or water-white. The amount of decolorizing agent (e.g., in grams) added to the reaction composition comprising compounds of formulae (I) and (II) (e.g., in grams) can be defined as the weight of decolorizing agent per weight starting material comprising compounds of formulae (I) and (II) percentage (i.e., wt %).

In certain embodiments, the amount of decolorizing agent in decolorizing step (a) is from about 1 wt % to about 90 wt %, from about 10 wt % to about 80 wt %, from about 15 wt % to about 60 wt %, or from about 20 wt % to about 40 wt % based on the total weight of compounds of formulae (I) and (II). In a preferred embodiment, the amount of decolorizing agent is from about 10 wt % to about 80 wt %. In a more preferred embodiment, the amount of decolorizing agent is from about 15 wt % to about 60 wt %. In an even more preferred embodiment, the amount of decolorizing agent is from about 20 wt % to about 30 wt %. In a most preferred embodiment, the amount of decolorizing agent is about 25 wt %.

The contact time in decolorizing step (a) can be from about 1 minute to about 120 hours. In a preferred embodiment, the contact time in decolorizing step (a) is from about 1 hour to about 80 hours. In a more preferred embodiment, the reaction time in decolorizing step (a) is from about 2 hours to about 24 hours. In a most preferred embodiment, the reaction time in decolorizing step (a) is from about 4 hours to about 12 hours.

In a preferred embodiment, the temperature during decolorizing step (a) is from about 30° C. to about 105° C. In a more preferred embodiment, the temperature during decolorizing step (a) is from about 70° C. to about 105° C.

In decolorizing step (a), the composition comprising compounds of formulae (I) and (II) can be provided in a solution or in a suspension, each comprising the compounds of formulae (I) and (II), an acid and a solvent. A suitable solvent can be selected from the group consisting of water, alcohols (e.g., methanol, ethanol, propanol, iso-propanol, butanol, iso-butanol, tert-butanol, tert-amyl alcohol, etc.), aromatic hydrocarbons (e.g., benzene, toluene, xylene, etc.), which aromatic hydrocarbons are optionally halogenated (e.g., chlorobenzene, bromobenzene), aliphatic hydrocarbons (e.g., cyclohexane, cycloheptane), which aliphatic hydrocarbons are optionally substituted (e.g., chloroform, halothan), ethers (e.g., dioxane, tetrahydrofuran, diethylether, and the like), (C₁-C₄)alkyl esters of (C₁-C₄)alkanoic acids (e.g., methyl formate, methyl acetate, ethyl acetate, etc.), amides (e.g., DMF, diethylformamide, DMAc), other N—(C₁-C₄)alkyl substituted (C₁-C₄)alkanoic acid amides, NMP, formylmorpholine, and mixtures thereof.

In certain embodiments, in decolorizing step (a), the volume ratio of the solvent relative to the volume of the composition comprising compounds of formulae (I) and (II) is from about 1 volumes to about 20 volumes. In a preferred embodiment, the volume ratio of the solvent relative to the volume of the composition comprising compounds of formulae (I) and (II) is from about 1 volume to about 12 volumes. In a more preferred embodiment, the volume ratio of the solvent is from about 2 volumes to about 8 volumes. In a more preferred embodiment, the volume ratio of the solvent is from about 2 volumes to about 4 volumes.

In certain embodiments, the acid H⁺ₙXⁿ⁻ can be added to the reaction composition of decolorizing step (a) as acid H⁺ₙXⁿ⁻ or can be generated in situ in the reaction composition from a salt containing an anion Xⁿ⁻.

The acid H⁺ₙXⁿ⁻ can be added (or generated in situ) before, during or before and during decolorizing step (a). The acid can be added once, in several portions or continuously over a certain period of time. It can be added once or at a plurality of times with respect to the decolorization reaction, e.g., before or during the decolorization reaction. If the acid is added (or generated in situ) before, during, or before and during the decolorization reaction, the process comprising decolorizing step (a) can be performed as a one-pot-reaction. Especially preferred is a process where the acid H⁺ₙXⁿ⁻ is added to (or generated in situ) the reaction mixture before decolorizing step (a).

The acid H⁺ₙXⁿ⁻ can be any acid containing an anion Xⁿ⁻ as defined herein. The acid can, for example, be HCl, H₂SO₄, the mono-salt of H₂SO₄, methanesulfonic acid, tosylic acid, trifluoroacetic acid, H₃PO₄, a mono-salt of H₃PO₄, a bi-salt of H₃PO₄, oxalic acid, perchloric acid, HC(O)OH, CH₃C(O)OH, or mixtures thereof.

In a preferred embodiment, the acid H⁺ₙXⁿ⁻ can be H₂SO₄, H₃PO₄, HC(O)OH, CH₃C(O)OH, or mixtures thereof. In a more preferred embodiment, the acid H⁺ₙXⁿ⁻ is H₃PO₄.

The ratio of the amount of acid H⁺ₙXⁿ⁻ to the composition comprising compounds of formulae (I) and (II) is selected such that the composition comprising compounds of formulae (I) and (II) is completely dissolved in the acid-solvent admixture, i.e., such that a solution of the composition comprising compounds of formulae (I) and (II) is formed.

In a preferred embodiment, the amount of acid is at least about 0.5 and preferably from about 0.5 to about 10 molar equivalents based on the total molar equivalent of compounds of formulae (I) and (II) or (Ia) and (IIa). In a more preferred embodiment, the amount of acid is from about 1 to about 6 molar equivalents. In an even more preferred embodiment, the amount of acid is from about 2 to about 3 molar equivalents. In a most preferred embodiment, the amount of acid is from about 2.2 to about 2.6 molar equivalents.

In certain embodiments, the amount of H⁺ provided by H⁺ₙXⁿ⁻ in decolorizing step (a) is in a slight molar excess in comparison to the composition comprising compounds of formulae (I) and (II). In certain embodiments, the molar amount of H⁺ₙXⁿ⁻ present in decolorizing step (a) is within a range of from about 1.1(1/n) to about 1.2(1/n) molar equivalents per molar equivalent of the composition comprising compounds of formulae (I) and (II).

In certain embodiments, the acid H⁺ₙXⁿ⁻ is the only acid used during decolorizing step (a). In another embodiment, one or more additional acids are added to the reaction mixture in addition to the acid H⁺ₙXⁿ⁻. Said additional acid or acids can be any acids selected from the group of acids as defined for the acid H⁺ₙXⁿ⁻ and mixtures of said acids.

In a preferred embodiment, the acid H⁺ₙXⁿ is H₃PO₄ and the amount of acid is at least about 0.5 equivalents based on the total molar equivalent of compounds of formulae (I) and (II) or (Ia) and (IIa). Preferably, the amount of acid is from about 0.5 to about 10 molar equivalents based on the total molar equivalent of compounds of formulae (I) and (II) or (Ia) and (IIa). In a more preferred embodiment, the amount of H₃PO₄ is from about 1 to about 6 molar equivalents. In an even more preferred embodiment, the amount of H₃PO₄ is from about 2 to about 3 molar equivalents. In a most preferred embodiment, the amount of H₃PO₄ is from about 2.2 to about 2.6 molar equivalents.

In a preferred embodiment, the process of the disclosure reduces 14-hydroxynormorphinone (as the compound of formula (I)) or a salt or a solvate thereof in a composition comprising 14-hydroxynormorphinone and noroxymorphone (as the compound of formula (II)) or salts or solvates thereof in the presence of H₃PO₄, and the amount of H₃PO₄ is from about 0.5 to about 10 molar equivalents based on the total molar equivalent of compounds of formulae (I) and (II). In a more preferred aspect of this embodiment, the amount of H₃PO₄ is from about 1 to about 6 molar equivalents. In an even more preferred aspect of this embodiment, the amount of H₃PO₄ is from about 2 to about 3 molar equivalents. In a most preferred aspect of this embodiment, the amount of H₃PO₄ is from about 2.2 to about 2.6 molar equivalents.

After completion of decolorizing step (a), the decolorizing agent can be removed by any suitable means such as by filtration. The filtrate obtained after filtration may be passed through the same filter a plurality of times to maximize the removal efficiency of the retained filter cake. In one embodiment, the filter cake is not washed.

In one embodiment, to increase filtering efficiency by minimizing the loss of the composition comprising compounds of formulae (I) and (II) during filtration, the filtration step includes from about 1 to about 20 washing steps. In a preferred embodiment, the filtration step includes from about 1 to about 10 washing steps. In a more preferred embodiment, the filtration step includes from about 1 to about 5 washing steps. In an even more preferred embodiment, the filtration step includes from about 2 to about 4 washing steps. In a most preferred embodiment, the filtration step includes about 3 washing steps.

Typically, to increase the filtering efficiency the filtration temperature of the composition can be controlled during decolorizing step (a). In one embodiment, the filtration temperature is from about 0° C. to about 100° C. In a preferred embodiment, the filtration temperature is from about 15° C. to about 100° C. In a more preferred embodiment, the filtration temperature is from about 20° C. to about 90° C. In a most preferred embodiment, the filtration temperature is from about 50° C. to about 70° C.

In certain embodiments, the filtered decolorizing agent can be washed with a wash solvent. In one embodiment, the washing solvent is selected from the group consisting of water, alcohol, and mixtures thereof. In a preferred embodiment, the washing solvent is selected from the group consisting of water, methanol, ethanol, propanol, iso-propanol, butanol, iso-butanol, tert-butanol, tert-amyl alcohol, and mixtures thereof. In a more preferred embodiment, the wash solvent is water.

In certain embodiments, the amount of washing solvent used for washing the filtered decolorizing agent is from about 1 to about 10 volumes based on the total mass of the filtered decolorizing agent, i.e., the filter cake. In a preferred embodiment, the amount of washing solvent used is from about 1 to about 5 volumes. In a more preferred embodiment, the amount of washing solvent used is about 2 volumes based on the total mass of the filtered decolorizing agent.

The decolorization reaction can take place in any suitable reaction vessel. In certain embodiments, the reaction vessel is a flow reactor. In certain embodiments, the reaction vessel is a continuous flow reactor. In certain other embodiments, the reaction vessel is not a continuous flow reactor. In certain other embodiments, the reaction vessel is not a flow reactor. Non-limiting examples of the material or materials making up a suitable reaction vessel include grade 316 stainless steel, a HASTELLOY corrosion-resistant metal alloy, glass, or a glass lining.

### C. Preferred Embodiments of the Process

In a preferred embodiment, the compound of formula (I) is 14-hydroxynormorphinone or a salt or a solvate thereof, H₃PO₄ is added to the reaction composition, and the compound of formula (II) is noroxymorphone or a salt or a solvate thereof. The hydrogenation catalyst used in hydrogenating step (b) is palladium on carbon (e.g., 5 wt %). The amount of 5 wt % palladium on carbon can be about 1.8 wt % based on the total weight of compounds of formulae (I) and (II). Further, the amount of H₃PO₄ added to the reaction mixture is from about 2.2 to about 2.6 molar equivalents based on the total molar equivalent of compounds of formulae (I) and (II). The hydrogenation process is optionally performed in the presence of a halide-containing compound, which is preferably selected as sodium iodide or sodium chloride. The halide-containing compound is present from about 0.0001 wt % to about 1 wt % and is preferably present from about 0.0025 wt % to about 0.1 wt % based on the total weight of compounds of formulae (I) and (II). After hydrogenation, a base, such as ammonium hydroxide, is added in salt-breaking step (c).

In a preferred embodiment, during base addition the temperature of the product of hydrogenating step (b) when a first portion of the base in salt-breaking step (c) is added thereto is from about 20° C. to about 30° C., the first portion of the base is added until the pH is adjusted to from about 4.5 to about 5.5, and the temperature when a second portion of the base in salt-breaking step (c) is added thereto is from about 70° C. to about 80° C. until a pH of from about 7.5 to about 8.5 is reached.

In a preferred embodiment, decolorizing step (a) is performed before hydrogenating step (b).

In a preferred embodiment, activated carbon is used as decolorizing agent in decolorizing step (a). In a preferred embodiment, the amount of activated carbon is about 25 wt % based on the total weight of compounds of formulae (I) and (II).

In a preferred embodiment, the temperature during decolorizing step (a) is about 90° C.

In a preferred embodiment, the duration of decolorizing step (a) is about 6 hours.

### D. ABUK Levels Present in the Composition Comprising Compounds of Formulae (I) and (II)

The process of the disclosure comprises the reduction of the amount of a compound of formula (I) or a salt or a solvate thereof in a composition comprising compounds of formulae (I) and (II) or salts or solvates thereof, where the process comprises hydrogenating the compound of formula (I). Furthermore, the process of the disclosure comprises the optional decolorizing step (a), the optional salt-breaking step (c), or the optional decolorizing step (a) and the optional salt-breaking step (c).

An objective of the process of the disclosure is to obtain a product composition, where the amount of the compound of formula (I) or a salt or a solvate thereof has been reduced relative to the amount of the compound of formula (I) or a salt or a solvate thereof present in the reaction composition before hydrogenating step (b).

In certain embodiments, the process of the disclosure results in a product composition where the amount of the compound of formula (I) or a salt or a solvate thereof relative to the amount of the compound of formula (II) or a salt or a solvate thereof in the product is less than about 200 ppm, less than about 150 ppm, less than about 100 ppm, less than about 90 ppm, less than about 75 ppm, less than about 50 ppm, less than about 40 ppm, less than about 35 ppm, less than about 25 ppm, less than about 10 ppm or less than 5 ppm.

In certain embodiments, the process of the disclosure results in a product composition where the amount of the compound of formula (I) or a salt or a solvate thereof relative to the amount of the compound of formula (II) or a salt or a solvate thereof in the product is from about 5 ppm to less than about 200 ppm, preferably from about 10 ppm to less than about 200 ppm. In a preferred embodiment, the amount of the compound of formula (I) or a salt or a solvate thereof relative to the amount of the compound of formula (II) or a salt or a solvate thereof in the product is from about 5 ppm to less than about 150 ppm, preferably from about 10 ppm to less than about 150 ppm. In a more preferred embodiment, the amount of the compound of formula (I) or a salt or a solvate thereof relative to the amount of the compound of formula (II) or a salt or a solvate thereof in the product is from about 5 ppm to less than about 100 ppm, preferably from about 10 ppm to less than about 100 ppm. In an even more preferred embodiment, the amount of the compound of formula (I) or a salt or a solvate thereof relative to the amount of the compound of formula (II) or a salt or a solvate thereof in the product is from about 5 ppm to less than about 75 ppm, preferably from about 10 ppm to less than about 75 ppm. In a further preferred embodiment, the amount of the compound of formula (I) or a salt or a solvate thereof relative to the amount of the compound of formula (II) or a salt or a solvate thereof in the product is from about 5 ppm to less than about 50 ppm, preferably from about 10 ppm to less than about 50 ppm. In an even further preferred embodiment, the amount of the compound of formula (I) or a salt or a solvate thereof relative to the amount of the compound of formula (II) or a salt or a solvate thereof in the product is from about 5 ppm to less than about 40 ppm, preferably from about 10 ppm to less than about 40 ppm. In an even further preferred embodiment, the amount of the compound of formula (I) or a salt or a solvate thereof relative to the amount of the compound of formula (II) or a salt or a solvate thereof in the product is from about 5 ppm to less than about 35 ppm, preferably from about 10 ppm to less than about 35 ppm. In a most preferred embodiment, the amount of the compound of formula (I) or a salt or a solvate thereof relative to the amount of the compound of formula (II) or a salt or a solvate thereof in the product is from about 5 ppm to less than about 25 ppm, preferably from about 10 ppm to less than about 25 ppm.

In certain embodiments, the compound of formula (I) is 14-hydroxynormorphinone or a salt or a solvate thereof, and the compound of formula (II) is noroxymorphone or a salt or a solvate thereof, where the amount of 14-hydroxynormorphinone or a salt or a solvate thereof relative to the amount of noroxymorphone or a salt or a solvate thereof in the product is less than about 200 ppm, less than about 150 ppm, less than about 100 ppm, less than about 75 ppm, less than about 50 ppm, less than about 40 ppm, less than about 35 ppm, less than about 25 ppm, less than about 10 ppm or less than 5 ppm.

In certain embodiments, the compound of formula (I) is 14-hydroxynormorphinone or a salt or a solvate thereof, and the compound of formula (II) is noroxymorphone or a salt or a solvate thereof, where the amount of 14-hydroxynormorphinone or a salt or a solvate thereof relative to the amount of noroxymorphone or a salt or a solvate thereof in the product is from about 5 ppm to less than about 200 ppm, from about 10 ppm to less than about 200 ppm, from about 5 ppm to less than about 150 ppm, from about 10 ppm to less than about 150 ppm, from about 5 ppm to less than about 100 ppm, from about 10 ppm to less than about 100 ppm, from about 5 ppm to less than about 75 ppm, from about 10 ppm to less than about 75 ppm, from about 5 ppm to less than about 50 ppm, from about 10 ppm to less than about 50 ppm, from about 5 ppm to less than about 40 ppm, from about 10 ppm to less than about 40 ppm, from about 5 ppm to less than about 35 ppm, from about 10 ppm to less than about 35 ppm, from about 5 ppm to less than about 25 ppm or from about 10 ppm to less than about 25 ppm.

In certain embodiments, the compound of formula (I) is 7,8-didehydronaloxone or a salt or a solvate thereof, and the compound of formula (II) is naloxone or a salt or a solvate thereof, where the amount of 7,8-didehydronaloxone or a salt or a solvate thereof relative to the amount of naloxone or a salt or a solvate thereof in the product is less than about 200 ppm, less than about 150 ppm, less than about 100 ppm, less than about 75 ppm, less than about 50 ppm, less than about 40 ppm, less than about 35 ppm, less than about 25 ppm, less than about 10 ppm or less than 5 ppm.

In certain embodiments, the compound of formula (I) is 7,8-didehydronaloxone or a salt or a solvate thereof, and the compound of formula (II) is naloxone or a salt or a solvate thereof, where the amount of 7,8-didehydronaloxone or a salt or a solvate thereof relative to the amount of naloxone or a salt or a solvate thereof in the product is from about 5 ppm to less than about 200 ppm, from about 10 ppm to less than about 200 ppm, from about 5 ppm to less than about 150 ppm, from about 10 ppm to less than about 150 ppm, from about 5 ppm to less than about 100 ppm, from about 10 ppm to less than about 100 ppm, from about 5 ppm to less than about 75 ppm, from about 10 ppm to less than about 75 ppm, from about 5 ppm to less than about 50 ppm, from about 10 ppm to less than about 50 ppm, from about 5 ppm to less than about 40 ppm, from about 10 ppm to less than about 40 ppm, from about 5 ppm to less than about 35 ppm, from about 10 ppm to less than about 35 ppm, from about 5 ppm to less than about 25 ppm or from about 10 ppm to less than about 25 ppm.

In certain embodiments, the compound of formula (I) is 7,8-didehydronaltrexone or a salt or a solvate thereof, and the compound of formula (II) is naltrexone or a salt or a solvate thereof, where the amount of 7,8-didehydronaltrexone or a salt or a solvate thereof relative to the amount of naltrexone or a salt or a solvate thereof in the product is less than about 200 ppm, less than about 150 ppm, less than about 100 ppm, less than about 75 ppm, less than about 50 ppm, less than about 40 ppm, less than about 35 ppm, less than about 25 ppm, less than about 10 ppm or less than 5 ppm.

In certain embodiments, the compound of formula (I) is 7,8-didehydronaltrexone or a salt or a solvate thereof, and the compound of formula (II) is naltrexone or a salt or a solvate thereof, where the amount of 7,8-didehydronaltrexone or a salt or a solvate thereof relative to the amount of naltrexone or a salt or a solvate thereof in the product is from about 5 ppm to less than about 200 ppm, from about 10 ppm to less than about 200 ppm, from about 5 ppm to less than about 150 ppm, from about 10 ppm to less than about 150 ppm, from about 5 ppm to less than about 100 ppm, from about 10 ppm to less than about 100 ppm, from about 5 ppm to less than about 75 ppm, from about 10 ppm to less than about 75 ppm, from about 5 ppm to less than about 50 ppm, from about 10 ppm to less than about 50 ppm, from about 5 ppm to less than about 40 ppm, from about 10 ppm to less than about 40 ppm, from about 5 ppm to less than about 35 ppm, from about 10 ppm to less than about 35 ppm, from about 5 ppm to less than about 25 ppm or from about 10 ppm to less than about 25 ppm.

Further, the process of the disclosure optionally comprises a decolorizing step (a), where the color of the product composition is reduced as measured by the YI, which is defined above.

In certain embodiments, the YI of the composition comprising compounds of formulae (I) and (II) or the salts or solvates thereof in the product is less than about 100, less than about 50, less than about 25 or less than about 10.

In preferred embodiments, the process of the disclosure results in a product composition where the amount of the compound of formula (I) or a salt or a solvate thereof relative to the amount of the compound of formula (II) or a salt or a solvate thereof in the product is less than about 200 ppm, less than about 100 ppm, preferably less than about 75 ppm, and more preferably less than about 50 ppm, even more preferably less than about 10 ppm and most preferably less than about 5 ppm, and where the amount of a compound of formula (IV) or a salt or a solvate thereof is less than 0.5 HPLC peak area ratio, preferably less than 0.25 HPLC peak area ratio, and more preferably less than 0.15 HPLC peak area ratio. The HPLC peak area ratio refers to the area under the HPLC peak corresponding to the compound of formula (IV) divided by the area under the HPLC peak corresponding to the compound of formula (II), each HPLC peak being preferably determined according to the procedure provided in Example 1.3.

In more preferred embodiments, the process of the disclosure results in a product composition where the compound of formula (I) is 14-hydroxynormorphinone, the compound of formula (II) is noroxymorphone and the compound of formula (IV) is 3,4,14-trihydroxymorphinan-6-one, where the amount of 14-hydroxynormorphinone or a salt or a solvate thereof relative to the amount of noroxymorphone or a salt or a solvate thereof in the product is less than about 200 ppm, less than about 100 ppm, preferably less than about 75 ppm, more preferably less than about 50 ppm, even more preferably less than about 10 ppm and most preferably less than 5 ppm, and where the amount of 3,4,14-trihydroxymorphinan-6-one or a salt or a solvate thereof is less than 0.5 HPLC peak area ratio, preferably less than 0.25 HPLC peak area ratio, and more preferably less than 0.15 HPLC peak area ratio. The method for determining the HPLC peak area ratio of 3,4,14-trihydroxymorphinan-6-one can be the same as shown in Example 1.2. herein below. The HPLC peak area ratio refers to the area under the peak corresponding to 3,4,14-trihydroxymorphinan-6-one divided by the area under the peak corresponding to noroxymorphone. The method for determining the ppm amount of 14-hydroxynormorphinone can be the same as shown in the HPLC Example 1.1. herein below.

### E. Levels of the Compound of Formula (III) in the Composition Comprising Compounds of Formulae (I) and (II)

The disclosure provides a process for reducing the amount of a compound of formula (I) or a salt or a solvate thereof, in a composition comprising compounds of formulae (I) and (II) or salts or solvates thereof, the process comprising hydrogenating the compound of formula (I).

During Stage 1 of naloxone synthesis (see Scheme 1) or naltrexone synthesis (see Schemes 1 and 2), which includes oxidation and reduction steps, certain by-products can be formed which can be present in the initial noroxymorphone composition. These reaction by-products can also be formed during any or all of the reaction steps of decolorizing step (a), hydrogenating step (b), and salt-breaking step (c).

The product of the process of the disclosure may contain a composition, which further comprises a compound of formula (III) as a by-product:

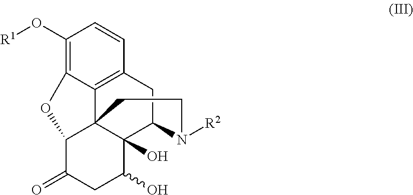

or a salt or a solvate thereof; where:
R¹ is —H, (C₁-C₇)alkyl, or an O-protecting group; and
R² is —H, —(C₂-C₄)alkenyl, —(C₂-C₇)alkyl, —(C₁-C₇)alkyl-(C₃-C₇)cycloalkyl, —CN, —C(═O)O—(C₁-C₆)alkyl, —C(═O)O-phenyl or an N-protecting group.

In a preferred embodiment, R¹ is —H.

In a preferred embodiment, R² is —H.

In a preferred embodiment, R² is —(C₂-C₄)alkenyl or —(C₁-C₇)alkyl-(C₃-C₇)cycloalkyl.

In a preferred embodiment, R² is —CH₂CH═CH₂.

In another preferred embodiment, R² is —CH₂-cyclopropyl.

In a certain embodiment, the compound of formula (III) is:

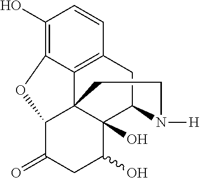 [SMILES: O=C1CC(O)[C@@]2(O)[C@H]3Cc4ccc(O)c5c4[C@@]2(CCN3)[C@H]1O5]

or a salt or a solvate thereof.

In a certain embodiment, the compound of formula (III) is:

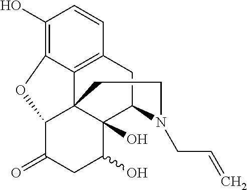 [SMILES: C=CCN1CC[C@]23c4c5ccc(O)c4O[C@H]2C(=O)CC(O)[C@@]3(O)[C@H]1C5]

or a salt or a solvate thereof.

In a certain embodiment, the compound of formula (III) is:

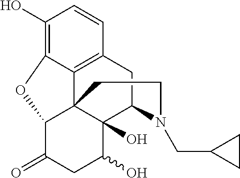 [SMILES: O=C1CC(O)[C@@]2(O)[C@H]3Cc4ccc(O)c5c4[C@@]2(CCN3CC2CC2)[C@H]1O5]

or a salt or a solvate thereof.

Said compound of formula (III) can be present in the product in the form of its free base, or in the form of its salt of formula (IIIa):

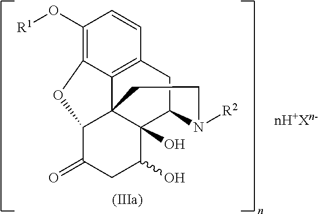

or a solvate thereof; where:
R¹ is —H, (C₁-C₇)alkyl, or an O-protecting group;
R² is —H, —(C₂-C₄)alkenyl, —(C₂-C₇)alkyl, —(C₁-C₇)alkyl-(C₃-C₇)cycloalkyl, —CN, —C(=O)O —(C₁-C₆)alkyl, —C(═O)O-phenyl or an N-protecting group;
Xⁿ⁻ is an anion selected from the group consisting of I⁻, F⁻, valerate, acetate, meconate, salicylate, barbiturate, Br⁻, succinate, tartrate, maleate, fumarate, citrate, NO₃⁻, Cl⁻, HSO₄⁻, SO₄²⁻, methanesulfonate, tosylate, trifluoroacetate, H₂PO₄⁻, HPO₄²⁻, PO₄³⁻, [(NH₄)HPO₄]⁻, [(NH₄)₂PO₄]⁻, oxalate, perchlorate, H₃CC(O)O⁻, HC(O)O⁻, and mixtures thereof; and
n is 1, 2 or 3.

In another embodiment, for the compound of formula (IIIa):
Xⁿ⁻ is an anion selected from the group consisting of I⁻, F⁻, valerate, acetate, meconate, salicylate, barbiturate, Br⁻, succinate, tartrate, maleate, fumarate, citrate, NO₃⁻, Cl⁻, HSO₄⁻, SO₄²⁻, methanesulfonate, tosylate, trifluoroacetate, H₂PO₄⁻, HPO₄²⁻, [(NH₄)HPO₄]⁻, oxalate, perchlorate, H₃CC(O)O⁻, HC(O)O⁻, and mixtures thereof; and
n is 1 or 2.

Under the acidic conditions of the hydrogenation reaction of the disclosure, the compound of formula (III) is typically present in its protonated form and will therefore form a salt or a solvate of formula (IIIa).

Said compound of formula (III) can be present in the composition comprising compounds of formulae (I) and (II) at the end of the process in dissolved or precipitated form. In embodiments where the composition comprising compounds of formulae (I) and (II) is precipitated, said compound of formula (III) can be present in the precipitate, in the mother liquor, or in both.

Whenever a compound of formula (III) is present in the process product, it is present in a certain amount which shall be specified in the following.

In certain embodiments, the amount of the compound of formula (III) or a salt or a solvate thereof which is present in the process product is more than the amount of the compound of formula (III) or a salt or a solvate thereof which was present before undergoing the process of the disclosure.

In certain embodiments, the amount of the compound of formula (III) or a salt or a solvate thereof which is present in the process product is less than the amount of the compound of formula (III) or a salt or a solvate thereof which was present before undergoing the process of the disclosure.

In certain embodiments, the composition comprising compounds of formulae (I) and (II) or solvates thereof is precipitated during the process and the precipitate contains less compound of formula (III) or a salt or a solvate thereof relative to the composition comprising compounds of formulae (I) and (II) or a solvate thereof than the mother liquor.

In certain embodiments, the process product composition comprising compound of formulae (I) and (II) contains the 8α-epimer, the 8β-epimer, or a mixture of these two epimers of a compound of formula (III). Preferably, the composition contains the 8β-epimer. Without being bound by theory, it is believed that the 8β-epimer is less likely to be dehydrated into a compound of formula (I), e.g., due to the acidic reaction conditions.

In preferred embodiments, the amount of 8α-stereoisomer, which is present in the process product, is less than the amount of 8β-stereoisomer.

In one embodiment, the compound of formula (I) is 14-hydroxynormorphinone or a salt or a solvate thereof, e.g., 14-hydroxynormorphinone hydrogen phosphate, and the compound of formula (III) or a salt or a solvate thereof is 8-hydroxynoroxymorphone having 8α-, 8β-, or 8α- and 8β-stereo-configuration. In one embodiment, the 8-hydroxynoroxymorphone is predominantly (i.e., greater than 50%) in the 8β-stereo-configuration.

In one embodiment, the compound of formula (I) is 7,8-didehydronaloxone or a salt or a solvate thereof, e.g., 7,8-didehydronaloxone hydrogen phosphate, and the compound of formula (III) or a salt or a solvate thereof is 8-hydroxynaloxone having 8α-, 8β-, or 8α- and 8β-stereo-configuration. In one embodiment, the 8-hydroxynaloxone is predominantly (i.e., greater than 50%) in the 8β-stereo-configuration.

In one embodiment, the compound of formula (I) is 7,8-didehydronaltrexone or a salt or a solvate thereof, e.g., 7,8-didehydronaltrexone hydrogen phosphate, and the compound of formula (III) or a salt or a solvate thereof is 8-hydroxynaltrexone having 8α-, 8β-, or 8α- and 8β-stereo-configuration. In one embodiment, the 8-hydroxynaltrexone is predominantly (i.e., greater than 50%) in the 8β-stereo-configuration.

### F. Further Processing of the Composition Comprising Compounds of Formulae (I) and (II) or the Salts or Solvates Thereof

In certain embodiments, the isolated process products comprising the composition comprising compounds of formulae (I) and (II) or the salts or solvates thereof can be further processed.

In certain embodiments, the isolated process product comprising the composition comprising compounds of formulae (I), (II) and (III) or solvates thereof can be washed with, crystallized or recrystallized in, or washed with and crystallized or recrystallized in an organic solvent, an aqueous solvent, or mixtures thereof, in which a compound of formula (III) or a salt or a solvate thereof is more soluble than the compounds of formulae (I) and (II) or salts or solvates thereof.

In certain embodiments, the isolated process product comprising the composition comprising compounds of formulae (I), (II) and optionally compounds of formula (III) can be washed with, crystallized or recrystallized in, or washed with and crystallized or recrystallized in an organic solvent, an aqueous solvent, or mixtures thereof, in which a compound of formula (I) or a salt or a solvate thereof is more soluble than compound of formula (II) in the composition or a salt or a solvate thereof.

The washing, crystallization or recrystallization, or washing and crystallization or recrystallization can further reduce the amount of the compound of formula (I), the amount of the compound of formula (III), or the amount of the compounds of formulae (I) and (III) in the isolated precipitate containing the composition comprising compounds of formulae (I) and (II) or a solvate thereof. The washing, crystallization or recrystallization, or washing and crystallization or recrystallization can be performed more than once, or they can be carried out sequentially.

In certain embodiments, the isolated process product comprises a composition where the compound of formula (I) is 14-hydroxynormorphinone and the compound of formula (II) is noroxymorphone and said composition is used as a starting material for the synthesis of naloxone, naltrexone or salts or solvates thereof.

In certain embodiments, the process for preparing naloxone or a pharmaceutically acceptable salt or solvate thereof, comprises the steps of:

i) providing a composition comprising compounds of formulae (I) and (II) or pharmaceutically acceptable salts or solvates thereof, where:
R¹ is —H, (C₁-C₇)alkyl, or an O-protecting group; and
R² is —H, —(C₂-C₄)alkenyl, —(C₂-C₇)alkyl, —(C₁-C₇)alkyl-(C₃-C₇)cycloalkyl, —CN, —C(═O)O—(C₁-C₆)alkyl, —C(═O)O-phenyl or an N-protecting group;
and where the amount of the compound of formula (I) in the composition relative to the amount of the compound of formula (II) is less than about 200 ppm, less than about 150 ppm, less than about 100 ppm, less than about 50 ppm, less than about 40 ppm, less than about 35 ppm, less than about 25 ppm, less than about 10 ppm or less than about 5 ppm; and where preferably R² is selected as —H; and

ii) reacting the composition of i) with an alkylating agent to form naloxone or a pharmaceutically acceptable salt or solvate thereof, where the amount of 7,8-didehydronaloxone in the composition relative to the amount of naloxone is less than about 200 ppm, less than about 150 ppm, less than about 100 ppm, less than about 50 ppm, less than about 40 ppm, less than about 35 ppm, less than about 25 ppm, less than about 10 ppm or less than 5 ppm.

In one preferred embodiment, the process product is used for the synthesis of naloxone or a salt of solvate thereof. In a preferred embodiment, the synthesis of naloxone comprises the steps of alkylation by the use of an alkylating agent (Stage 3) as described in Scheme 1. In a preferred embodiment, the alkylating agent is an allyl halide. Preferably, the allyl halide is allyl bromide. In yet another embodiment, the synthesis of naloxone hydrochloride comprises the step of salt formation (Stage 4) as described in Scheme 1.

In certain embodiments, the process for preparing naltrexone or a pharmaceutically acceptable salt or solvate thereof, comprises the steps of:

i) providing a composition comprising compounds of formulae (I) and (II) or pharmaceutically acceptable salts or solvates thereof, where:
R¹ is —H, (C₁-C₇)alkyl, or an O-protecting group; and
R² is —H, —(C₂-C₄)alkenyl, —(C₂-C₇)alkyl, —(C₁-C₇)alkyl-(C₃-C₇)cycloalkyl, —CN, —C(═O)O—(C₁-C₆)alkyl, —C(═O)O-phenyl or an N-protecting group;
and where the amount of the compound of formula (I) in the composition relative to the amount of the compound of formula (II) is less than about 200 ppm, less than about 150 ppm, less than about 100 ppm, less than about 50 ppm, less than about 40 ppm, less than about 35 ppm, less than about 25 ppm, less than about 10 ppm or less than about 5 ppm; and where preferably R² is selected as —H; and

ii) reacting the composition of i) with an alkylating agent to form naltrexone or a pharmaceutically acceptable salt or solvate thereof, where the amount of 7,8-didehydronaltrexone in the composition relative to the amount of naltrexone is less than about 200 ppm, less than about 150 ppm, less than about 100 ppm, less than about 40 ppm, less than about 50 ppm, less than about 35 ppm, less than about 25 ppm, less than about 10 ppm or less than 5 ppm.

In one preferred embodiment, the process product is used for the synthesis of naltrexone or a salt of solvate thereof. In a preferred embodiment, the synthesis of naltrexone comprises the steps of alkylation by the use of an alkylating agent (Stage 3) as described in Scheme 2. In a preferred embodiment, the alkylating agent is a cyclopropylmethyl halide. Preferably, the cyclopropylmethyl halide is cyclopropylmethyl bromide. In yet another embodiment, the synthesis of naltrexone hydrochloride comprises the step of salt formation (Stage 4) as described in Scheme 2.

In another preferred embodiment, the synthesis of naloxone, naltrexone or salts or solvates thereof starting from the process product of the disclosure comprising of the composition of noroxymorphone and 14-hydroxynormorphinone or salts or solvates thereof consists of reaction steps where the amount of ABUK in the final product is less than 90 ppm in one embodiment and less than 75 ppm in another embodiment.

In yet another preferred embodiment, the process product comprises a composition where the compound of formula (I) is 14-hydroxynormorphinone, the compound of formula (II) is noroxymorphone and said composition is used as a starting material for the synthesis of a composition comprising naloxone, naloxone hydrochloride and the ABUK 7,8-didehydronaloxone or a salt or a solvate thereof, where the amount of 7,8-didehydronaloxone is less than 100 ppm, preferably less than 75 ppm, more preferably less than 50 ppm, even more preferably less than 25 ppm and most preferably less than 10 ppm relative to the amount of naloxone or naloxone hydrochloride. The amount of 7,8-didehydronaloxone can be determined by HPLC, for example, as described in Example 1.3. herein below.

In yet another preferred embodiment, the process product comprises a composition where the compound of formula (I) is 14-hydroxynormorphinone, the compound of formula (II) is noroxymorphone and said composition is used as a starting material for the synthesis of a composition comprising naltrexone, naltrexone hydrochloride and the ABUK 7,8-didehydronaltrexone or a salt or a solvate thereof, where the amount of 7,8-didehydronaltrexone is less than 100 ppm, preferably less than 75 ppm, more preferably less than 50 ppm, even more preferably less than 25 ppm and most preferably less than 10 ppm relative to the amount of naltrexone or naltrexone hydrochloride. The amount of 7,8-didehydronaltrexone can be determined by HPLC, for example, as described in Example 1.4. herein below.

In an alternative preferred embodiment, the process product comprises a compound of formula (I) or a salt or a solvate thereof, a compound of formula (II) or a salt or a solvate thereof, the isolated process product composition contains less than 100 ppm, preferably less than 75 ppm, more preferably less than 50 ppm, even more preferably less than 25 ppm, and most preferably less than 10 ppm of the compound of formula (I) or a salt or a solvate thereof relative to the compound of formula (II) or a salt or a solvate thereof, and the composition is used in a reaction with an alkylating agent, such as the non-limiting alkylating agent 2-propenyl halide illustrated in the scheme, to give compounds of formula (Ie) and formula (IIe) as shown in Scheme 11 below.

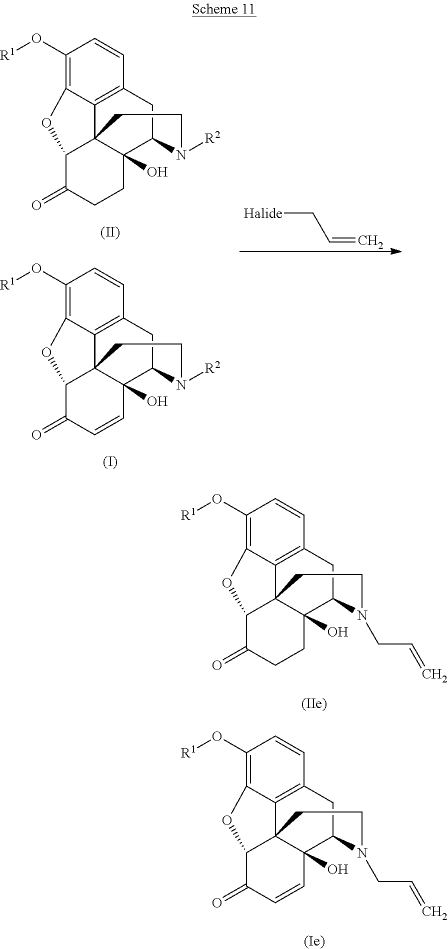

where:
R¹ is —H, (C₁-C₇)alkyl, or an O-protecting group;
R² is H; and
halide is Cl, Br or I.

In a preferred embodiment, the 2-propenyl halide is Cl—CH₂CH═CH₂, Br—CH₂CH═CH₂, I—CH₂CH═CH₂ and preferably is Cl—CH₂CH═CH₂. In an more preferred embodiment, the compound of formula (Ie) is less than about 100 ppm, preferably less than about 75 ppm, more preferably less than about 50 ppm, even more preferably less than about 25 ppm and most preferably less than about 10 ppm relative to the compound of formula (lie) or a salt or a solvate thereof. In a preferred embodiment, R₁ is H.

In another alternative preferred embodiment, the process product comprises a compound of formula (I) or a salt or a solvate thereof, a compound of formula (II) or a salt or a solvate thereof, the isolated process product composition contains less than 100 ppm, preferably less than 75 ppm, more preferably less than 50 ppm, even more preferably less than 25 ppm, and most preferably less than 10 ppm of compound of formula (I) or a salt or a solvate thereof relative to the compound of formula (II) or a salt or a solvate thereof, and the composition is used in a reaction with an alkylating agent, such as the non-limiting alkylating agent cyclopropylmethyl halide illustrated in the scheme, to give compounds of formula (If) and formula (IID as shown in Scheme 12 below.

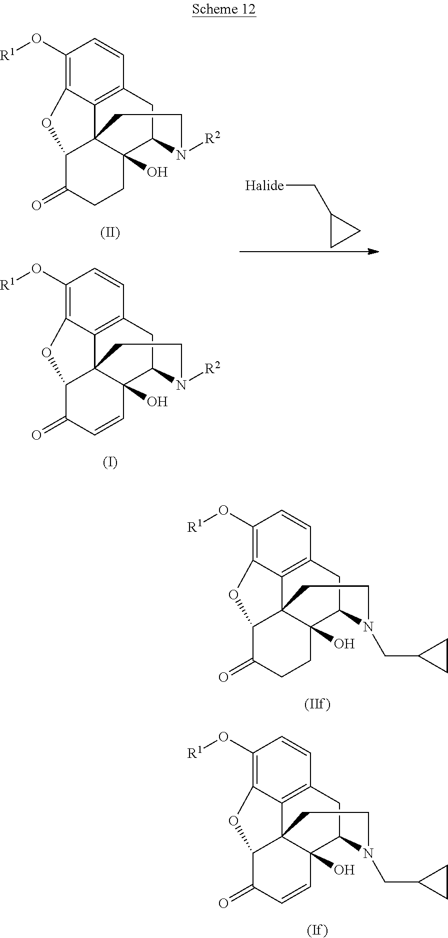

where:
R¹ is —H, (C₁-C₇)alkyl, or an O-protecting group;
R² is H; and
halide is Cl, Br or I.

In a preferred embodiment, the cyclopropylmethyl halide is cyclopropylmethyl chloride, cyclopropylmethyl bromide or cyclopropylmethyl iodide and preferably is cyclopropylmethyl bromide. In an more preferred embodiment, the compound of formula (If) is less than about 100 ppm, preferably less than about 75 ppm, more preferably less than about 50 ppm, even more preferably less than about 25 ppm and most preferably less than about 10 ppm relative to the compound of formula (IID or a salt or a solvate thereof. In a preferred embodiment, R₁ is H.

### G. Compositions Comprising Compounds of Formulae (I) and (II)

The disclosure further provides a composition comprising compounds of formulae (I) and (II):

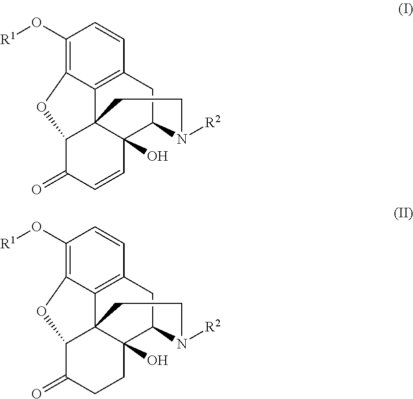

or (optionally pharmaceutically acceptable) salts or solvates thereof; where:
R¹ is —H, (C₁-C₇)alkyl, or an O-protecting group; and
R² is —H, —(C₂-C₄)alkenyl, —(C₂-C₇)alkyl, —(C₁-C₇)alkyl-(C₃-C₇)cycloalkyl, —CN, —C(═O)O—(C₁-C₆)alkyl, —C(═O)O-phenyl or an N-protecting group;
and where, in certain embodiments, the amount of the compound of formula (I) or a salt or a solvate thereof in the composition relative to the amount of the compound of formula (II) or a salt or a solvate thereof is less than about 200 ppm, less than about 150 ppm, less than about 100 ppm, less than about 50 ppm, less than about 40 ppm, less than about 35 ppm, less than about 25 ppm, less than about 10 ppm, or less than 5 ppm.

In a preferred embodiment, the amount of the compound of formula (I) or a salt or a solvate thereof relative to the amount of the compound of formula (II) or a salt or a solvate thereof in the product is less than about 40 ppm. In a more preferred embodiment, the amount of the compound of formula (I) or a salt or a solvate thereof relative to the amount of the compound of formula (II) or a salt or a solvate thereof in the product is less than about 35 ppm. In a more preferred embodiment, the amount of the compound of the compound of formula (I) is less than about 25 ppm. In an even more preferred embodiment, the amount of the compound of the compound of formula (I) is less than about 10 ppm. In a most preferred embodiment, the amount of the compound of the compound of formula (I) is less than 5 ppm. In certain embodiments, the disclosure provides said composition comprising compounds of formulae (I) and (II) or salts or solvates thereof as solid, in solution or as a suspension.

In certain embodiments, said composition is provided in its solid form, which in certain embodiments is its crystalline form.

The compositions of the disclosure comprising compounds of formulae (I) and (II) or a salt or a solvate thereof are obtainable or have been obtained by the processes described above.

The salts or solvates of the compositions comprising compounds of formulae (I) and (II) can be pharmaceutically acceptable salts or solvates. The counterions useful for forming pharmaceutically acceptable salts and the solvents useful for forming pharmaceutically acceptable solvates are known in the art.

In a preferred embodiment, R¹ is —H.

In a preferred embodiment, R² is —H.

In a preferred embodiment, R² is —(C₂-C₄)alkenyl or —(C₁-C₇)alkyl-(C₃-C₇)cycloalkyl.

In a more preferred embodiment, R² is —CH₂CH═CH₂ or —CH₂-cyclopropyl.

In a preferred composition of the disclosure, the compound of formula (I) is 14-hydroxynormorphinone (Impurity 1):

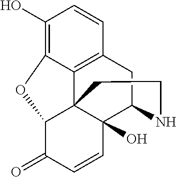 [SMILES: O=C1C=C[C@@]2(O)[C@H]3Cc4ccc(O)c5c4[C@@]2(CCN3)[C@H]1O5]

or a salt or a solvate thereof.

In this preferred composition of the disclosure, the compound of formula (II) is noroxymorphone (3):

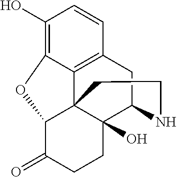 [SMILES: O=C1CC[C@@]2(O)[C@H]3Cc4ccc(O)c5c4[C@@]2(CCN3)[C@H]1O5]

or a salt or a solvate thereof.

In another preferred composition of the disclosure, the compound of formula (I) is 7,8-didehydronaloxone (Impurity 4):

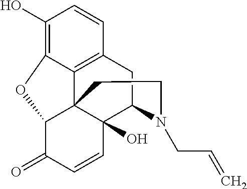 [SMILES: C=CCN1CC[C@]23c4c5ccc(O)c4O[C@H]2C(=O)C=C[C@@]3(O)[C@H]1C5]

or a salt or a solvate thereof.

In this preferred composition of the disclosure, the compound of formula (II) is naloxone (6):

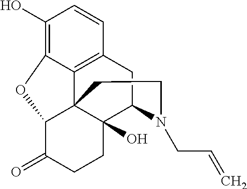 [SMILES: C=CCN1CC[C@]23c4c5ccc(O)c4O[C@H]2C(=O)CC[C@@]3(O)[C@H]1C5]

or a salt or a solvate thereof.

In another preferred composition of the disclosure, the compound of formula (I) is 7,8-didehydronaltrexone (Impurity 6):

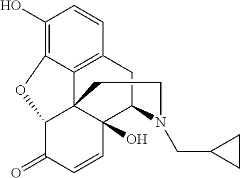 [SMILES: O=C1C=C[C@@]2(O)[C@H]3Cc4ccc(O)c5c4[C@@]2(CCN3CC2CC2)[C@H]1O5]

or a salt or a solvate thereof.

In this preferred composition of the disclosure, the compound of formula (II) is naltrexone (7):

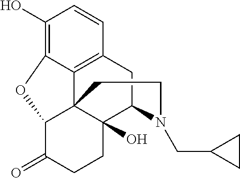 [SMILES: O=C1CC[C@@]2(O)[C@H]3Cc4ccc(O)c5c4[C@@]2(CCN3CC2CC2)[C@H]1O5]

or a salt or a solvate thereof.

In preferred embodiments, the composition comprising compounds of formulae (I) and (II) is 14-hydroxynormorphinone and noroxymorphone respectively, or the salts or solvates thereof.

In yet further preferred embodiments, the composition comprising compounds of formulae (I) and (II) is 7,8-didehydronaloxone and naloxone respectively, or the salts or solvates thereof.

In yet further preferred embodiments, the composition comprising compounds of formulae (I) and (II) is 14-hydroxynaltrexone and naltrexone respectively, or the salts or solvates thereof.

Said composition comprising compounds of formulae (I) and (II) can be in solid or in liquid form. In certain embodiments, the composition is a solid. In certain embodiments, the composition is a precipitate containing the compound of formula (II) as disclosed in the present application.

In certain embodiments, the salt of a compound of formula (I) is a compound of formula (Ia), the compound of formula (II) is a compound of formula (IIa), or the salt of a compound of formula (I) is a compound of formula (Ia) and the compound of formula (II) is a compound of formula (IIa):

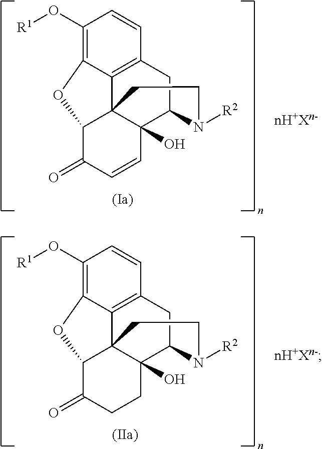

where:
R¹ is —H, (C₁-C₇)alkyl, or an O-protecting group;
R² is —H, —(C₂-C₄)alkenyl, —(C₂-C₇)alkyl, —(C₁-C₇)alkyl-(C₃-C₇)cycloalkyl, —CN, —C(═O)O—(C₁-C₆)alkyl, —C(═O)O-phenyl or an N-protecting group;

Xⁿ⁻ is an anion selected from the group consisting of I⁻, F⁻, valerate, acetate, meconate, salicylate, barbiturate, Br⁻, succinate, tartrate, maleate, fumarate, citrate, NO₃⁻, Cl⁻, HSO₄⁻, SO₄²⁻, methanesulfonate, tosylate, trifluoroacetate, H₂PO₄²⁻, HPO₄²⁻, [(NH₄)HPO₄]⁻, oxalate, perchlorate, H₃CC(O)O⁻, HC(O)O⁻, and mixtures thereof; and
n is 1 or 2.

In a preferred embodiment, Xⁿ⁻ is HSO₄⁻, SO₄²⁻, H₂PO₄⁻, HPO₄²⁻, [(NH₄)HPO₄]⁻, H₃CC(O)O⁻, HC(O)O⁻, or mixtures thereof. Even more preferably, Xⁿ⁻ is H₂PO₄⁻, HPO₄²⁻, [(NH₄)HPO₄]⁻, H₃CC(O)O⁻, HC(O)O⁻, or mixtures thereof. Most preferably, Xⁿ⁻ is H₂PO₄⁻, HPO₄²⁻, [(NH₄)HPO₄]⁻, or mixtures thereof.

In a preferred embodiment, parameter “n” is 2. In a more preferred embodiment parameter “n” is 1.

In a preferred embodiment, the compound of formula (Ia), the compound of formula (IIa), or the compound of formula (Ia) and the compound of formula (IIa) is obtained by adding an acid H⁺ₙXⁿ⁻ to the reaction composition. In a more preferred embodiment, the acid is H₃PO₄.

Any combination of elements of these groups defined for R¹, R², Xⁿ⁻ and n is also encompassed by the definitions of formulae (I) and (II).

In certain embodiments, the composition comprises compounds of formulae (Ia) and (IIa), where the compound of (Ia) is selected from one of the following:

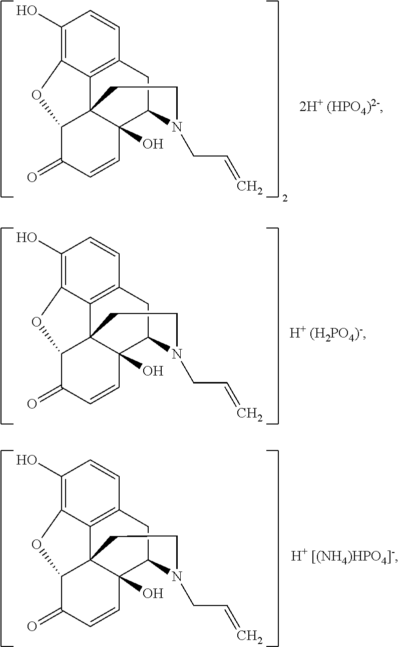

or a solvate thereof.

In certain embodiments, the composition comprises compounds of formulae (Ia) and (IIa), where the compound of (Ia) is selected from one of the following:

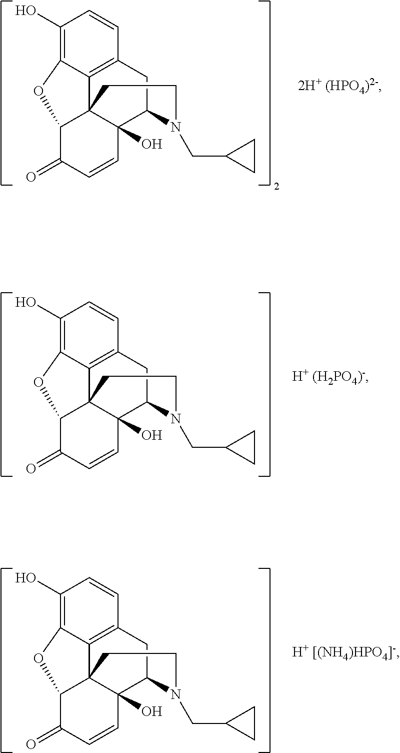

or a solvate thereof.

In a preferred embodiment the composition comprises compounds of formulae (Ia) and (IIa), where the compound of formula (Ia) is:

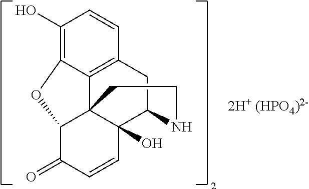

or a solvate thereof.

In a preferred embodiment the composition comprises compounds of formulae (Ia) and (IIa), where the compound of formula (Ia) is:

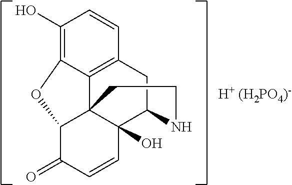

or a solvate thereof.

In a preferred embodiment the composition comprises compounds of formulae (Ia) and (IIa), where the compound of formula (Ia) is:

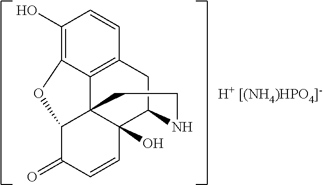

or a solvate thereof.

In certain embodiments, the composition comprises compounds of formulae (Ia) and (IIa), where the compound of (IIa) is selected from one of the following:

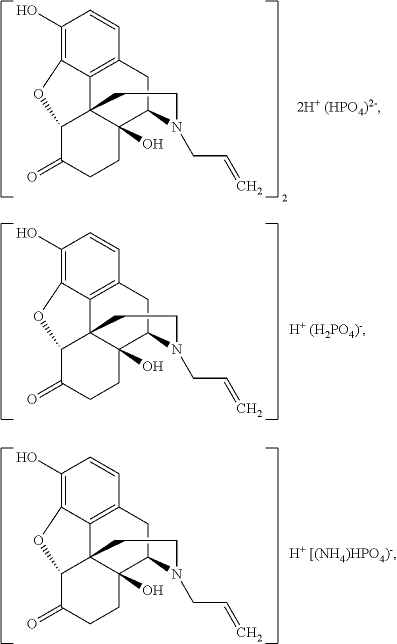

or a solvate thereof.

In certain embodiments, the composition comprises compounds of formulae (Ia) and (IIa), where the compound of (IIa) is selected from one of the following:

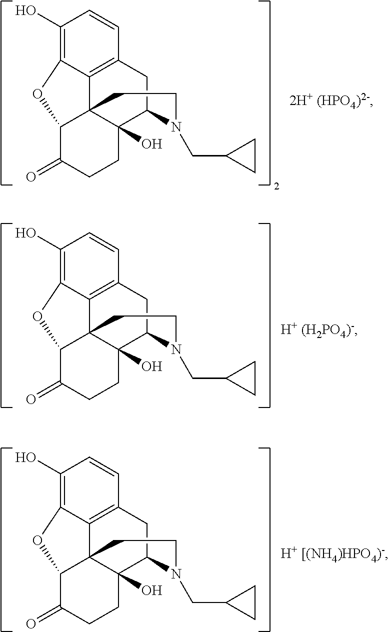

or a solvate thereof.

In a preferred embodiment the composition comprises compounds of formulae (Ia) and (IIa), where the compound of formula (IIa) is:

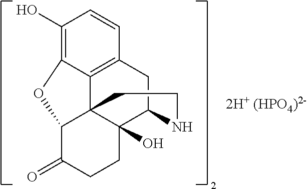

or a solvate thereof.

In a preferred embodiment the composition comprises compounds of formulae (Ia) and (IIa), where the compound of formula (IIa) is:

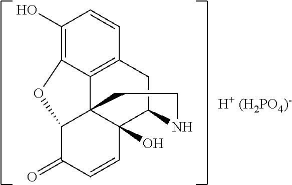

or a solvate thereof.

In a preferred embodiment the composition comprises compounds of formulae (Ia) and (IIa), where the compound of formula (IIa) is:

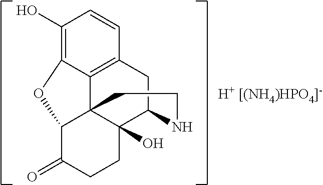

or a solvate thereof.

In certain embodiments, the composition comprises compounds of formulae (Ia) and (IIa), where the compound of (IIa) is:

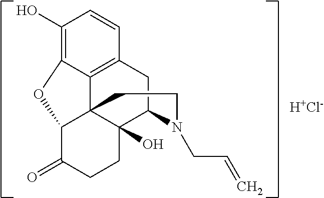

or a solvate thereof.

In certain embodiments, the composition comprises compounds of formulae (Ia) and (IIa), where the compound of (IIa) is:

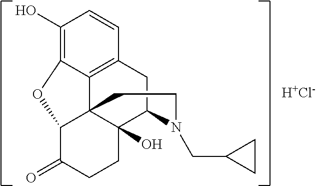

or a solvate thereof.

In certain embodiments, the composition comprising at least one of the compounds of formulae (I), (Ia), (II), or (IIa) is a hydrate of the compound of formulae (I), (Ia), (II), or (IIa).

In a preferred embodiment, the hydrate is a hydrate containing from about 0.5 to about 5.0 water molecules per molecule of the compound of formula (I), the compound of formula (Ia), the compound of formula (II), the compound of formula (IIa), or at least one of the compounds of formulae (I), (Ia), (II), and (IIa).

In a more preferred embodiment, the hydrate is a monohydrate, dihydrate or trihydrate. In an even more preferred embodiment, the hydrate is a dihydrate.

In certain embodiments, the composition comprising compounds of formulae (I) and (II) or the (optionally pharmaceutically acceptable) salts or solvates thereof further comprises a compound of formula (III):

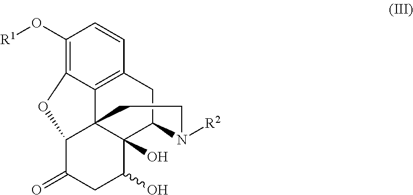

where:
R¹ is —H, (C₁-C₇)alkyl, or an O-protecting group; and
R² is —H, —(C₂-C₄)alkenyl, —(C₂-C₇)alkyl, —(C₁-C₇)alkyl-(C₃-C₇)cycloalkyl, —CN, —C(═O)O—(C₁-C₆)alkyl, —C(═O)O-phenyl or an N-protecting group.

In certain embodiments, the compound of formula (III) is:

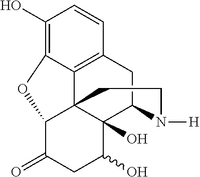 [SMILES: O=C1CC(O)[C@@]2(O)[C@H]3Cc4ccc(O)c5c4[C@@]2(CCN3)[C@H]1O5]

or a salt or a solvate thereof.

In certain embodiments, the compound of formula (III) may be present in the product in the form of its free base, or in the form of its salt of formula (IIIa):

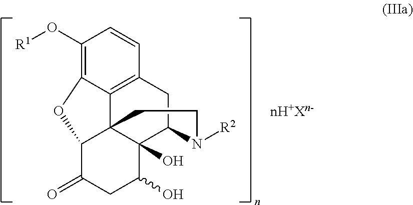

or a solvate thereof; where:
R¹ is —H, (C₁-C₇)alkyl, or an O-protecting group;
R² is —H, —(C₂-C₄)alkenyl, —(C₂-C₇)alkyl, —(C₁-C₇)alkyl-(C₃-C₇)cycloalkyl, —CN, —C(═O)O—(C₁-C₆)alkyl, —C(═O)O-phenyl or an N-protecting group;

Xⁿ⁻ is an anion selected from the group consisting of I⁻, F⁻, valerate, acetate, meconate, salicylate, barbiturate, Br⁻, succinate, tartrate, maleate, fumarate, citrate, NO₃⁻, Cl⁻, HSO₄⁻, SO₄²⁻, methanesulfonate, tosylate, trifluoroacetate, H₂PO₄⁻, HPO₄²⁻, [(NH₄)HPO₄]⁻, oxalate, perchlorate,
H₃CC(O)O⁻, HC(O)O⁻, and mixtures thereof; and
n is 1 or 2.

In certain embodiments, the amount of the compound of formula (III) or a salt or a solvate thereof relative to the amount of the compound of formula (II) or a salt or a solvate thereof in the product is less than about 2500 ppm, less than about 2250 ppm, less than about 2000 ppm, less than about 1750 ppm, less than about 1500 ppm, less than about 1250 ppm, less than about 1000 ppm, less than about 750 ppm, less than about 500 ppm, less than about 400 ppm, less than about 300 ppm, less than about 275 ppm, less than about 250 ppm, less than about 225 ppm, less than about 200 ppm, less than about 175 ppm, less than about 150 ppm (e.g., the amount of 8-hydroxynoroxymorphone is about 150 ppm relative to the amount of noroxymorphone), less than about 125 ppm, less than about 100 ppm, less than about 90 ppm, less than about 80 ppm, less than about 70 ppm, less than about 60 ppm, less than about 50 ppm, less than about 40 ppm, less than about 35 ppm, less than about 30 ppm, less than about 20 ppm or less than about 10 ppm.

In a preferred embodiment, the amount of the compound of formula (III) or a salt or a solvate thereof relative to the amount of the compound of formula (II) or a salt or a solvate thereof in the product is less than about 1500 ppm, less than about 1250 ppm, less than about 1000 ppm, less than about 750 ppm, less than about 500 ppm, less than about 400 ppm, less than about 300 ppm less than about 250 ppm, less than about 200 ppm, or less than about 150 ppm.

In certain embodiments, the compound of formula (II) is noroxymorphone or a salt or a solvate thereof, the compound of formula (III) is 8-hydroxynoroxymorphone or a salt or a solvate thereof and the amount of 8-hydroxynoroxymorphone or a salt or a solvate thereof relative to the amount of noroxymorphone in the composition is less than about 2500 ppm, less than about 2250 ppm, less than about 2000 ppm, less than about 1750 ppm, less than about 1500 ppm, less than about 1250 ppm, less than about 1000 ppm, less than about 750 ppm, less than about 500 ppm, less than about 400 ppm, less than about 300 ppm, less than about 275 ppm, less than about 250 ppm, less than about 225 ppm, less than about 200 ppm, less than about 175 ppm, less than about 150 ppm, less than about 125 ppm, less than about 100 ppm, less than about 90 ppm, less than about 80 ppm, less than about 70 ppm, less than about 60 ppm, less than about 50 ppm, less than about 40 ppm, less than about 35 ppm, less than about 30 ppm, less than about 20 ppm or less than about 10 ppm. In a preferred embodiment, the amount of the 8-hydroxynoroxymorphone or a salt or a solvate thereof relative to the amount of the noroxymorphone or a salt or a solvate thereof in the product is less than about 1500 ppm, less than about 1250 ppm, less than about 1000 ppm, less than about 750 ppm or less than about 500 ppm. In a more preferred embodiment, the amount of 8-hydroxynoroxymorphone or a salt or a solvate thereof is less than about 400 ppm, less than about 300 ppm, less than about 250 ppm, less than about 200 ppm or less than about 150 ppm.

In certain embodiments, the compound of formula (II) is naloxone or a salt or a solvate thereof, the compound of formula (III) is 8-hydroxynaloxone or a salt or a solvate thereof and the amount of 8-hydroxynaloxone or a salt or a solvate thereof relative to the amount of naloxone in the composition is less than about 2500 ppm, less than about 2250 ppm, less than about 2000 ppm, less than about 1750 ppm, less than about 1500 ppm, less than about 1250 ppm, less than about 1000 ppm, less than about 750 ppm, less than about 500 ppm, less than about 400 ppm, less than about 300 ppm, less than about 275 ppm, less than about 250 ppm, less than about 225 ppm, less than about 200 ppm, less than about 175 ppm, less than about 150 ppm, less than about 125 ppm, less than about 100 ppm, less than about 90 ppm, less than about 80 ppm, less than about 70 ppm, less than about 60 ppm, less than about 50 ppm, less than about 40 ppm, less than about 35 ppm, less than about 30 ppm, less than about 20 ppm or less than about 10 ppm.

In certain embodiments, the compound of formula (II) is naltrexone or a salt or a solvate thereof, the compound of formula (III) is 8-hydroxynaltrexone or a salt or a solvate thereof and the amount of 8-hydroxynaltrexone or a salt or a solvate thereof relative to the amount of naltrexone in the composition is less than about 2500 ppm, less than about 2250 ppm, less than about 2000 ppm, less than about 1750 ppm, less than about 1500 ppm, less than about 1250 ppm, less than about 1000 ppm, less than about 750 ppm, less than about 500 ppm, less than about 400 ppm, less than about 300 ppm, less than about 275 ppm, less than about 250 ppm, less than about 225 ppm, less than about 200 ppm, less than about 175 ppm, less than about 150 ppm, less than about 125 ppm, less than about 100 ppm, less than about 90 ppm, less than about 80 ppm, less than about 70 ppm, less than about 60 ppm, less than about 50 ppm, less than about 40 ppm, less than about 35 ppm, less than about 30 ppm, less than about 20 ppm or less than about 10 ppm.

In a preferred embodiment, the amount of the 8-hydroxynaloxone or a salt or a solvate thereof relative to the amount of the naloxone or a salt or a solvate thereof in the product is less than about 1500 ppm, less than about 1250 ppm, less than about 1000 ppm, less than about 750 ppm, less than about 500 ppm, less than about 400 ppm, less than about 300 ppm, less than about 250 ppm, less than about 200 ppm or less than about 150 ppm.

In a preferred embodiment, the amount of the 8-hydroxynaltrexone or a salt or a solvate thereof relative to the amount of the naltrexone or a salt or a solvate thereof in the product is less than about 1500 ppm, less than about 1250 ppm, less than about 1000 ppm, less than about 750 ppm, less than about 500 ppm, less than about 400 ppm, less than about 300 ppm, less than about 250 ppm, less than about 200 ppm or less than about 150 ppm.

In certain embodiments, the compound of formula (II) is noroxymorphone or a salt or a solvate thereof, the compound of formula (III) is 8-hydroxynoroxymorphone or a salt or a solvate thereof, and the compound of formula (I) is 14-hydroxynormorphinone or a salt or a solvate thereof. The compound of formula (II) can be an noroxymorphone salt. In one embodiment, it can be noroxymorphone hydrogen phosphate.

In certain embodiments, the compound of formula (II) is naloxone or a salt or a solvate thereof, the compound of formula (III) is 8-hydroxynaloxone or a salt or a solvate thereof. The compound of formula (II) can be a naloxone salt. In one embodiment, it can be naloxone hydrogen phosphate.

In certain embodiments, the compound of formula (II) is naltrexone or a salt or a solvate thereof, the compound of formula (III) is 8-hydroxynaltrexone or a salt or a solvate thereof. The compound of formula (II) can be a naltrexone salt. In one embodiment, it can be naltrexone hydrogen phosphate.

In certain embodiments, the composition comprising compounds of formulae (I) and (II) or the (optionally pharmaceutically acceptable) salts or solvates thereof additionally comprises a compound of formula (III). In certain embodiments, said composition comprises a combined amount of compound of formula (I) and compound of formula (III) which is less than about 4000 ppm, less than about 3500 ppm, less than about 3000 ppm, less than about 2750 ppm, less than about 2500 ppm, less than about 2250 ppm, less than about 2000 ppm, less than about 1750 ppm, less than about 1500 ppm, less than about 1250, less than about 1000 ppm, less than about 750 ppm, less than about 500 ppm, less than about 400 ppm, less than about 300 ppm, less than about 275 ppm, less than about 250 ppm, less than about 225 ppm, less than about 200 ppm, less than about 175 ppm, less than about 150 ppm, less than about 125 ppm, less than about 100 ppm, less than about 90 ppm, less than about 80 ppm, less than about 70 ppm, less than about 60 ppm, less than about 50 ppm, less than about 40 ppm, less than about 35 ppm, less than about 30 ppm, less than about 20 ppm, or less than about 10 ppm relative to the amount of the compound of formula (II).

In a preferred embodiment, the composition comprising compounds of formulae (I) and (II) comprises a combined amount of compound of formula (I) and compound of formula (III), which is less than about 1500 ppm, less than about 1250 ppm, less than about 1000 ppm, less than about 750 ppm, less than about 500 ppm, less than about 400 ppm, less than about 300 ppm, less than about 250 ppm, less than about 200 ppm or less than about 150 ppm.

In certain embodiments, the compound of formula (II) in the composition is noroxymorphone or a salt or a solvate thereof, and the composition additionally comprises 14-hydroxynormorphinone or a salt or a solvate thereof and optionally 8-hydroxynoroxymorphone or a salt or a solvate thereof, where the amount of the 14-hydroxymorphinone is less than about 200 ppm, less than about 150 ppm, less than about 100 ppm, less than about 50 ppm, less than about 40 ppm, less than about 35 ppm, less than about 25 ppm, less than about 10 ppm, or less than about 5 ppm relative to the amount of noroxymorphone, and the amount of 8-hydroxynoroxymorphone is less than about 1500 ppm, less than about 1250, less than about 1000 ppm, less than about 750 ppm, less than about 500 ppm, less than about 400 ppm, less than about 300 ppm, less than about 250 ppm, less than about 200 ppm, or less than about 150 ppm relative to the amount of noroxymorphone. In certain embodiments, the compound of formula (II) is noroxymorphone free base.

In certain embodiments, the compound of formula (II) in the composition is naloxone or a salt or a solvate thereof, and the composition additionally comprises 7,8-didehydronaloxone or a salt or a solvate thereof and optionally 8-hydroxynaloxone or a salt or a solvate thereof, where the amount of the 7,8-didehydronaloxone is less than about 200 ppm, less than about 150 ppm, less than about 100 ppm, less than about 50 ppm, less than about 40 ppm, less than about 35 ppm, less than about 25 ppm, less than about 10 ppm or less than about 5 ppm relative to the amount of naloxone, and the amount of 8-hydroxynaloxone is less than about 1500 ppm, less than about 1250, less than about 1000 ppm, less than about 750 ppm, less than about 500 ppm, less than about 400 ppm, less than about 300 ppm, less than about 250 ppm, less than about 200 ppm, or less than about 150 ppm relative to the amount of naloxone. In certain embodiments, the compound of formula (II) is naloxone free base.

In certain embodiments, the compound of formula (II) in the composition is naltrexone or a salt or a solvate thereof, and the composition additionally comprises 7,8-didehydronaltrexone or a salt or a solvate thereof and optionally 8-hydroxynaltrexone or a salt or a solvate thereof, where the amount of the 7,8-didehydronaltrexone is less than about 200 ppm, less than about 150 ppm, less than about 100 ppm, less than about 50 ppm, less than about 40 ppm, less than about 35 ppm, less than about 25 ppm, less than about 10 ppm or less than about 5 ppm relative to the amount of naltrexone, and the amount of 8-hydroxynaltrexone is less than about 1500 ppm, less than about 1250, less than about 1000 ppm, less than about 750 ppm, less than about 500 ppm, less than about 400 ppm, less than about 300 ppm, less than about 250 ppm, less than about 200 ppm, or less than about 150 ppm relative to the amount of naltrexone. In certain embodiments, the compound of formula (II) is naltrexone free base.

In certain embodiments, other morphinan derivatives are contained in the composition comprising compounds of formulae (I) and (II) or a salt or a solvate thereof.

In preferred embodiments, the composition of the disclosure comprises an amount of the compound of formula (I) or a salt or a solvate thereof in the product which is less than about 200 ppm, less than about 100 ppm, preferably less than about 75 ppm, more preferably less than about 50 ppm, even more preferably less than about 25 ppm and most preferably less than about 10 ppm relative to the amount of the compound of formula (II) or a salt or a solvate thereof, and an amount of the compound of formula (IV) or a salt or a solvate thereof which is less than about 0.5 HPLC peak area ratio, preferably less than about 0.25 HPLC peak area ratio, and most preferably less than about 0.15 HPLC peak area ratio. The method for determining the HPLC peak area ratio can preferably be the same as or analogous to the method as shown in Example 1.2. herein below. The HPLC peak area ratio refers to the area under the peak corresponding to the compound of formula (IV) divided by the area under the peak corresponding to the compound of formula (II).

In certain preferred embodiment, the composition of the disclosure comprises 14-hydroxynoroxymorphinone as the compound of formula (I) or a salt or a solvate thereof, noroxymorphone as the compound of formula (II) or a salt or a solvate thereof, and 3,4,14-trihydroxymorphinan-6-one as the compound of formula (IV) or a salt or a solvate thereof, where the amount of 14-hydroxynormorphinone or a salt or a solvate thereof relative to the amount of the compound of noroxymorphone or a salt or a solvate thereof is less than about 200 ppm, preferably less than about 100 ppm, more preferably less than about 75 ppm, more preferably less than about 50 ppm, more preferably less than about 10 ppm and most preferably less than 5 ppm, and where the amount of and 3,4,14-trihydroxymorphinan-6-one or a salt or a solvate thereof is less than about 0.5 HPLC peak area ratio, preferably less than about 0.25 HPLC peak area ratio and most preferably less than about 0.15 HPLC peak area ratio of the composition. The method for determining the HPLC peak area ratio of 3,4,14-trihydroxymorphinan-6-one is preferably the same as the method as shown in Example 1.2. herein below. The HPLC peak area ratio refers to the area under the peak corresponding to 3,4,14-trihydroxymorphinan-6-one divided by the area under the peak corresponding to noroxymorphinone and where preferably the method for determining the ppm amount of 14-hydroxynormorphinone can be the same as shown in the HPLC Example 1.1. herein below.

In certain embodiments, the compound of formula (II) in the composition is noroxymorphone or naloxone or a salt or a solvate thereof, and the composition contains not only 8-hydroxynoroxymorphone or 8-hydroxynaloxone, 14-hydroxynormorphinone or 7,8-didehydronaloxone, or 8-hydroxynoroxymorphone or 8-hydroxynaloxone and 14-hydroxynormorphinone or 7,8-didehydronaloxone as described above, but also in addition one or more of the following compounds: noroxymorphone-N-oxide, 6α-noroxymorphol (also known as 6alpha-noroxymorphol), 10-hydroxyoxymorphone, 10-ketooxymorphone, oxymorphone, hydromorphone, and hydroxydihydromorphine.

In certain embodiments, the compound of formula (II) in the composition is noroxymorphone or naltrexone or a salt or a solvate thereof, and the composition contains not only 8-hydroxynoroxymorphone or 8-hydroxynaltrexone, 14-hydroxynormorphinone or 7,8-didehydronaltrexone, or 8-hydroxynoroxymorphone or 8-hydroxynaltrexone and 14-hydroxynormorphinone or 7,8-didehydronaltrexone as described above, but also in addition one or more of the following compounds: noroxymorphone-N-oxide, 6α-noroxymorphol (also known as 6alpha-noroxymorphol), 10-hydroxyoxymorphone, 10-ketooxymorphone, oxymorphone, hydromorphone, and hydroxydihydromorphine.

### H. Product-by-Process

In certain embodiments, the composition comprises compounds of formulae (I) and (II):

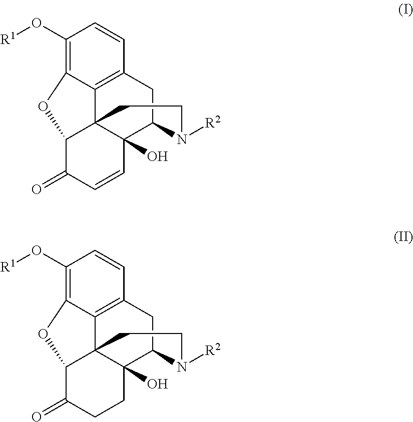

or a pharmaceutically acceptable salt or solvate thereof;
obtainable by the process for reducing the amount of a compound of formula (I) or a salt or a solvate thereof as defined herein above; where:
R¹ is —H, (C₁-C₇)alkyl, or an O-protecting group; and
R² is —H, —(C₂-C₄)alkenyl, —(C₂-C₇)alkyl, —(C₁-C₇)alkyl-(C₃-C₇)cycloalkyl, —CN, —C(═O)O—(C₁-C₆)alkyl, —C(═O)O-phenyl or an N-protecting group;
and where the amount of the compound of formula (I) in the composition relative to the amount of the compound of formula (II) is less than about 200 ppm, less than about 150 ppm, less than about 100 ppm, less than about 50 ppm, less than about 35 ppm, less than about 25 ppm, less than about 10 ppm, or less than 5 ppm.

In certain embodiments, the composition comprising compounds of formulae (I) and (II) obtainable by the process of the disclosure, additionally comprises a compound of formula (III):

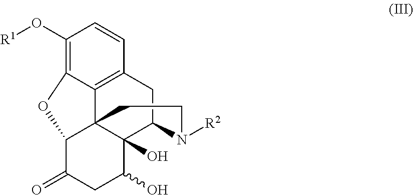

or a pharmaceutically acceptable salt or solvate thereof; where:
R¹ is —H, (C₁-C₇)alkyl, or an O-protecting group; and
R² is —H, —(C₂-C₄)alkenyl, —(C₂-C₇)alkyl, —(C₁-C₇)alkyl-(C₃-C₇)cycloalkyl, —CN, —C(═O)O—(C₁-C₆)alkyl, —C(═O)O-phenyl or an N-protecting group.

In certain embodiments, the composition comprising compounds of formulae (I) and (II) obtainable by a process of the disclosure comprises a combined amount of the compounds of formulae (I) and (III) or salts or solvates thereof in the composition relative to the amount of the compound of formula (II) or a salt or a solvate thereof, which is less than about 200 ppm, less than about 150 ppm, less than about 100 ppm, less than about 75 ppm, less than about 50 ppm, less than about 40 ppm, less than about 25 ppm, or less than about 10 ppm.

In certain embodiments, the composition comprising compounds of formulae (I) and (II) obtainable by a process of the disclosure comprises an amount of the compound of formula (I) or a salt or a solvate thereof in the product which is less than about 200 ppm, less than about 100 ppm, less than about 75 ppm, less than about 50 ppm, than about 25 ppm or less than about 10 ppm relative to the amount of the compound of formula (II) or a salt or a solvate thereof, and an amount of the compound of formula (IV) or a salt or a solvate thereof of less than about 0.5 HPLC peak area ratio, preferably less than about 0.25 HPLC peak area ratio and most preferably less than about 0.15 HPLC peak area ratio. The HPLC peak area ratio refers to the area under the peak corresponding to the compound of formula (IV) divided by the area under the peak corresponding to the compound of formula (II). Preferably, the ppm amount of compound of formula (I) or a salt or a solvate thereof is determined the same as or analogous to the HPLC method of Example 1.1. herein below, the method for determining the HPLC peak area ratio is the same as or analogous to the method as shown in Example 1.2. herein below, or the ppm amount of compound of formula (I) or a salt or a solvate thereof is determined the same as or analogous to the HPLC method of Example 1.1. herein below and the method for determining the HPLC peak area ratio is the same as or analogous to the method as shown in Example 1.2. herein below.

### I. Use of a Composition Comprising Compounds of Formulae (I) and (II)

I.1. Use in a Medicament

The disclosure further provides the use of a composition comprising compounds of formulae (I) and (II) or pharmaceutically acceptable salts or solvates thereof as a medicament.

For this use, the composition comprising compounds of formulae (I) and (II), or the pharmaceutically acceptable salts or solvates thereof, can be combined with at least one other morphinan derivative or pharmaceutically acceptable salt or solvate thereof in the medicament. For example, the at least one other morphinan derivative can be oxycodone or a pharmaceutically acceptable salt or solvate thereof or hydromorphone or a pharmaceutically acceptable salt or solvate thereof.

The medicament can be used for treating or preventing a medical condition selected from the group consisting of pain; addiction; cough; constipation; diarrhea; insomnia associated with or caused by pain, cough or addiction; depression associated with or resulting from pain, cough or addiction; or a combination of two or more of the foregoing conditions; in a particular embodiment, the condition is pain. In another embodiment, the medicament can be used for treating a medical condition selected from the group consisting of pain; addiction; cough; constipation; diarrhea; insomnia associated with or caused by pain, cough or addiction; depression associated with or resulting from pain, cough or addiction; or a combination of two or more of the foregoing conditions; in a particular embodiment, the condition is pain. In another embodiment, the medicament can be used for preventing a medical condition selected from the group consisting of pain; addiction; cough; constipation; diarrhea; insomnia associated with or caused by pain, cough or addiction; depression associated with or resulting from pain, cough or addiction; or a combination of two or more of the foregoing conditions; in a particular embodiment, the condition is pain.

In a preferred embodiment, the composition for use as a medicament comprises naloxone or a pharmaceutically acceptable salt or solvate thereof and 7,8-didehydronaloxone or a pharmaceutically acceptable salt or solvate thereof, where the amount of the 7,8-didehydronaloxone is less than about 200 ppm, less than about 150 ppm, less than about 100 ppm, less than about 50 ppm, less than about 40 ppm, less than about 35 ppm, less than about 25 ppm, less than about 10 ppm or less than 5 ppm relative to the amount of naloxone. More preferably, said composition comprising naloxone or a pharmaceutically acceptable salt or solvate thereof and 7,8-didehydronaloxone or a pharmaceutically acceptable salt or solvate thereof can be used in the treatment of opioid receptor agonist-induced bowel dysfunction, such as opioid receptor agonist-induced constipation, urinary retention, abdominal cramping, or gastroesophageal reflux; constipation; an opioid receptor agonist-induced depression or an opioid receptor agonist-induced overdose including breath depression, depression of the central nervous system and hypotension; prevention of opioid receptor agonist abuse; side effects of such opioid receptor agonist treatment such as anti-analgesia, hyperalgesia, hyperexcitability, physical dependence, or tolerance or a combination thereof. In one embodiment, said composition is used to counteract respiratory and other central nervous system depression in newborn resulting from the administration of analgesics to the mother during childbirth. In one embodiment said composition can be used as an adjunctive agent to increase blood pressure in the management of septic shock. In one preferred embodiment, the composition comprising naloxone or a pharmaceutically acceptable salt or solvate thereof and 7,8-didehydronaloxone or a pharmaceutically acceptable salt or solvate thereof can further comprise an additional morphinan derivative, preferably an opioid receptor agonist such as oxycodone or hydromorphone or pharmaceutically acceptable salts or solvates thereof.

In another embodiment, the composition for use as a medicament comprises naltrexone or a pharmaceutically acceptable salt or solvate thereof and 7,8-didehydronaltrexone or a pharmaceutically acceptable salt or solvate thereof which is less than about 200 ppm, less than about 150 ppm, less than about 100 ppm, less than about 50 ppm, less than about 35 ppm, less than about 25 ppm, less than about 10 ppm or less than about 5 ppm relative to the amount of naltrexone. More preferably, said composition comprising naltrexone or a pharmaceutically acceptable salt or solvate thereof and 7,8-didehydronaltrexone or a pharmaceutically acceptable salt or solvate thereof didehydronaltrexone or a pharmaceutically acceptable salt or solvate thereof is used in the treatment of addictions such as alcohol addiction or narcotics addiction such as opioid receptor agonist addiction. In one preferred embodiment, the composition comprising naloxone or a pharmaceutically acceptable salt or solvate thereof and 7,8-didehydronaloxone or a pharmaceutically acceptable salt or solvate thereof or a pharmaceutically acceptable salt or solvate thereof can further comprise an additional morphinan derivative, preferably an opioid receptor agonist such as oxycodone or hydromorphone or pharmaceutically acceptable salts or solvates thereof.

The disclosure also provides a method for treating an animal, preferably a mammal (e.g., a human) using the composition comprising compounds of formulae (I) and (II) or the pharmaceutically acceptable salts or solvates thereof. Said treatment can be of any medical condition which is conventionally treated by administration of the composition described above to an animal, preferably a mammal (e.g., a human), including those conditions listed above.

For a method of treatment of the disclosure, an effective amount of a composition comprising compounds of formulae (I) and (II) or a pharmaceutically acceptable salt or solvate thereof is generally administered to an animal in need thereof. For this method of treatment, the animal can be selected as a mammal. The mammal is generally a human or a companion animal (e.g., a dog or cat).

In a preferred embodiment, the method of treatment comprises the step of administering a composition comprising naloxone or a pharmaceutically acceptable salt or solvate thereof and 7,8-didehydronaloxone or a pharmaceutically acceptable salt or solvate thereof, where the amount of the 7,8-didehydronaloxone is less than about 200 ppm, less than about 150 ppm, less than about 100 ppm, less than about 50 ppm, less than about 40 ppm, less than about 35 ppm, less than about 25 ppm, less than about 10 ppm or less than 5 ppm relative to the amount of naloxone. More preferably, said composition comprising naloxone or a pharmaceutically acceptable salt or solvate thereof and 7,8-didehydronaloxone or a pharmaceutically acceptable salt or solvate thereof can be used in the treatment of opioid receptor agonist-induced bowel dysfunction, such as opioid receptor agonist-induced constipation, urinary retention, abdominal cramping, or gastroesophageal reflux; constipation; an opioid receptor agonist-induced depression or an opioid receptor agonist-induced overdose including breath depression, depression of the central nervous system and hypotension; prevention of opioid receptor agonist abuse; side effects of such opioid receptor agonist treatment such as anti-analgesia, hyperalgesia, hyperexcitability, physical dependence, or tolerance or a combination thereof. In one embodiment, said composition is used to counteract respiratory and other central nervous system depression in newborn resulting from the administration of analgesics to the mother during childbirth. In one embodiment said composition can be used as an adjunctive agent to increase blood pressure in the management of septic shock. In one preferred embodiment, the composition comprising naloxone or a pharmaceutically acceptable salt or solvate thereof and 7,8-didehydronaloxone or a pharmaceutically acceptable salt or solvate thereof can further comprise an additional morphinan derivative, preferably an opioid receptor agonist such as oxycodone or hydromorphone or a pharmaceutically acceptable salt or solvate thereof.

In another preferred embodiment, the method of treatment comprises administering a composition comprising naltrexone or a pharmaceutically acceptable salt or solvate thereof and 7,8-didehydronaltrexone or a pharmaceutically acceptable salt or solvate thereof, where the amount of 7,8-dehydronaltrexone is less than about 200 ppm, less than about 150 ppm, less than about 100 ppm, less than about 50 ppm, less than about 40 ppm, less than about 35 ppm, less than about 25 ppm, less than about 10 ppm or less than about 5 ppm relative to the amount of naltrexone. More preferably, said composition comprising naltrexone or a pharmaceutically acceptable salt or solvate thereof and 7,8-didehydronaltrexone or a pharmaceutically acceptable salt or solvate thereof. is used in the treatment of addictions such as alcohol addiction or narcotics addiction such as opioid receptor agonist addiction. In one preferred embodiment, the composition comprising naltrexone or a pharmaceutically acceptable salt or solvate thereof and 7,8-didehydronaltrexone or a pharmaceutically acceptable salt or solvate thereof or a pharmaceutically acceptable salt or solvate thereof can further comprise an additional morphinan derivative, preferably an opioid receptor agonist such as oxycodone or hydromorphone or a pharmaceutically acceptable salt or solvate thereof.

In one embodiment, the composition comprising naltrexone or a pharmaceutically acceptable salt or solvate thereof and 7,8-didehydronaltrexone or a pharmaceutically acceptable salt or solvate thereof or a pharmaceutically acceptable salt or solvate thereof can further comprise an additional active pharmaceutical ingredient (“API”), such as bupropion or a pharmaceutically acceptable salt thereof.

I.2. Other Uses

The composition comprising compounds of formulae (I) and (II) or an (optionally pharmaceutically acceptable) salt or solvate thereof can also be used as follows.

In certain embodiments, the composition comprising compounds of formulae (I) and (II) or (optionally pharmaceutically acceptable) salts or solvates thereof is used as an intermediate or starting material for preparing another salt or solvate of said composition comprising compounds of formulae (I) and (II), e.g., for preparing a first morphinan derivative or a pharmaceutically acceptable salt thereof. For example, when the composition comprising compounds of formulae (I) and (II) is a composition comprising 14-hydroxynormorphinone and noroxymorphone or a salt or a solvate thereof, respectively, said composition can be used for preparing a composition comprising naloxone or naltrexone or salts or solvates thereof, e.g., naloxone hydrochloride or naltrexone hydrochloride. Processes for preparing such other salts or solvates which involve a process or composition as described above in the detailed description are also embodiments of the disclosure.

In certain embodiments, the composition comprising compounds of formulae (I) and (II) or (optionally pharmaceutically acceptable) salt or solvate thereof is used as an intermediate or starting material for preparing a medicament comprising at least one other morphinan derivative or a pharmaceutically acceptable salt or solvate thereof or a prodrug thereof, or for preparing a medicament containing the composition comprising compounds of formulae (I) and (II) or pharmaceutically acceptable salts or solvates thereof, or containing at least one other morphinan derivative or a pharmaceutically acceptable salt or solvate thereof. For example, when the compounds of formulae (I) and (II) or a salt or a solvate thereof are 14-hydroxynormorphinone and noroxymorphone or salts or solvates thereof, respectively, such a composition can be used as starting material for preparing naloxone or a salt or a solvate thereof. In one embodiment, the at least one other morphinan derivative can be selected from the group consisting of oxycodone or a pharmaceutically acceptable salt or solvate thereof or hydromorphone or a pharmaceutically acceptable salt or solvate thereof. Processes for preparing a medicament comprising said other morphinan derivatives which involve a process or a composition as described above in the detailed description are also embodiments of the disclosure.

In certain embodiments, the first morphinan derivative is naloxone or a salt or a solvate thereof. In a preferred embodiment, the first morphinan derivative is naloxone hydrochloride or a solvate thereof.

In certain embodiments, the first morphinan derivative is naloxone or a salt of solvate thereof, where the amount of 7,8-didehydronaloxone or a salt or a solvate thereof relative to the amount of naloxone or a salt or a solvate thereof in the product is less than about 200 ppm, less than about 150 ppm, less than about 100 ppm, less than about 75 ppm, less than about 50 ppm, less than about 40 ppm, less than about 35 ppm, less than about 25 ppm, less than about 10 ppm, less than about 5 ppm or less than about 1 ppm.

In certain embodiments, the first morphinan derivative is naltrexone or a salt or a solvate thereof. In a preferred embodiment, the first morphinan derivative is naltrexone hydrochloride or a solvate thereof.

In certain embodiments, the first morphinan derivative is naltrexone or a salt or a solvate thereof, where the amount of 7,8-didehydronaltrexone or a salt or a solvate thereof relative to the amount of naltrexone or a salt or a solvate thereof in the product is less than about 200 ppm, less than about 150 ppm, less than about 100 ppm, less than about 75 ppm, less than about 50 ppm, less than about 40 ppm, less than about 35 ppm, less than about 25 ppm, less than about 10 ppm or less than 5 ppm.

### EXAMPLES

The following examples are set forth to assist in understanding the invention and should not be construed as specifically limiting the invention described and claimed herein. Such variations of the invention, including the substitution of all equivalents now known or later developed, that would be within the purview of those skilled in the art, and changes in formulation or changes in experimental design, are to be considered to fall within the scope of the invention incorporated herein.

### Example 1

### Determination of Impurities in Morphinans by HPLC

The following non-limiting examples illustrate the determination, by HPLC, of various impurities, e.g., compounds of formulae (I), (III), (IV), (V), and/or (VI), in certain morphinans.

### Example 1.1

### Determination of 14-Hydroxynormorphinone and 8-Hydroxynoroxymorphone in Noroxymorphone Preparation at ppm Levels

The following was the HPLC method used for the determination of 14-hydroxynormorphinone (e.g., a compound of formula (I), designated as “Impurity 1”) and 8-hydroxynoroxymorphone (e.g., a compound of formula (III), designated as “Impurity 2”) in noroxymorphone samples at ppm levels. The LOD was considered to be 5 ppm and the LOQ was considered to be 10 ppm with a relative standard deviation (“RSD”) of not more than ±20%. Quantitation of Impurity 1 was achieved by comparison to a 14-hydroxynormorphinone external standard. Quantitation of Impurity 2 was achieved by comparison to the 14-hydroxynormorphinone external standard in combination with the relative response factor (“RRF”) for Impurity 2.

The parameters of the HPLC method used are summarized in Table 1.

TABLE 1
HPLC Instrument Parameters

| | |
|---|---|
| HPLC Column | Phenomenex Gemini NX C18, 3 μm, |
| | 250 × 4.6 mm |
| Detection Wavelength | 240 nm |
| Detector | Waters 2487 Dual Wavelength Detector |
| | (or equivalent) |
| Sample Concentration | 4.0 mg/mL Noroxymorphone in 0.85% |
| | Aqueous H₃PO₄ |
| Injection Volume | 20 μL |
| Column Temperature | 45-48° C. |
| Sample Temperature | About 25° C. |
| Mobile Phase A | 0.1% Aqueous Ammonium Hydroxide |
| | (about 10.7 pH) |
| Mobile Phase B | 0.1% Ammonium Hydroxide in Methanol |
| Mobile Phase C | Acetonitrile |

Further, the HPLC elution profile as summarized in Table 2 was used.

TABLE 2
HPLC Method Flow Rate and Gradient

| Time | Flow | Mobile | Mobile | Mobile |
|---|---|---|---|---|
| (min) | (mL/min) | Phase A (%) | Phase B (%) | Phase C (%) |
| 0.0 | 0.5 | 95.0 | 3.0 | 2.0 |
| 17.0 | 0.5 | 95.0 | 3.0 | 2.0 |
| 33.0 | 0.5 | 40.0 | 58.0 | 2.0 |
| 33.5 | 0.5 | 95.0 | 3.0 | 2.0 |
| 45.0 | 0.5 | 95.0 | 3.0 | 2.0 |

No artificial peaks were observed at the retention times of Impurity 1 and Impurity 2 in the chromatogram obtained from a blank injection. Impurity 2 had a retention time (“RT”) of about 8.0 min. Impurity 1 had a RT of about 11.1 min. If necessary, the amount of Mobile Phase B can be increased up to about 3.3% or can be decreased down to about 2.7% and the flow rate can be increased up to about 0.55 mL/min or can be decreased down to about 0.45 mL/min to obtain these desired RTs. The relative retention time (“RRT”) for the Impurity 2 peak compared to the Impurity 1 peak was about 0.72 (=8.0/11.1). The peak area for six repeated injections of the 14-hydroxynormorphinone working standard solution of 0.0002 mg/mL 14-hydroxynormorphinone (which would correspond to 50 ppm in a 4 mg/mL noroxymorphone sample) had a RSD of no more than 20%.

The RTs of Impurity 1 and Impurity 2 are shown below in Table 3.

TABLE 3
Typical Retention Times of Impurity 1 and Impurity 2

| Peak | Typical RT (min) |
|---|---|
| Impurity 1 | 11.1 ± 1.7 min |
| Impurity 2 | 8.0 ± 1.2 min |

The ppm amount of Impurity 1 in samples was calculated using the following equation:

$$ppm ( Impurity 1 ) = \frac{{A}_{S} \times {W}_{STD}}{{A}_{STD} \times {W}_{S}} \times P \qquad ( Equation 5 )$$

The ppm amount of Impurity 2 in samples was calculated using the following equation:

$$ppm ( Impurity 2 ) = \frac{{A}_{S} \times {W}_{STD}}{{A}_{STD} \times {W}_{S}} \times \frac{1}{RRF} \times P \qquad ( Equation 6 )$$

where:
A_{S}=Impurity 1 or Impurity 2 peak area for sample,
A_{STD}=average peak area for 14-hydroxynormorphinone standard injections,
W_{STD}=actual weight of 14-hydroxynormorphinone external standard in mg,
W_{S}=actual weight of sample in mg,
P=external standard purity in %, and
RRF=relative response factor of Impurity 2 compared to Impurity 1 (i.e., 0.88).

14-Hydroxynormorphinone external standards were prepared from commercially available materials, for example, obtainable from Noramco (Wilmington, Del.) or Mallinckrodt Pharmaceuticals (St. Louis, Mo.).

### Example 1.2

### HPLC Method to Determine the Retention Times of Several Impurities in Noroxymorphone

The following was the HPLC method used for the determination of other impurities in noroxymorphone. The LOD was considered to be 0.01% and the LOQ was considered to be 0.05% with an RSD of not more than ±2%. Quantitation of noroxymorphone was achieved by comparison to a noroxymorphone external standard.

The parameters of the HPLC method used are summarized in Table 4.

TABLE 4
HPLC Instrument Parameters

| | |
|---|---|
| HPLC Column | Phenomenex Gemini NX C18, 5 μm, |
| | 250 × 4.6 mm |
| Detection Wavelength | 225 nm |
| Detector | Waters 2695 HPLC with 996 PDA or |
| | Waters 2487 Dual Wavelength Detector |
| | (or equivalent) |
| Sample Concentration | 4.0 mg/mL Noroxymorphone, e.g., in About |
| | 0.085% to About 0.85% Aqueous H₃PO₄ |
| Injection Volume | 10 μL |
| Column Temperature | 40° C. |
| Sample Temperature | About 25° C. |
| Mobile Phase A | 25 mM Potassium Phosphate Buffer (pH 8.30)/ |
| | Methanol (90/10) |
| Mobile Phase B | 25 mM Potassium Phosphate Buffer (pH 8.30)/ |
| | Methanol (25/75) |

Further, the HPLC elution profile as summarized in Table 5 was used.

TABLE 5
HPLC Method Flow Rate and Gradient

| Time | Flow | Mobile | Mobile |
|---|---|---|---|
| (min) | (mL/min) | Phase A (%) | Phase B (%) |
| 0.0 | 1.0 | 100 | 0 |
| 5.0 | 1.0 | 100 | 0 |
| 45.0 | 1.0 | 0 | 100 |
| 46.0 | 1.0 | 100 | 0 |
| 50.0 | 1.0 | 100 | 0 |

No artificial peaks were observed at the retention times of the reporting analytes in the chromatogram obtained from a blank injection. The RT for noroxymorphone is 12.1 min (±10%). If necessary, at least one of the following alterations can be made to obtain this desired RT: the methanol ratio in Mobile Phase A can be increased up to about 12% or can be decreased down to about 9%, the flow rate can be decreased down to about 0.90 mL/min, or the temperature can be adjusted ±5° C. The peak area for five repeated injections of the noroxymorphone working standard solution had a RSD of not more than 2.0%.

The RTs of noroxymorphone and certain impurities are shown below in Table 6; 3,4,14-trihydroxymorphinan-6-one is designated as “Impurity 3” (e.g., a compound of formula (IV)).

TABLE 6
Typical Retention Times of Noroxymorphone
and Impurities Therein

| Peak | Typical RT (min) |
|---|---|
| Noroxymorphone | 12.1 ± 1.2 |
| Impurity 1 | 14.5 ± 1.4 |
| Impurity 2 | 7.9 ± 0.8 |
| Impurity 3 | 9.4 ± 0.9 |

The area under the HPLC peak corresponding to Impurity 3 was determined as was the area under the HPLC peak corresponding to noroxymorphone. The HPLC peak area ratio refers to and was determined from the area under the peak corresponding to Impurity 3 divided by the area under the peak corresponding to noroxymorphone.

### Example 1.3

### HPLC Method for the Determination of 7,8-Didehydronaloxone and 8-Hydroxynaloxone in Naloxone and Naloxone Hydrochloride Dihydrate at ppm Levels

The following was the HPLC method used for the determination of 7,8-didehydronaloxone (e.g., a compound of formula (I), designated as “Impurity 4”) and 8-hydroxynaloxone (e.g., a compound of formula (III), designated as “Impurity 5”) in naloxone and naloxone hydrochloride dihydrate samples at ppm levels. The LOD was considered to be 5 ppm and the LOQ was considered to be 10 ppm with an RSD of not more than ±20%. Quantitation of Impurity 4 was achieved by comparison to a 7,8-didehydronaloxone external standard. Quantitation of Impurity 5 was achieved by comparison to the 7,8-didehydronaloxone external standard in combination with the RRF for Impurity 5.

The parameters of the HPLC method used are summarized in Table 7.

TABLE 7
HPLC Instrument Parameters

| | |
|---|---|
| HPLC Column | Phenomenex Gemini C6-Phenyl, 3 μm, |
| | 150 × 4.6 mm |
| Detection Wavelength | 215 nm |
| Detector | Waters 2487 Dual Wavelength Detector |
| | (or equivalent) |
| Sample Concentration | 10.0 mg/mL Naloxone in 100% Methanol |
| Injection Volume | 10 μL |
| Column Temperature | 35° C. |
| Sample Temperature | About 25° C. |
| Mobile Phase A | 10 mM Ammonium Bicarbonate (pH 10.0) |
| Mobile Phase B | Methanol |

Further, the HPLC elution profile as summarized in Table 8 was used.

TABLE 8
HPLC Method Flow Rate and Gradient

| Time | Flow | Mobile | Mobile |
|---|---|---|---|
| (min) | (mL/min) | Phase A (%) | Phase B (%) |
| 0.0 | 0.5 | 59.0 | 41.0 |
| 2.0 | 0.5 | 59.0 | 41.0 |
| 52.0 | 0.5 | 56.0 | 44.0 |
| 52.1 | 1.0 | 20.0 | 80.0 |
| 60.0 | 1.0 | 20.0 | 80.0 |
| 60.1 | 0.5 | 59.0 | 41.0 |
| 65.0 | 0.5 | 59.0 | 41.0 |

No artificial peaks were observed at the retention times of the reporting analytes in the chromatogram obtained from a blank injection. Impurity 4 had a RT of about 33.6 min. Impurity 5 had a RT of about 23.1 min. If necessary, at least one of the following alterations can be made to obtain these desired RTs: the amount of Mobile Phase B can be increased up to about 44.3% or can be decreased down to about 38.3%, the flow rate can be increased up to about 0.55 mL/min, the column temperature can be adjusted by ±3° C., or the pH of Mobile Phase A can be increased up to about 10.20 or can be decreased down to about 9.95. The RRT for the Impurity 5 peak compared to the Impurity 4 peak was about 0.69 (=23.1/33.6). The peak area for six repeated injections of 0.0002 mg/mL 7,8-didehydronaloxone working standard solution (which would correspond to 50 ppm 7,8-didehydronaloxone in a 4 mg/mL naloxone sample) had an RSD of not more than 20%.

The RTs of Impurity 4 and Impurity 5 are shown below in Table 9.

TABLE 9
Typical Retention Times of Impurity 4 and Impurity 5

| Peak | Typical RT (min) |
|---|---|
| Impurity 4 | 33.6 ± 3.3 min |
| Impurity 5 | 23.1 ± 2.3 min |

The HPLC peak area ratio for Impurity 4 refers to and was determined from the area under the peak corresponding to Impurity 4 divided by the area under the peak corresponding to the major component, e.g., naloxone or naloxone hydrochloride dihydrate. The HPLC peak area ratio for Impurity 5 refers to and was determined from the area under the peak corresponding to Impurity 5 divided by the area under the peak corresponding to the major component, e.g., naloxone or naloxone hydrochloride dihydrate.

The ppm amount of Impurity 4 or Impurity 5 in samples relative to, e.g., naloxone hydrochloride dihydrate in those samples, was calculated using the following equation:

$$ppm ( Impurity 4 or Impurity 5 ) = \frac{{R}_{S} \times {C}_{STD}}{{R}_{STD} \times {W}_{S}} \times \frac{1}{RRF} \times 10000 \times \frac{399.87}{327.37} \qquad ( Equation 7 )$$

where:
R_{S}=Impurity 4 or Impurity 5 peak area for sample,
R_{STD}=average peak area for 7,8-didehydronaloxone standard injections,
C_{STD}=concentration of 7,8-didehydronaloxone external standard in μg/mL, corrected for purity,
W_{S}=actual weight of sample in mg,
10000=conversion factor for ppm,
RRF=relative response factor of Impurity 5 compared to Impurity 4 (i.e., 0.66),
399.87=molecular weight of naloxone hydrochloride dihydrate, and
327.37=molecular weight of naloxone.

7,8-Didehydronaloxone external standards were prepared from commercially available materials, for example, obtained from Cerilliant Corp. (Round Rock, Tex.) or Mallinckrodt Pharmaceuticals.

### Example 1.4

### HPLC Method for the Determination of 7,8-Didehydronaltrexone in Naltrexone at ppm Levels

The following was the HPLC method used for the determination of 7,8-didehydronaltrexone (e.g., a compound of formula (I), designated as “Impurity 6”) in naltrexone samples at ppm levels. The LOD is believed to be about 5 ppm and the LOQ is believed to be 10 ppm with an RSD of not more than ±20%. Quantitation of Impurity 6 was achieved by comparison to a 7,8-didehydronaloxone external standard in combination with the RRF for Impurity 6.

The parameters of the HPLC method used are summarized in Table 10.

TABLE 10
HPLC Instrument Parameters

| | |
|---|---|
| HPLC Column | Phenomenex Gemini C6-Phenyl, 3 μm, |
| | 150 × 4.6 mm |
| Detection Wavelength | 215 nm |
| Detector | Waters 2487 Dual Wavelength Detector |
| | (or equivalent) |
| Sample Concentration | 10.0 mg/mL Naltrexone in 100% Methanol |
| Injection Volume | 10 μL |
| Column Temperature | 35° C. |
| Sample Temperature | About 25° C. |
| Mobile Phase A | 10 mM Ammonium Bicarbonate (pH 10.0) |
| Mobile Phase B | Methanol |

Further, the HPLC elution profile as summarized in Table 11 was used.

TABLE 11
HPLC Method Flow Rate and Gradient

| Time | Flow | Mobile | Mobile |
|---|---|---|---|
| (min) | (mL/min) | Phase A (%) | Phase B (%) |
| 0.0 | 0.5 | 59.0 | 41.0 |
| 2.0 | 0.5 | 59.0 | 41.0 |
| 52.0 | 0.5 | 56.0 | 44.0 |
| 52.1 | 1.0 | 20.0 | 80.0 |
| 60.0 | 1.0 | 20.0 | 80.0 |
| 60.1 | 0.5 | 59.0 | 41.0 |
| 65.0 | 0.5 | 59.0 | 41.0 |

No artificial peaks were observed at the retention times of the reporting analytes in the chromatogram obtained from a blank injection. Impurity 6 had a RT of about 50.5 min. The peak area for six repeated injections of 0.0005 mg/mL 7,8-didehydronaloxone working standard solution (which would correspond to 50 ppm 7,8-didehydronaloxone in a 10 mg/mL naltrexone sample) had an RSD of not more than 20%.

The RTs of Impurity 6 is shown below in Table 12.

TABLE 12
Typical Retention Times of Impurity 6 and Impurity 4

| Peak | Typical RT (min) |
|---|---|
| Impurity 6 | 50.5 min |
| Impurity 4 | 33.6 ± 3.3 min |

The ppm amount of Impurity 6 in samples relative to e.g. naltrexone in those samples, was calculated using the following equation:

$$ppm ( Impurity 6 ) = \frac{{R}_{S} \times {C}_{STD}}{{R}_{STD} \times {W}_{S}} \times \frac{1}{RRF} \times 10000 \times \frac{339.39}{325.36} \qquad ( Equation 8 )$$

where:
R_{S}=Impurity 6 peak area for sample,
R_{STD}=average peak area for 7,8-didehydronaloxone standard injections,
C_{STD}=concentration of 7,8-didehydronaloxone external standard in μg/mL, corrected for purity,
W_{S}=actual weight of sample in mg,
10000=conversion factor for ppm,
RRF=relative response factor of Impurity 6 compared to Impurity 4 (estimated to be 1)
339.39=molecular weight of 7,8-didehydronaltrexone, and
325.36=molecular weight of 7,8-didehydronaloxone.

7,8-Didehydronaloxone external standards were prepared from commercially available materials, for example, obtained from Cerilliant Corp. (Round Rock, Tex.) or Mallinckrodt Pharmaceuticals.

### Example 2

### Noroxymorphone Starting Materials

Different batches of noroxymorphone were used as the starting material. The batches had different levels of impurities and also differed in color. The amount of the impurities 14-hydroxynormorphinone (Impurity 1) and 8-hydroxynoroxymorphone (Impurity 2) were determined by HPLC as described above in Example 1.1. The impurities in the different batches and their YIs are summarized below in Table 13.

TABLE 13
Levels of Impurities 1 and 2 in Noroxymorphone
Starting Material Batches

| | Impurity 1 | Impurity 2 | |
|---|---|---|---|
| Batch | (ppm)^{a} | (ppm)^{a} | YI |
| 1 | 803 | 670 | 152.4 |
| 2 | 934 | 763 | 130.8 |
| 3 | 985 | 865 | 116.6 |
| 4 | 1005 | 898 | 128.3 |
| 5 | 811 | 780 | 144.0 |
| 6 | 937 | 812 | 144.1 |
| 7 | 888 | 801 | 140.8 |
| 8 | 830 | 706 | 119.3 |

^{a}RSD is not more than ±20%

### Example 3

### Decolorization by Activated Carbon Treatment of Noroxymorphone Followed by Hydrogenation

The decolorizing agent used was activated carbon from Sigma-Aldrich (100-400 mesh, Darco KB-G) at a loading of 23 wt. % (on an “as is” basis) relative to the amount of crude noroxymorphone charged. In Examples 3.1., 3.1.1. and 3.2., each batch of noroxymorphone that was activated-carbon treated was then split into two portions. In one portion the noroxymorphone was immediately isolated and in the remaining portion the noroxymorphone was hydrogenated and then the product was isolated.

### Example 3.1

### Batch 5 as the Noroxymorphone Source

To a 500-mL jacketed reactor was charged water (303 mL) and 85% aqueous H₃PO₄ (96.4 g, 2.4 molar equivalents). The solution was heated to about 50° C. with agitation and noroxymorphone (347.6 mmol, Batch 5) was added in several portions. The resultant solution was not transparent. The solution was heated to 75° C. and activated carbon (Darco KB-G, 30.0 g) was charged in one bolus. The reaction mixture was heated to 90° C. and held at that temperature for about 4 hours. The hot reaction mixture was filtered through Whatman #1 filter paper and the carbon bed was rinsed with 260 mL of water to provide Filtrate 1 (795.1 g, about 715 mL).

One portion of Filtrate 1 (393.2 g) was carried through the salt-breaking procedure as follows. To a 1-L round bottom flask was charged 393.2 g of Filtrate 1. The pH of the solution was 1.9 at 23° C. The solution was stirred and heated to a temperature of 75° C. (pH 1 7) Ammonium hydroxide (28-30%, 94.5 g) was added dropwise over 45 minutes to the heated solution at 75° C. (pH 8.2). The mixture was cooled to 20° C. (pH 9.5). The solids were isolated by vacuum filtration. The solids were washed with water (4×100 mL/wash). The solids were dried at 80° C. under reduced pressure for about 16 hours. The dried solids (noroxymorphone, Solid 2, 50.0 g) were analyzed by HPLC as described above in Example 1.1. (Impurity 1=973 ppm; Impurity 2=936 ppm) and color (YI=16.8 at a concentration of 4 mg/mL).

The remaining portion of Filtrate 1 (401.9 g) was hydrogenated as follows. To a 300 mL pressure vessel was charged 199.4 g of Filtrate 1 (about 182 mL, about 25.0 g noroxymorphone) along with 2.5 g of 5% palladium on carbon (50% water wet, Johnson-Matthey). To a separate 300 mL pressure vessel was charged 202.5 g of Filtrate 1 (about 183 mL, about 25.4 g noroxymorphone) along with 2.5 g of 5% palladium on carbon (50% water wet, Johnson-Matthey). Both reactors were purged and then heated to 80° C., pressurized with hydrogen to 517 kPa and stirred for 18 hours. The catalyst was filtered off and the remaining material was rinsed with water (2×20 mL/rinse). The combined filtrates were re-circulated through the catalyst bed once resulting in a transparent solution with no visible particulates. The two post hydrogenation filtrates were combined (401.9 g) and charged into a 4-neck 1-L round bottom flask. The pH of the solution was 2.0 at a temperature of about 25° C. The solution was stirred and heated to a temperature of 75° C. Ammonium hydroxide (28-30%, 84.7 g) was added dropwise over 45 minutes at 75° C. (pH 8.3). The mixture was cooled to 22° C. (pH 9.6). The solids were isolated by vacuum filtration and then the solids were washed with water (4×100 mL/wash). The wet cake was allowed to dry under suction for about 1 hour. The solids were dried at 80° C. under reduced pressure for about 18 hours. The dried solids (noroxymorphone, Solid 3, 48.7 g) were analyzed by HPLC as described above in Example 1.1. (Impurity 1=20 ppm; Impurity 2=662 ppm) and color (YI=14.2 at a concentration of 4 mg/mL).

### Example 3.1.1

### Multiple Carbon Charges

To a 4-neck, 1-L round bottom flask was charged water (420.3 mL), 85% aqueous H₃PO₄ (77.5 g, 2.6 molar equivalents), noroxymorphone (261.5 mmol, Batch 5) and activated carbon (Darco KB-G, 13.4 g, 13 wt. %). The mixture was heated to 90° C. and held at that temperature for about 2 hours. The hot reaction mixture was filtered and the carbon bed was rinsed with 20 mL of water. The filtrate was charged back into the reactor along with activated carbon (Darco KB-G, 13.4 g; 13 wt %). The reaction mixture was heated to 90° C. and held at that temperature for about 2 hours. The hot reaction mixture was filtered and the carbon bed was rinsed with 20 mL of water. This yielded Filtrate 4 as an opaque solution (690.8 g, about 625 mL).

One portion of Filtrate 4 (314.5 g) was carried through the salt-breaking procedure as follows. To a 4-neck, 1-L round bottom flask was charged 314.5 g of the noroxymorphone filtrate (Filtrate 4). The pH of the solution was 1.4 at about 23° C. The solution was stirred and heated to a temperature of 75° C. At 75° C., the pH was 1.4. Aqueous ammonium hydroxide (28-30 wt %, 87.4 g, Sigma-Aldrich) was added dropwise over 45 minutes to the heated solution. The pH after base addition at 75° C. was approximately 8.6. The mixture was cooled to 20° C. and the measured pH was 10.0. The solids were isolated by vacuum filtration and washed with water (4×100 mL/wash). The solids were dried at 80° C. under reduced pressure for about 18 hours. The dried solids (noroxymorphone, Solid 5, 34.0 g) were analyzed by HPLC as described above in Example 1.1. (Impurity 1=981 ppm; Impurity 2=956 ppm).

The remaining portion of Filtrate 4 (354.3 g) was hydrogenated and worked-up analogously to the procedure described for Filtrate 1. The dried solids (noroxymorphone, Solid 6, 37.6 g) were analyzed by HPLC as described above in Example 1.1. (Impurity 1=22 ppm; Impurity 2=566 ppm) and color (YI=9.7 at a concentration of 4 mg/mL).

### Example 3.1.2

### Stressed Reaction Conditions

To a 500 mL jacketed reactor was charged water (400.0 mL), 85% aqueous H₃PO₄ (96.7 g, 3.2 molar equivalents), noroxymorphone (261.5 mmol, Batch 5) and activated carbon (Darco KB-G, 26.7 g). The mixture was heated to 90° C. and held at that temperature for about 60 hours. The hot reaction mixture was filtered and the carbon bed was rinsed with 360 mL of water. This yielded Filtrate 7 as an opaque solution (854.7 g).

One portion of Filtrate 7 (427.0 g) was worked-up analogously to the procedure described for Filtrate 1. The solids were dried at 80° C. under reduced pressure for about 18 hours. The dried solid noroxymorphone (Solid 8, 37.7 g) was analyzed by HPLC as described above in Example 1.1. (Impurity 1=868 ppm; Impurity 2=1019 ppm).

The remaining portion of Filtrate 7 (427.8 g) was hydrogenated as follows. To a 500 mL pressure vessel was charged 217.6 g of Filtrate 7 (about 201 mL, about 19.9 g noroxymorphone) along with 1.0 g of a 10% palladium on carbon (50% water wet, Evonik Type E101 NE/W). The reactor was assembled. The catalyst was filtered off and rinsed with water (2×20 mL/rinse). The combined filtrates were re-circulated through the catalyst bed once resulting in a transparent solution with no visible particulates. The filtrate (301.2 g) was charged into a 4-neck 1-L round bottom flask and stirred and heated to a temperature of 75° C. (pH 1.5). Aqueous ammonium hydroxide (28-30 wt %) was added dropwise over 45 minutes at 75° C. (pH 8.5). The mixture was cooled to about 22° C. (pH 9.7). The solids were isolated by vacuum filtration. The solids were washed with water (4×100 mL/wash). The wet cake was allowed to dry under suction for about 1 hour followed by drying at 80° C. under reduced pressure for about 18 hours. The dried solids (noroxymorphone, Solid 9, 17.9 g) were analyzed by HPLC as described above in Example 1.1. (Impurity 1=19 ppm; Impurity 2=1012 ppm).

Decolorization of crude noroxymorphone Batch 5 followed by hydrogenation improved operability, reduced the amount of Impurity 1, and visibly lightened the appearance of the solids from the starting material. The carbon treatment performed in multiple portions (Filtrate 6) or stressed for about 60 hours (Filtrate 9) at temperature followed by hydrogenation produced material that was comparable to the standard reaction conditions (Solid 3) with respect to Impurity 1 levels (see Table 14).

TABLE 14
Impurities in Batch 5 After Decolorization and Hydrogenation

| | Impurity 1 | Impurity 2 | |
|---|---|---|---|
| Sample | (ppm)^{a} | (ppm)^{a} | YI ^{b} |
| Solid 2 | 973 | 936 | 16.8 |
| Solid 3 | 20 | 662 | 14.2 |
| Solid 5 | 981 | 956 | — |
| Solid 6 | 22 | 566 | 9.7 |
| Solid 8 | 868 | 1019 | — |
| Solid 9 | 19 | 1012 | — |

^{a}Using HPLC method of Example 1.1., RSD is not more than ±20%.
^{b} Colorimetric concentrations were measured at about 4 mg/mL in 11.15% H₃PO₄.

### Example 3.2

### Batch 1 as the Noroxymorphone Source

Batch 1 was identified as the most colored batch (i.e., having the highest YI of 152.4 at a concentration of 4 mg/mL).

To a 500-mL jacketed reactor was charged water (300 mL) and 85% aqueous H₃PO₄ (835.3 mmol, 2.7 molar equivalents). The solution was heated to about 50° C. with agitation and noroxymorphone (304.5 mmol, Batch 1) was added in several portions. The resultant solution was not transparent. Activated carbon (Darco KB-G used “as is”, 30.0 g, 26 wt %) was charged in one bolus and the reaction mixture was heated to 90° C. The mixture was held at that temperature for about 6 hours. The hot reaction mixture was filtered through Whatman #1 filter paper and the carbon bed was rinsed with water (2×25 mL/rinse) to provide Filtrate 10 (569.8 g).

A 5.0 mL aliquot of Filtrate 10 was carried through the salt-breaking procedure as follows. To a 20 mL scintillation vial equipped with a magnetic stir bar was charged 5.0 mL of the noroxymorphone filtrate (Filtrate 10). Aqueous ammonium hydroxide (28-30 wt %) was added dropwise over 5 minutes. The pH after base addition was about 8. The solids were isolated by vacuum filtration and washed with water (2×25 mL/wash). The solids were dried at 80° C. under reduced pressure for about 16 hours. The dried solids (noroxymorphone, Solid 11, 4.0 g) were analyzed by HPLC as described above in Example 1.1. (Impurity 1=693 ppm; Impurity 2=375 ppm) and color (YI=11.5 at a concentration of 4 mg/mL).

The remaining portion of Filtrate 10 was hydrogenated using the procedure described in Example 3.1 The post hydrogenation filtrate (756.7 g) was charged into a 2-L round bottom flask and worked-up using the procedure described in Example 3.1. The dried solids (noroxymorphone, Solid 12, 76.7 g) were analyzed by HPLC as described above in Example 1.1. (Impurity 1=26 ppm; Impurity 2=794 ppm) and color (YI=15.5 at a concentration of 4 mg/mL).

### Example 3.3

### Batch 3 as the Noroxymorphone Source

Batch 3 was identified as the least colored batch (i.e., having the lowest YI of 116.6 at a concentration of 4 mg/mL). To a 500-mL jacketed reactor was charged water (348 mL), 85% aqueous H₃PO₄ (845.5 mmol, 2.4 molar equivalents), noroxymorphone (352.3 mmol, Batch 3) and activated carbon (Darco KB-G used “as is”, 30.1 g, 26 wt %). The mixture was heated to 90° C. with agitation and held at that temperature for about 6 hours. The hot reaction mixture was filtered and the carbon bed was rinsed with water (25 mL). The filtrate (Filtrate 13, 647.42 g) was hydrogenated and worked-up analogously to the procedure described in Example 3.1.

The wet cake was allowed to dry under suction for about 1 hour. The solids were dried at 80° C. under reduced pressure for about 16 hours. The dried solids (noroxymorphone, Solid 14, 94.0 g) were analyzed by HPLC as described above in Example 1.1. (Impurity 1=ND (i.e. <5 ppm’); Impurity 2=771 ppm) and color (YI=7.9 at a concentration of 4 mg/mL).

To a 500-mL flask was charged the already used activated carbon from above (about 30 g, water wet) and water (about 200 mL). The mixture was heated to 95° C. with agitation and held at that that temperature for about 10 minutes. The hot reaction mixture was filtered and the noroxymorphone solids were isolated by pH adjustment, at 75° C., with aqueous ammonium hydroxide (28-30 wt %). The final pH of the mixture was 8.2 using a calibrated (temperature corrected) pH meter. The mixture was cooled to a temperature of about 25° C. The solids were isolated by vacuum filtration and washed with water (2×25 mL/wash). The wet cake was allowed to dry under suction for about 1 hour followed by further drying at 80° C. under reduced pressure for about 16 hours. The dried solids (noroxymorphone, Filtrate 15, 5.4 g) were analyzed by HPLC as described above in Example 1.1. (Impurity 1=1227 ppm; Impurity 2=612 ppm) and color (YI=2.2 at a concentration of 4 mg/mL).

The results for the carbon treatment followed by hydrogenation are summarized below in Table 15.

TABLE 15
Impurities in Batches 1 and 3 After
Decolorization and Hydrogenation

| | Impurity 1 | Impurity 2 | |
|---|---|---|---|
| Sample | (ppm)^{a} | (ppm)^{a} | YI ^{b} |
| Batch 5 | 811 | 780 | 144.0 |
| Solid 3 | 20 | 662 | 14.2 |
| Batch 1 | 803 | 670 | 152.4 |
| Solid 12 | 26 | 794 | 15.5 |
| Batch 3 | 985 | 865 | 116.6 |
| Solid 14 | ND | 771 | 7.9 |

^{a}Using HPLC method of Example 1.1. ND—non detectable (<5 ppm)
^{b} Colorimetric concentrations were measured at about 4 mg/mL in 11.15% H₃PO₄.

### Example 4

### Hydrogenation Reactions of Noroxymorphone

The following is an example of the decolorization of noroxymorphone Batch 1 using activated carbon in order to prepare a stock “decolorized” filtrate for hydrogenation studies.

To a solution of noroxymorphone (0.344 mol, Batch 1) in water (375.5 g) and 85% aqueous H₃PO₄ (95.5 g, 2.4 molar equivalents) was added Darco KB-G (29.8 g). This mixture was heated to 90° C. with stirring for 16 hours. The mixture was then cooled to 60±5° C. and filtered. The filter cake was washed with water (3×86.7 g/wash) to yield 779.4 g of filtrate containing noroxymorphone. This procedure was repeated three more times and all filtrates from the four reactions were combined to give a stock solution of 12.8 wt % noroxymorphone in aqueous H₃PO₄ (3093.5 g).

### Example 4.1

### Hydrogenating Decolorized Batch 1

All hydrogenation reactions were carried out in the same manner A general description of the process used is as follows.

A sample of the above stock solution of decolorized Batch 1 (499.6 g, 64.0 g noroxymorphone) was charged to a pressure vessel followed by 5% palladium on carbon (2.0 g, 50% water wet, Johnson Matthey Type A101002-5). This mixture was then purged with nitrogen, heated to 80° C. and pressurized with hydrogen (515 kPa). The mixture was stirred under constant hydrogen pressure for 18 hours followed by venting and purging with nitrogen. A sample was analyzed by the HPLC method of Example 1.1. for levels of Impurity 1 and Impurity 2.

### Example 4.2

### Isolation of Purified Noroxymorphone

In order to determine the effects of varying the work-up conditions, noroxymorphone hydrogenated in Example 4.1. was worked-up and isolated under different conditions.

### Example 4.2.1

### Hydrogenation Sample 1—Polish Filtration Through Polypropylene; pH Adjustment 75° C.; Isolation at 25° C.

The post hydrogenation reaction mixture was filtered through a 5 μm polypropylene filter disc twice and the filter cake was washed with water (2×78 g/wash). The filtered mixture was then transferred to a 0.5 L jacketed reactor. The batch was heated to 75° C. (pH=1.6) and aqueous ammonium hydroxide (28-30 wt %, 95 g) was added over 30 minutes to give a final pH of 8.6. The mixture was cooled to 25° C. (pH=9.8) and the solids were isolated by filtration. The wet cake was washed with water (2×78 g/wash) and conditioned under vacuum suction for about 1 hour. The solids were then dried at 80° C. under reduced pressure for 16 hours.

### Example 4.2.2

### Hydrogenation Sample 2—pH Adjustment 75° C.; Isolation at 60° C.

The post hydrogenation reaction mixture was filtered through a 5 μm polypropylene filter disc twice and the filter cake was washed with water (2×85 g/wash). The filtered mixture was then transferred to a 0.5 L jacketed reactor. The batch was heated to 75° C. (pH=1.6) and aqueous ammonium hydroxide (28-30 wt %, 90 g) was added over 30 minutes to give a final pH of 8.2. The mixture was cooled to 60±5° C. and the solids were isolated by filtration. The wet cake was washed with water (2×85 g/wash) and conditioned under vacuum suction for about 1 hour. The solids were then dried at 80° C. under reduced pressure for 16 hours.

### Example 4.2.3

### Hydrogenation Sample 3—Polish Filtration Through Nylon; pH Adjustment 75° C.; Isolation at 25° C.

The post hydrogenation reaction mixture was filtered through filter paper and the filter cake was washed with water (2×78 g/wash). The resultant filtrate was filtered through a Nylon 0.45 μm filter disc. The filtered mixture was then transferred to a 0.5 L jacketed reactor. The batch was heated to 75° C. (pH=1.4) and aqueous ammonium hydroxide (28-30 wt %, 83 g) was added over 30 minutes to give a final pH of 8.6. After 10 minutes at 75±5° C., the mixture was cooled to 25±5° C. and the solids were isolated by filtration. The wet cake was washed with water (2×78 g/wash) and conditioned under vacuum suction for about 1 hour. The solids were then dried at 25° C. under reduced pressure for 16 hours.

### Example 4.2.4

### Hydrogenation Sample 4—pH Adjustment 75° C.; Isolation at 75° C.

The post hydrogenation reaction mixture was filtered through a 5 μm polypropylene filter disc and the filter cake was washed with water (2×78 g/wash). The filtered mixture was then transferred to a 0.5 L jacketed reactor. The batch was heated to 75° C. (pH=1.6) and aqueous ammonium hydroxide (28-30 wt %, 90 g) was added over 20 minutes to give a final pH of 8.4. The solids were isolated at 75° C. by filtration. The wet cake was washed with water (2×78 g/wash) and conditioned under vacuum suction for about 1 hour. The solids were then dried at 80° C. under reduced pressure for 16 hours.

### Example 4.2.5

### Hydrogenation Sample 5—Partial pH Adjustment to pH 5 at 25° C.; Final pH Adjustment to pH 8.5 at 75° C.; Isolation at 25° C.

The post hydrogenation reaction mixture was filtered through filter paper and the filter cake was washed with water (2×78 g/wash). The resultant filtrate was filtered through a Nylon 0.45 μm filter disc. The filtered mixture was then transferred to a 0.5 L jacketed reactor. The batch was adjusted to 25±5° C. (pH =1.8) and aqueous ammonium hydroxide (28-30 wt %) was added to achieve a pH of 5.0, keeping the temperature below 30° C. The batch was then heated to 75° C. and the remaining ammonium hydroxide (90 g total) was added to give a final pH of 8.5 at 75° C. After 10 minutes at 75±5° C., the mixture was cooled to 25±5° C. and the solids were isolated by filtration. The wet cake was washed with water (2×78 g/wash) and conditioned under vacuum suction for about 1 hour. The solids were then dried at 80° C. under reduced pressure for 16 hours.

### Example 4.3

### Results

In Table 16 below is a tabulated summary of results from the scale-up purification runs conducted above.

TABLE 16
Impurities after Hydrogenation Reaction

| | | Isolated Solid (ppm)^{a} | | |
|---|---|---|---|---|
| | | Impurity 1 | Impurity 2 | Yield, % |
| Batch 1 | Batch 1 ^{b} | 803 | 670 | N/A |
| | Hydrogenation Sample 1 | 21 | 1202 | 92.3 |
| | (Example 4.2.1) | | | |
| | Hydrogenation Sample 2 | 19 | 1184 | 92.3 |
| | (Example 4.2.2) | | | |
| | Hydrogenation Sample 3 | <5 | 1180 | 87.6 |
| | (Example 4.2.3) | | | |
| | Hydrogenation Sample 4 | <5 | 1144 | 92.3 |
| | (Example 4.2.4) | | | |
| | Hydrogenation Sample 5 | 18 | 1221 | 94.1 |
| | (Example 4.2.5) | | | |

^{a}Using HPLC method of Example 1.1.
^{b} N/A = not analyzed.

All solids isolated above yielded noroxymorphone with levels of 14-hydroxymorphinone within no more than 40 ppm. All lots of noroxymorphone had a similar appearance (i.e., of a beige solid).

### Example 5

### Larger Scale Preparations of Noroxymorphone

The following example describes larger-scale preparations of noroxymorphone having a low level of ABUK, e.g., Impurity 1.

### Example 5.1

### Carbon Treatment of Batch 6 and Batch 8

Below is an example decolorization of noroxymorphone originating from Example 2 Batches 6 and 8 using activated carbon in order to prepare a stock “decolorized” filtrate for hydrogenation studies.

To a solution of noroxymorphone (0.347 mol, Batch 6) in water (372.7 g) and 85% aqueous H₃PO₄ (95.9 g, 2.4 molar equivalents) was added Darco KB-G (used “as is”, 29.9 g, 26 wt %). This mixture was heated to 90° C. with stirring for 16 hours. The mixture was then cooled to 60±5° C. and filtered. The filter cake was washed with water (3×86.0 g/wash) to yield 793.3 g of filtrate containing noroxymorphone. This procedure was repeated three more times using noroxymorphone from either Batch 6 (once) or Batch 8 (twice), and all filtrates from the four reactions were combined to give a stock solution of noroxymorphone in aqueous H₃PO₄ (3090.8 g, 12.9 wt % noroxymorphone). This solution was then used as described below.

### Example 5.2

### Hydrogenation and Isolation of Decolorized Batch 6 and Batch 8

A sample of stock solution (503.7 g, 64.9 g noroxymorphone) was charged to a pressure vessel followed by 5% palladium on carbon (2.1 g, 50% water wet, Johnson Matthey Type A101002-5). This mixture was then purged with nitrogen, heated to 80° C. and pressurized with hydrogen (517 kPa). The mixture was stirred under constant hydrogen pressure for 18 hours. An in-process-control sample (715 μL) was filtered through a 0.45 μm nylon syringe filter, diluted to 25 mL with 0.85% aqueous H₃PO₄, and analyzed by HPLC as described above in Example 1.1. for levels of Impurity 1 and Impurity 2. The remainder of the mixture was filtered and the filter cake was washed with water (2×85 g/wash). The filtrate was then filtered through a nylon 0.45 μm membrane. The polished filtrate was then transferred to a 0.5 L jacketed reactor. The batch was adjusted to 25±5° C. (pH=1.6 corrected for temperature) and aqueous ammonium hydroxide (28-30 wt %) was added to achieve a pH of 5.0 (corrected for temperature) keeping the temperature below 30° C. The batch was then heated to 75±5° C. (pH=4.7 corrected for temperature) and the remaining ammonium hydroxide (89 g total) was added to give a final pH of 8.1 at 75° C. (corrected for temperature). The batch was then cooled to 25±5° C. (pH=9.5 corrected for temperature) and the solids were isolated by filtration. The wet cake was washed with water (2×85 g/wash) and conditioned under vacuum suction for about 1 hour. The solids were then dried at 80° C. under reduced pressure for 16 hours. Impurity 1 and Impurity 2 levels in the noroxymorphone product were determined as described in Example 1.1. The experiment was run 5 times.

### Example 5.3

### Results

In Table 17 below is a tabulated summary of results from the scale-up purification run conducted above.

TABLE 17
Summary of Results for Processing Batch 6 and Batch 8

| | | Impurity Amount (ppm)^{a} | |
|---|---|---|---|
| | | Impurity 1 | Impurity 2 |
| Batch 6 | Average of Batches (6 + 8) | 884 | 759 |
| & | Run 1 | 17 | 1052 |
| Batch 8 | Run 2 | 18 | 1088 |
| (50:50) | Run 3 | 18 | 1077 |
| | Run 4 | ND | 892 |
| | Run 5 | ND | 541 |

^{a}Using HPLC method of Example 1.1.

### Example 5.4

### Decolorizing and Hydrogenating Batches 5 and 6

Below is an example decolorization of noroxymorphone originating from Example 2 Batches 5 and 6 using activated carbon followed by hydrogenation.

Decolorization of noroxymorphone originating from Example 2 Batches 5 and 6 using activated carbon to prepare a decolorized filtrate was conducted under similar conditions described in Example 5.1. above; however, the quantities of materials used in this example were further scaled-up except that the amount of water used was not scaled-up proportionally, that is, the solution containing noroxymorphone and 85% aqueous H₃PO₄ was more concentrated than in Example 5.1. A sample of the decolorized filtrate was analyzed according to the HPLC method of Example 1.1.; FIG. 1A shows the resulting HPLC chromatogram obtained. Peak base-lines are indicated by dotted lines; peak base-line end-points are indicated by triangles. The peak at 11.663 minutes denotes the presence of 14-hydroxynormorphinone (Impurity 1). To make the peak at 11.663 minutes more readily visible, FIG. 1B shows an about 115 times enlargement of the 1-15 minute portion of the HPLC chromatogram of FIG. 1A. The decolorized filtrate was used as the starting material for hydrogenation according to the process of the disclosure under similar conditions described in Example 5.2. above; however, as previously mentioned the quantities of materials used in this example were further scaled-up. A sample of the noroxymorphone produced was analyzed according to the HPLC method of Example 1.1.; FIG. 2A shows the resulting HPLC chromatogram obtained. Again, peak base-lines are indicated by dotted lines; peak base-line end-points are indicated by triangles. Notably, a peak at about 11.70 minutes, corresponding to 14-hydroxynormorphinone (Impurity 1), was absent in the FIG. 2A chromatogram—compare the FIG. 2A chromatogram against the chromatogram of the starting material shown in Figure IA. To make this region of the FIG. 2A chromatogram more readily visible, FIG. 2B shows an about 106 times enlargement of the 1-15 minute portion of the HPLC chromatogram of FIG. 2A. Notably, even after enlargement a peak at about 11.70 minutes remains absent in FIG. 2B—compare FIG. 2B against the enlarged chromatogram of the starting material shown in FIG. 1B.

### Example 6

### Methods for Suppressing Impurity 3 Formation Relative to Noroxymorphone

A variety of methods for suppressing the formation of Impurity 3 relative to noroxymorphone were evaluated and are described below.

### Example 6.1

### Varying the Catalyst

In order to evaluate the occurrence of ring-opening and the formation of impurity 3,4,14-trihydroxymorphinan-6-one (Impurity 3) during the hydrogenating of noroxymorphone, experiments were conducted with different catalysts in representative purification processes. It was noted that the Impurity 3 peak has an RRT of 0.78 when compared to the noroxymorphone peak, each determined according to the HPLC method of Example 1.2.

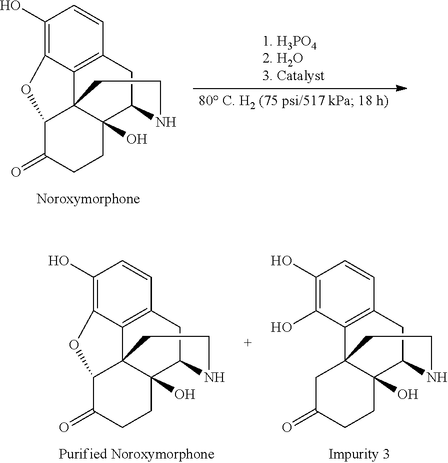 [SMILES: O=C1CC[C@@]2(O)[C@H]3Cc4ccc(O)c5c4[C@@]2(CCN3)[C@H]1O5 ; O=C1CC[C@@]2(O)[C@H]3Cc4ccc(O)c5c4[C@@]2(CCN3)[C@H]1O5 ; O=C1CC[C@@]2(O)[C@H]3Cc4ccc(O)c(O)c4[C@@]2(CCN3)C1]

Individual reaction vials (8 mL) were charged with stock solution (5 mL, 570 mg noroxymorphone) followed by one of the catalysts (7.5 wt %, about 43 mg each) as described below in Table 18. The reactions were then run under identical hydrogenation conditions (80° C., 517 kPa H₂, 18 h) on a Parallel Hydrogenation apparatus. Samples of each reaction mixture (215 μL) were diluted to 20 mL with 0.085% aqueous H₃PO₄ and analyzed by HPLC as described above in Example 1.2. where the area under the peaks of Impurity 3 and noroxymorphone were determined. The ratio of noroxymorphone: Impurity was calculated as follows:

Impurity 3%=(Impurity 3 peak area)×100/[Impurity 3 peak area+noroxymorphone peak area].

Noroxymorphone %=(Noroxymorphone peak area)×100/[Impurity 3 peak area+noroxymorphone peak area].

The results are summarized below in Table 18.

TABLE 18
Palladium on Carbon Catalysts vs. Formation of Impurity 3

| | | Noroxymorphone:Impurity 3 |
|---|---|---|
| Catalyst (Type) | Supplier | (Peak Area %) |
| 5% Pd/C | Johnson Matthey | 88:12 to 93:7 ^{a} |
| (A101002-5) | | |
| 5% Pd/C | BASF | 59:41 |
| (ESCAT ™ 147) | | |
| 5% Pd/C | BASF | 64:36 |
| (ESCAT ™ 143) | | |
| 10% Pd/C | Evonik | 57:43 |
| (E101 NE/W) | | |
| 5% Pd/C | Johnson Matthey | 73:27 |
| (5R39) | | |
| 5% Pd/C | Johnson Matthey | 53:47 |
| (A405028-5) | | |
| 5% Pd/C | Johnson Matthey | 87:13 |
| (A503023-5) | | |
| 5% Pd/C | BASF | 16:84 |
| (CP-97 EUW) | | |
| 5% Pd/C | BASF | 41:59 |
| (CP-86 EUW) | | |
| 5% Pd/C | BASF | 8:92 |
| (CP-126 EUW) | | |
| 5% Pd(S)/C | Johnson Matthey | 98.6:1.4 |
| (A103038-5) | | |
| 5% Pd/BaSO₄ | Johnson Matthey | 99.8:0.2 |
| (A308053-5) | | |
| 5% Pd/BaSO₄ | Johnson Matthey | 99.1:0.8 |
| (A201053-5) | | |

^{a} Ranges observed over the course of multiple (>5) experiments.

All of the carbon-based catalysts tested provided higher levels of Impurity 3 versus the catalyst Johnson Matthey Type A101002-5 when charged at identical weight % (7.5 wt % vs. noroxymorphone). A catalyst, poisoned with sulfur (5% Pd(S)/C, Type A103038-5), provided only 1.4% of Impurity 3 versus the best Pd/C-based catalyst, Type A101002-5, which provided 7%-12% of Impurity 3. Both palladium catalysts supported on barium sulfate (BaSO₄) yielded less than 1% of Impurity 3.

### Example 6.2

### Addition of Sodium Iodide

In order to test the activity of sodium iodide levels on the suppression of Impurity 3 formation during the hydrogenating of noroxymorphone, experiments were conducted in parallel, where sodium iodide levels in representative purification processes were sequentially lowered. Two control experiments were also run in which no sodium iodide was added.

### Example 6.2.1

### Generation of Noroxymorphone Stock Solution

A solution of noroxymorphone was prepared by dissolving purified noroxymorphone (26.05 g) in water (181.0 g) and 85% aqueous H₃PO₄ (21.4 g). This yielded a final noroxymorphone concentration of about 114 mg/mL.

### Example 6.2.2

### Generation of Sodium Iodide Solutions

An aqueous sodium iodide solution (10 mg/mL) was prepared by dissolving 250 mg of sodium iodide in 25 mL of water. Samples of this solution were diluted to 1 mg/mL and 0.1 mg/mL by serial dilutions. Aliquots of these solutions were then dosed into individual reaction to achieve the desired levels of sodium iodide identified in Table 19.

### Example 6.2.3

### Hydrogenation

Individual reaction vials (8 mL) were charged with a noroxymorphone stock solution (5 mL, 570 mg noroxymorphone) followed by 5% palladium on carbon (43 mg, 7.5 wt %, Johnson Matthey Type A101002-5). To these mixtures were added sodium iodide solutions of varying concentration. Hydrogenation reactions were then run under identical reducing conditions (80° C., 517 kPa H₂, 18 h) on a Parallel Hydrogenation apparatus. Samples of each reaction mixture (215 μL) were diluted to 20 mL with 0.085% aqueous H₃PO₄ then analyzed by HPLC as described above in Example 1.2. where the area under the peaks of Impurity 3 and noroxymorphone were determined. The Impurity 3 peak area % and the noroxymorphone peak area % were determined as described in Example 6.1. above. The results are summarized below in Table 19.

TABLE 19
Levels of Impurity 3 Generated vs. NaI Loading

| Sodium Iodide | Noroxymorphone:Impurity 3 |
|---|---|
| (ppm)^{a} | (Peak Area %) |
| 1000 | 100:0 ^{b} |
| 750 | 100:0 ^{b} |
| 500 | 100:0 ^{b} |
| 250 | 100:0 ^{b} |
| 200 | 99.8:0.2 |
| 100 | 98.8:1.2 |
| 50 | 97.4:2.6 |
| 25 | 94.5:5.5 |
| 10 | 93.0:7.0 |
| 5 | 88.3:11.7 |
| 0 | 87.0:13.0 |
| 0 | 89.0:11.0 |

^{a}Based on about 570 mg of noroxymorphone charged per reaction.
^{b} Not detected (<0.01%).

For this screening study, only levels of noroxymorphone versus the ring-opened 3,4,14-trihydroxymorphinan-6-one (Impurity 3) were compared. Sodium iodide levels of at or above 200 ppm were shown to suppress formation of Impurity 3 to levels≦0.2%. The effect of sodium iodide on levels of ring-opening were evident at sodium iodide loadings of at or above 10 ppm, albeit to a lesser extent at below 200 ppm. As can be noted from the results presented in Table 19, very low levels of sodium iodide (5 ppm) seemed to have no significant effect on suppression of Impurity 3 formation.

### Example 6.3

### Ring Opening in the Presence of Chloride

Four separate pressure vessels were charged with purified noroxymorphone (25.00 g), water (122 g) and 85% aqueous H₃PO₄ (20.66 g). To three of the reaction mixtures was added either ammonium chloride (2.73 g, 0.05 mol, sodium chloride (2.98 g, 0.05 mol) or 37% hydrochloric acid (4.64 mL, 0.05 mol). The fourth vessel was used as a control with no additional chloride added. A 5% palladium on carbon catalyst (1.88 g, 7.5 wt %, Johnson Matthey Type A101002-5) was then added to each vessel and the mixtures were heated to and kept at 80° C. under a hydrogen pressure of 517 kPa for 18 hours. The hydrogenation reaction products were cooled to a temperature of about 25° C., and a sample was removed by syringe and filtered using a 0.45 μm filter cartridge. A sample of the filtrate (215 μL) was diluted to 20 mL with 0.085% aqueous H₃PO₄ then analyzed by HPLC as described above in Example 1.2. where the area under the peaks of Impurity 3 and noroxymorphone were determined. The Impurity 3 peak area % and the noroxymorphone peak area % were determined as described in Example 6.1. above except that the total area of all of the observed HPLC peaks was used in place of [Impurity 3 peak area+noroxymorphone peak area]. The results are summarized below in Table 20.

TABLE 20
Effect of Chloride Anion on Suppression of Impurity 3

| | |
|---|---|
| Compound or Impurity | Added Chloride |

| (Peak Area %) | None | NH₄Cl | NaCl | 37% HCl |
|---|---|---|---|---|
| Noroxymorphone | 89.64 | 98.80 | 98.81 | 98.79 |
| Impurity 3 | 7.33 | 0.06 | 0.06 | 0.05 |

As shown in Table 20, addition of equimolar amounts of chloride seemed to have a nearly identical effect on the suppression of the formation of Impurity 3 and the effect was independent of the source of chloride (e.g., NH₄Cl, NaCl or HCl).

### Example 6.4

### Ring-Opening in the Presence of Different Amounts of Sodium Chloride

Separate pressure vessels were charged with purified noroxymorphone (25.00 g), water (122 g) and 85% aqueous H₃PO₄ (20.66 g). Sodium chloride was then added to each vessel at 1.0, 2.5, 5.0, 7.5 or 11.9 weight % based on the noroxymorphone charged. A control with no added sodium chloride was also run. A 5% palladium on carbon catalyst (1.88 g, 7.5 wt %, Johnson Matthey Type A101002-5) was then added to each vessel and the mixtures were heated to and kept at 80° C. under a hydrogen pressure of 517 kPa for 18 hours. The hydrogenation reaction products were cooled to a temperature of about 25° C., and a sample was removed by syringe and filtered using a 0.45 μm filter cartridge. A sample of the filtrate (215 μL) was diluted to 20 mL with 0.085% aqueous H₃PO₄ then analyzed by HPLC analysis as described above in Example 1.2. where the area under the peaks of Impurity 3 and noroxymorphone were determined. The Impurity 3 peak area % and the noroxymorphone peak area % were determined as described in Example 6.1. above except that the total area of all of the observed HPLC peaks was used in place of [Impurity 3 peak area+noroxymorphone peak area]. The results are summarized below in Table 21.

TABLE 21
Effect of Chloride Anion Concentration
on Suppression of Impurity 3

| Compound or Impurity | wt. % NaCl | | | | | |
|---|---|---|---|---|---|---|
| (Peak Area %) | 0 | 1.0 | 2.5 | 5.0 | 7.5 | 11.9 |
| Noroxymorphone | 89.64 | 97.52 | 98.41 | 98.72 | 98.75 | 98.81 |
| Impurity 3 | 7.33 | 0.78 | 0.27 | 0.10 | 0.10 | 0.06 |

As shown in Table 21, the addition of NaCl suppresses the formation of Impurity 3. In comparison to iodide (see results using sodium iodide presented in Table 19 above), chloride is less effective in suppressing Impurity 3 and larger quantities of chloride are required to obtain comparable results.

### Example 7

### Conversion of Noroxymorphone to Naloxone

Noroxymorphone containing about 15 ppm of 14-hydroxymorphinone was converted to naloxone in 70% yield (98.1% by weight) containing about 6 ppm 7,8-didehydronaloxone (Impurity 4) as summarized in Scheme 13 below.

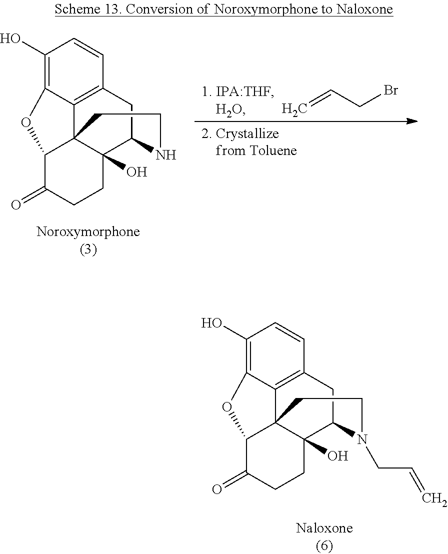

A 500 mL jacketed reactor was charged with noroxymorphone containing 15 ppm 14-hydroxymorphinone (50.00 g, 0.174 mol, 1.0 equiv), sodium bicarbonate (23.39 g, 0.278 mol, 1.6 equiv.) and iso-propanol/tetrahydrofuran (60 IPA:40 THF v/v, 325.4 mL). The water content of the mixture was adjusted to 30 wt % with respect to the noroxymorphone charge by adding deionized water (about 15.2 mL). Allyl bromide (29.48 g, 0.243 mol, 1.4 equiv.) was added and the mixture was heated to and kept at 63±2° C. with stirring for a minimum of 10 h. The mixture was cooled to 5±5° C. and then the solids were removed by filtration. The filtrate was diluted with tetrahydrofuran (224.0 mL) and the mixture was transferred slowly to a boiling mixture of toluene (380.0 mL), water (92.0 mL) and sodium chloride (3.22 g). The solvents (iso-propyl alcohol and tetrahydrofuran) were removed by constant distillation and the liquid volume in the vessel was kept constant by addition of toluene. Upon completion of the filtrate transfer, the mixture temperature was adjusted to 80±5° C. while maintaining a continuous distillation.

A solution of sodium chloride was added (1.0 M, 14.2 mL) and, after complete mixing, agitation was stopped and the residual aqueous layer was removed from the vessel by the bottom outlet valve (BOV). A fresh portion of sodium chloride solution (1.0 M, 28.5 mL) was added and the temperature of the mixture was adjusted to 80° C. Agitation was stopped and again the aqueous layer was removed from the vessel by the BOV. The remaining organic layer was heated to 110±2° C. while maintaining continuous distillation again keeping the initial volume constant by the addition of toluene. The mixture was then filtered hot (above 90° C.) and the resultant filtrate was transferred into a jacketed reactor. The mixture was then cooled over 5 hours to 60° C., and heptane (342.0 mL) was slowly added maintaining a temperature of about 60° C. The batch was held at 60° C. for 4 hours and then cooled to 10° C. at a rate of 8° C./min. The solids were filtered and dried to constant weight in a reduced pressure oven at 80° C. This yielded naloxone as a light beige solid (40.84 g, 70% yield). This material contained about 6 ppm Impurity 4 upon HPLC analysis as described in Example 1.3.

### Example 8

### Conversion of Naloxone to Naloxone Hydrochloride

For this experiment, naloxone containing about 7 ppm of 7,8-didehydronaloxone (Impurity 4) was converted to naloxone hydrochloride in 75% yield (99.9% by weight) containing about 8 ppm Impurity 4 as summarized in Scheme 14 below.

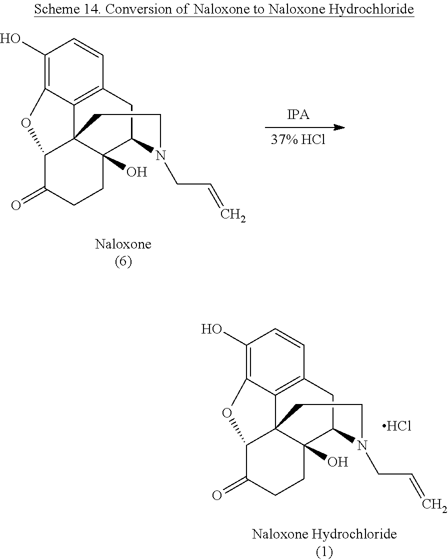 [SMILES: C=CCN1CC[C@]23c4c5ccc(O)c4O[C@H]2C(=O)CC[C@@]3(O)[C@H]1C5 ; C=CCN1CC[C@]23c4c5ccc(O)c4O[C@H]2C(=O)CC[C@@]3(O)[C@H]1C5 ; Cl]

### Example 8.1

### Initial Decolorization of Naloxone

A jacketed reactor was charged with naloxone containing about 7 ppm Impurity 4 (15.35 g) and iso-propanol (50.54 mL). This mixture was heated under nitrogen to reflux for 1 hour and then cooled to about 25° C. A mixture of butylated hydroxytoluene (0.15 g, 1 wt %) and activated carbon (Darco KB-WJ, 1.50 g, 10 wt %) in iso-propanol (11.72 mL) was then added. The reaction mixture was purged with nitrogen (14-21 kPa) for 10 minutes, heated to 75° C. and held at 75° C. for 1 hour while maintaining the nitrogen purge (14-21 kPa). This mixture was pressure filtered at 80° C. through a 1.2 μm polypropylene filter. A mixture of butylated hydroxytoluene (0.15 g, 1 wt %) and activated carbon (Darco KB-WJ, 1.51 g, 10 wt %) in iso-propanol (10.4 mL) was added to the filtrate and the above decolorization process was repeated. The carbon cake was washed with 11.4 mL of iso-propanol and the resultant filtrate was used directly in Example 8.2. as described below.

### Example 8.2

### Salt Formation and Isolation

Degassed water (13.1 mL) was added to the resultant filtrate from Example 8.1. above and the mixture was purged with nitrogen (14-21 kPa) for 10 minutes and then heated to 75° C. while maintaining the nitrogen purge (14-21 kPa). Hydrochloric acid (37%, 1.10 equiv.) was slowly added to the mixture keeping the temperature below 80° C. Upon complete addition, the HCl addition line was rinsed with 3.5 mL of degassed water, the mixture was cooled to 67° C. and naloxone hydrochloride dihydrate seed crystals (0.12 g) were charged to the mixture. The batch was then cooled to 55° C. at a rate of 2° C./h, held at this temperature for 5 hours and then further cooled over 8 hours to −10° C. The solids were isolated by filtration and the resultant wet cake was washed with iso-propanol:water (85:15, 12° C., 15.0 mL). The solids were then dried to constant weight at a temperature of about 25° C. under reduced pressure. This yielded naloxone hydrochloride as a white crystalline solid (14.0 g, 75% yield) in 99.9% purity, as determined by HPLC, containing 8 ppm 7,8-didehydronaloxone hydrochloride, determined by HPLC as described above in Example 1.3.

### Example 9

### Conversion of Noroxymorphone to Naltrexone

Noroxymorphone was converted to naltrexone as summarized in Scheme 15 below.

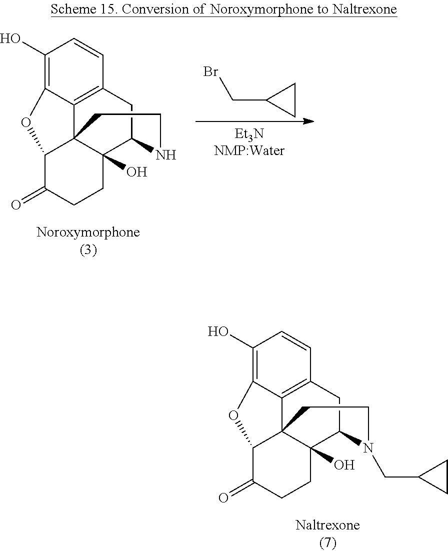 [SMILES: O=C1CC[C@@]2(O)[C@H]3Cc4ccc(O)c5c4[C@@]2(CCN3)[C@H]1O5 ; BrCC1CC1 ; O=C1CC[C@@]2(O)[C@H]3Cc4ccc(O)c5c4[C@@]2(CCN3CC2CC2)[C@H]1O5]

Two experiments were conducted: one using purified noroxymorphone, purified as described above, as the starting material and the second using noroxymorphone starting material to which Impurity 1 was deliberately added. Each experiment is described below.

### Example 9.1

### Naltrexone Prepared from Purified Noroxymorphone

Cyclopropylmethyl bromide (5.29 g, 39.2 mmol, Minakem LLC, Hackensack, N.J.) and triethylamine (3.63 g, 35.9 mmol, Fisher Scientific, Pittsburgh, Pa.) were added to a suspension of purified noroxymorphone (11.01 g, 38.3 mmol, Rhodes Technologies, Coventry, R.I.) in a 10:1 mixture of N-methyl-2-pyrrolidone:water (vol.:vol., 36.3 mL) in a 250 mL reaction vessel. The purified noroxymorphone, purified as described in one of the examples above, contained <10 ppm Impurity 1. The vessel was purged with nitrogen and the reaction mixture was heated to 70° C. and kept at that temperature for 2 hours. Then, additional triethylamine (3.63 g, 35.9 mmol) was added. After 1 more hour at that temperature, additional cyclopropylmethyl bromide (1.11 g, 8.3 mmol) was added and the mixture was stirred for 2 hours more at 70° C. A sample of the reaction mixture was taken for HPLC analysis that indicated about 1.1% of the noroxymorphone starting material remained. Maintaining the mixture at about 70° C., water (165 mL) was then added drop-wise. This resulted in the formation of an oil which formed a gum upon cooling to a temperature of about 25° C. The reaction liquors were decanted off and the gum was dissolved in acetonitrile (22 mL). Water (100 mL) was added and the mixture was extracted with dichloromethane (2×75 mL/extraction). The combined extracts were dried (Na₂SO₄), filtered, and concentrated to dryness to provide 7.29 g of naltrexone as a light brown solid (56% yield).

Analysis was performed by HPLC as described in Example 1.4. The levels of naltrexone (95.1 area %, 89.5 wt. %) and 3-cyclopropylmethyl naltrexone (2.5 wt. %) in the reaction product were determined. The weight percent assay was below the expected value of about 100% probably because of the presence of residual N-methyl-2-pyrrolidone solvent. The HPLC analysis also determined that the reaction product contained about 18 ppm of the ABUK 7,8-didehydronaltrexone (designated as “Impurity 6”).

### Example 9.2

### Naltrexone Prepared from Noroxymorphone Containing Added Impurity 1

As a check on the above experimental procedure and analysis, Example 9.1. was repeated under substantially identical conditions except Impurity 1 (25 mg, 0.088 mmol, Rhodes Technologies; this corresponds to a level of about 2270 ppm of Impurity 1) was deliberately added to and present with the starting purified noroxymorphone. The sample of the reaction mixture taken for HPLC analysis indicated that about 1.3% of the noroxymorphone starting material remained. After the combined extracts were dried (Na₂SO₄), filtered, and concentrated to dryness, 7.95 g of naltrexone was obtained as a light brown solid (61% yield). Analysis by HPLC as described in Example 1.4. determined the levels of naltrexone (95.2 area %, 82.0 wt. %) and 3-cyclopropylmethyl naltrexone (2.2 wt. %) in the reaction product. As above, the weight percent assay was below the expected value of about 100% probably because of the presence of residual N-methyl-2-pyrrolidone solvent. The HPLC analysis also determined that the reaction product contained 1821 ppm of the Impurity 6 ABUK.

The invention is not to be limited in scope by the specific embodiments disclosed in the examples that are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims. A number of references have been cited, the entire disclosures of which are incorporated herein by reference for all purposes.

What is claimed:

1. A process for reducing the amount present of a compound of formula (I) or a salt or a solvate thereof: 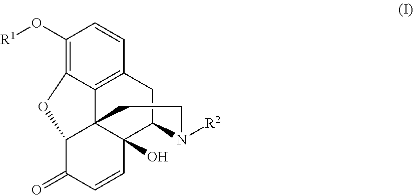
in a composition comprising a compound of formula (I) or a salt or a solvate thereof and a compound of formula (II) or a salt or a solvate thereof, wherein the compound of formula (II) is: 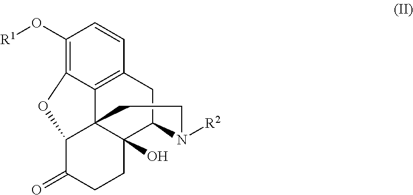
the process comprising:
(b) hydrogenating the compound of formula (I);
wherein:
R¹ is —H, (C₁-C₇)alkyl or an O-protecting group; and
R² is —H, —(C₂-C₄)alkenyl, —(C₂-C₇)alkyl, —(C₁-C₇)alkyl-(C₃-C₇)cycloalkyl, —CN, —C(═O)O—(C₁-C₆)alkyl, —C(═O)O-phenyl or a N-protecting group.

2. The process of claim 1, wherein le is —H.

3. The process of claim 1, wherein R¹ is an O-protecting group selected from the group consisting of acetate, ethyloxycarbonyl, pivolate, benzoate, tert-butyldiphenylsilyl, trimethylsilyl, triethylsilyl, tert-butyldimethylsilyl, benzyl, triphenylmethyl and tert-butyl.

4. The process of any one of claims 1 to 3, wherein R² is —H.

5. The process of any one of claims 1 to 3, wherein R² is —(C₂-C₄)alkenyl or —(C₁-C₇)alkyl-(C₃-C₇)cycloalkyl, and preferably is —CH₂CH═CH₂ or —CH₂-cyclopropyl.

6. The process of any of claims 1 to 3, wherein R² is a N-protecting group selected from the group consisting of acetamide, ethyloxycarbonyl, tert-butyloxycarbonyl, carbobenzyloxy, 9-fluorenylmethyloxycarbonyl, allyloxycarbonyl, tosyl, benzenesulfonyl, trifluoromethylcarbonyl, and 2,2,2-trichloroethoxycarbonyl.

7. The process of any one of claims 1, 2, and 4, wherein the compound of formula (I) is: 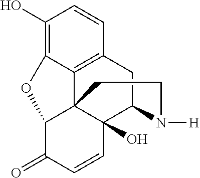 [SMILES: O=C1C=C[C@@]2(O)[C@H]3Cc4ccc(O)c5c4[C@@]2(CCN3)[C@H]1O5]
or a salt thereof,
and the compound of formula (II) is: 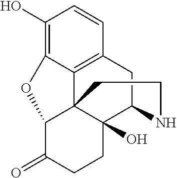 [SMILES: O=C1CC[C@@]2(O)[C@H]3Cc4ccc(O)c5c4[C@@]2(CCN3)[C@H]1O5]
or a salt thereof.

8. The process of any one of claims 1 to 6, wherein the compound of formula (I) is a salt shown as formula (Ia): 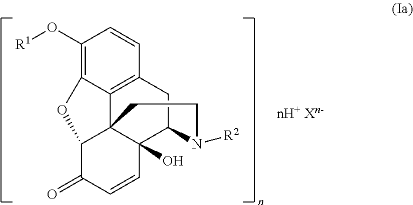
or a solvate thereof;
wherein:
R¹ and R² are defined as in any one of claims 1 to 6;
Xⁿ⁻ is an anion selected from the group consisting of Br⁻, succinate, tartrate, maleate, fumarate, citrate, NO₃⁻, Cl⁻, HSO₄⁻, SO₄²⁻, methanesulfonate, tosylate, trifluoroacetate, H₂PO₄⁻, HPO₄²⁻, [(NH₄)HPO₄]⁻, oxalate, perchlorate, H₃CC(O)O⁻, HC(O)O⁻, and mixtures thereof; and
n is 1 or 2.

9. The process of any one of claims 1 to 6 and 8, wherein the compound of formula (II) is a salt shown as formula (IIa): 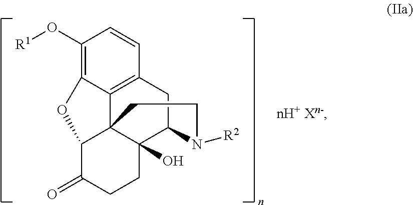
or a solvate thereof;
wherein:
R¹ and R² are defined as in any one of claims 1 to 6;
Xⁿ⁻ is an anion selected from the group consisting of Br⁻, succinate, tartrate, maleate, fumarate, citrate, NO₃⁻, Cl⁻, HSO₄⁻, SO₄²⁻, methanesulfonate, tosylate, trifluoroacetate, H₂PO₄⁻, HPO₄², [(NH₄)HPO₄]⁻, oxalate, perchlorate, H₃CC(O)O⁻, HC(O)O⁻, and mixtures thereof; and
n is 1 or 2.

10. The process of claim 8 or 9, wherein n is 1 or 2, and preferably n is 1.

11. The process of any one of claims 8 to 10, wherein Xⁿ⁻ is selected from the group consisting of HSO₄⁻, SO₄²⁻, H₂PO₄²⁻, HPO₄²⁻, H₃CC(O)O⁻, HC(O)O⁻, and mixtures thereof.

12. The process of any one of claims 8 to 10, wherein Xⁿ⁻ is selected from the group consisting of H₂PO₄⁻, HPO₄²⁻, [(NH₄)HPO₄]⁻, H₃CC(O)O⁻, HC(O)O⁻, and mixtures thereof.

13. The process of any one of claims 8 to 10 and 12, wherein Xⁿ⁻ is selected from the group consisting of H₂PO₄⁻, HPO₄²⁻, [(NH₄)HPO₄]⁻, and mixtures thereof.

14. The process of any one of claims 8 to 13, wherein the compound of formula (Ia), the compound of formula (IIa), or the compounds of formulae (Ia) and (IIa) is obtained by adding an acid H⁺ₙ Xⁿ⁻ to the reaction composition before, during, or before and during the hydrogenation reaction of step (b).

15. The process of claim 14, wherein the acid H⁺ₙXⁿ⁻ is selected from the group consisting of H₂SO₄, H₃PO₄, HC(O)OH, and CH₃C(O)OH, and preferably is H₃PO₄.

16. The process of claim 14 or 15, wherein the acid H⁺ₙXⁿ⁻ is generated in situ by adding a salt containing Xⁿ⁻ to the reaction composition, wherein the salt containing Xⁿ⁻ has the formula:
M^{m+}(H⁺)₍ₙ₋ₘ₎Xⁿ⁻ or M^{m+}_{((n-q)/m)}(H⁺)_{q}Xⁿ⁻, and
wherein M^{m+} is a monovalent or polyvalent metal cation;
m and n are independently an integer selected from 1, 2, and 3, provided that m≦n; and
q is an integer selected from 0, 1, and 2, provided that q<n.

17. The process of any one of claims 8 to 14, wherein the compound of formula (Ia), the compound of formula (IIa), or the compounds of formulae (Ia) and (IIa) is obtained by adding a Lewis acid to the reaction composition instead of the acid H⁺ₙXⁿ⁻.

18. The process of any one of claims 14 to 17, wherein the amount of acid present is from about 0.5 to about 10 molar equivalents, from about 1 molar equivalent to about 6 molar equivalents, from about 2 to about 3 molar equivalents, or from about 2.2 to about 2.6 molar equivalents based on the total molar equivalent of compounds of formulae (I) and (II) or (Ia) and (IIa).

19. The process of any one of claims 14 to 18, wherein the acid H⁺ₙXⁿ⁻ of claims 14 to 16 or the Lewis acid of claim 17 is added to the reaction composition before hydrogenating step (b).

20. The process of any one of claims 8 to 19, wherein the compound of formula (Ia) is: 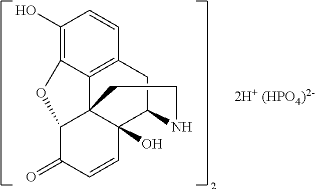
or a solvate thereof.

21. The process of any one of claims 8 to 19, wherein the compound of formula (Ia) is: 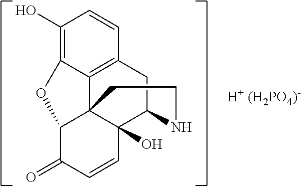
or a solvate thereof.

22. The process of any one of claims 8 to 19, wherein the compound of formula (Ia) is: 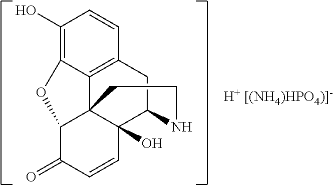
or a solvate thereof.

23. The process of any one of claims 9 to 22, wherein the compound of formula (IIa) is: 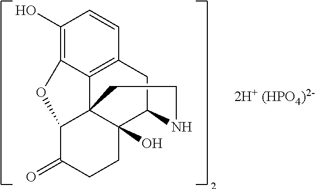
or a solvate thereof.

24. The process of any one of claims 9 to 22, wherein the compound of formula (IIa) is: 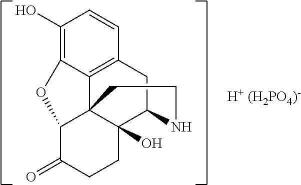
or a solvate thereof.

25. The process of any one of claims 9 to 22, wherein the compound of formula (IIa) is: 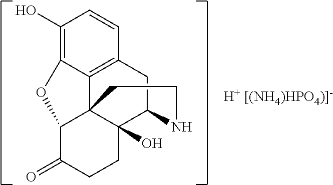
or a solvate thereof.

26. The process of any one of claims 1 to 26, wherein at least one of the compounds of formulae (I), (Ia), (II) and (IIa) or a solvate thereof is anhydrous or a hydrate of the compound of formula (I), (Ia), (II) or (IIa), respectively, and preferably is anhydrous.

27. The process of claim 26, wherein the hydrate contains from about 0.5 to about 5.0 water molecules per molecule of at least one of the compounds of formulae (I), (Ia), (II), and (IIa).

28. The process of claim 26 or 27, wherein the hydrate is a monohydrate, dihydrate, or trihydrate, of at least one of the compounds of formulae (I), (Ia), (II), and (IIa), and preferably is a dihydrate.

29. The process of any one of claims 1 to 28, wherein the reaction composition comprises a solvent.

30. The process of claim 29, wherein the solvent is selected from the group consisting of water, alcohols, aromatic hydrocarbons, aliphatic hydrocarbons, ethers, amides, N—(C₁-C₄)alkyl substituted (C₁-C₄)alkanoic acid amides, formylmorpholine, and mixtures thereof, wherein the aromatic hydrocarbons and the aliphatic hydrocarbons are optionally halogenated, the ether is preferably a (C₁-C₄)alkyl ester of a (C₁-C₄)alkanoic acid, and the amide is preferably N-methylpyrrolidone, dimethylformamide, or dimethylacetamide.

31. The process of claim 30, wherein the solvent is selected from the group consisting of water, ethers, alcohols, (C₁-C₄)alkanes, and mixtures thereof, wherein the (C₁-C₄)alkanes are optionally chlorinated.

32. The process of claim 30 or 31, wherein the solvent is selected from the group consisting of water, tetrahydrofuran, iso-propanol, methanol, ethanol, butanol, iso-butanol, tert-amylalcohol, n-propanol, chloroform, and mixtures thereof.

33. The process of any one of claims 29 to 32, wherein the amount of solvent present is from about 1 volume to about 20 volumes, from about 2 to about 10 volumes, from about 4.5 to about 10 volumes, or about 5 volumes based on the total mass of compounds of formulae (I) and (II).

34. The process of any one of claims 1 to 33, wherein hydrogenating step (b) is performed in the presence of a hydrogenation reagent.

35. The process of claim 34, wherein the hydrogenation reagent is hydrogen.

36. The process of claim 35, wherein the pressure of the hydrogen is from about 15×10⁴ Pa to about 100×10⁴ Pa, from about 30×10⁴ Pa to about 70×10⁴ Pa, or from about 45×10⁴ Pa to about 70×10⁴ Pa.

37. The process of any one of claims 34 to 36, wherein hydrogenating step (b) is performed in the presence of a hydrogenation catalyst.

38. The process of claim 37, wherein the hydrogenation catalyst is a transition-metal based hydrogenation catalyst, and preferably is selected from the group consisting of rhodium-based hydrogenation catalysts, ruthenium-based hydrogenation catalysts, platinum-based hydrogenation catalysts, palladium-based hydrogenation catalysts, and mixtures thereof.

39. The process of claim 38, wherein the transition-metal based hydrogenation catalyst is selected from the group consisting of palladium on carbon, palladium on BaSO₄, and palladium poisoned with sulfur on carbon.

40. The process of any one of claims 37 to 39, wherein the hydrogenation catalyst is selected from the group consisting of 5% palladium on carbon, 10% palladium on carbon, and mixtures thereof.

41. The process of any one of claims 37 to 40, wherein the amount of hydrogenation catalyst present is from about 0.1 to about 12.0 wt %, from about 1.5 to about 9.0 wt %, from about 1.7 to about 5.0 wt %, from about 1.8 to about 4.5 wt %, or from about 1.8 to about 2.5 wt % based on the total weight of compounds of formulae (I) and (II).

42. The process of claim 41, wherein the hydrogenation catalyst is 5% palladium on carbon and the amount of 5% palladium on carbon present is:
(i) at least about 1.5 wt %, at least about 5 wt %, at least about 10 wt %, or at least about 15 wt % based on the total weight of compounds of formulae (I) and (II); or
(ii) at least about 0.1 mol% based on the total moles of compounds of formulae (I) and (II).

43. The process of claim 41, wherein the hydrogenation catalyst is 10% palladium on carbon and the amount of 10% palladium on carbon present is:
(i) at least about 1.5 wt %, at least about 3.0 wt %, at least about 4.0 wt %, or at least about 5.0 wt % based on the total weight of compounds of formulae (I) and (II) or
(ii) at least about 0.2 mol% based on the total moles of compounds of formulae (I) and (II).

44. The process of any one of claims 34 to 43, wherein the amount of solvent present is from about 1 volume to about 20 volumes, from about 2 to about 10 volumes, from about 4 to about 10 volumes, or about 5 volumes based on the total mass of compounds of formulae (I) and (II).

45. The process of any one of claims 34 to 44, wherein hydrogenating step (b) is performed in the presence of an acid selected from the group consisting of H₂SO₄, H₃PO₄, HC(O)OH, and CH₃C(O)OH, and preferably is H₃PO₄.

46. The process of claim 45, wherein the amount of acid present is from about 0.5 to about 10 molar equivalents, from about 1 to about 6 molar equivalents, from about 2 to about 3 molar equivalents, or from about 2.2 to about 2.6 molar equivalents based on the total molar equivalent of compounds of formulae (I) and (II).

47. The process of any one of claims 34 to 46, wherein the temperature during hydrogenation in step (b) is from about 25° C. to about 110° C., from about 45° C. to about 100° C., or from about 75° C. to about 90° C.

48. The process of any one of claims 34 to 47, wherein the duration of hydrogenation in step (b) is from about 1 hour to about 96 hours, from about 2 to about 48 hours, or from about 4 to about 10 hours.

49. The process of any one of claims 34 to 48, wherein the temperature during hydrogenation in step (b) is from about 75° C. to about 85° C., and the duration of hydrogenation in step (b) is from about 4 to about 10 hours.

50. The process of any one of claims 34 to 49, wherein hydrogenating step (b) is performed in the presence of a halide-containing compound.

51. The process of claim 50, wherein the halide-containing compound is selected from the group consisting of ammonium chloride, sodium iodide, sodium chloride, sodium bromide and hydrochloric acid, and preferably is sodium iodide, sodium chloride or sodium bromide, and more preferably is sodium iodide.

52. The process of claim 50 or 51, wherein the amount of halide-containing compound present is from about 0.0001 to about 15.0 wt %, from about 1.0 to about 12.0 wt %, from about 2.5 to about 10.0 wt %, from about 3.5 to about 7.5 wt %, or from about 4.5 to about 5.0 wt % based on the total weight of compounds of formulae (I) and (II).

53. The process of claim 50 or 51, wherein the halide-containing compound is sodium iodide present in an amount of at least 250 ppm, at least 500 ppm or at least 1000 ppm based on the total weight of compounds of formulae (I) and (II).

54. The process of claim 50 or 51, wherein the halide-containing compound is sodium iodide present in an amount of from about 0.0001 to about 15 wt %, from about 0.001 to about 1 wt %, or from about 0.0025 to about 0.1 wt % based on the total weight of compounds of formulae (I) and (II).

55. The process of claim 50 or 51, wherein the halide-containing compound is sodium chloride present in an amount from about 0.5 to about 15.0 wt %, from about 1.0 to about 12.0 wt %, from about 2.5 to about 10.0 wt %, from about 3.5 to about 7.5 wt %, or from about 4.5 to about 5.0 wt % based on the total weight of compounds of formulae (I) and (II).

56. The process of any one of claims 50 to 55, wherein the addition of the halide-containing compound is performed before the addition of the hydrogenation catalyst.

57. The process of any one of claims 1 to 56, further comprising addition of a base in salt-breaking step (c) after the hydrogenation reaction of step (b).

58. The process of claim 57, wherein the base is selected from the group consisting of sodium hydroxide, potassium hydroxide, aluminum hydroxide, ammonia and ammonium hydroxide, and preferably is ammonium hydroxide.

59. The process of claim 57 or 58, wherein the pH after addition of the base is from about 7.0 to about 12.0, from about 7.5 to about 9.5, or from about 8.0 to about 9.0.

60. The process of any one of claims 57 to 59, wherein at least a portion of the base in salt-breaking step (c) is added to the product of hydrogenating step (b) wherein during base addition said product is at a temperature from about 0° C. to about 100° C., from about 30° C. to about 100° C., or from about 40° C. to about 90° C.

61. The process of any one of claims 57 to 60, wherein at least a first portion of the base in salt-breaking step (c) is added until a pH of from about 2.0 to about 6.0 or from about 4.5 to about 5.5 is reached.

62. The process of claim 61, wherein the temperature of the product of hydrogenating step (b) during said addition of at least the first portion of the base is from about 15° C. to about 50° C., from about 20° C. to about 30° C., or about 25° C.

63. The process of any one of claims 57 to 62, wherein the base is added in two portions:
i) a first portion of the base is added to the product of hydrogenating step (b) while the temperature is from about 15° C. to about 50° C. or from about 20° C. to about 30° C., until the pH is from about 2.0 to about 6.0, from about 4.5 to about 5.5, or about 5.0; and
ii) a second portion of the base is added wherein during base addition said the temperature is of from about 40° C. to about 90° C., from about 70° C. to about 80° C., or about 75° C., until a pH of from about 7.0 to about 9.5 is reached.

64. The process of any one of claims 57 to 63, further comprising crystallization or precipitation of a composition comprising compounds of formulae (I) and (II), or salts or solvates thereof, from the reaction mixture.

65. The process of claim 64, wherein crystallization or precipitation is induced by at least one of the following:
i) adjusting the temperature of the composition;
ii) adding an antisolvent to the composition;
iii) adding a seed crystal to the composition;
iv) adjusting the pH of the composition;
v) adding a salt to the composition;
vi) concentrating the composition; or
vii) reducing or stopping agitation of the composition.

66. The process of any one of claims 57 to 65, wherein an isolating step (d) providing a residue is performed after salt-breaking step (c), wherein the isolating step (d) is preferably a filtration step.

67. The process of claim 66, wherein the temperature of the composition before the isolating step (d) is from about 5° C. to about 90° C., from about 20° C. to about 70° C., or from about 40° C. to about 50° C.

68. The process of claim 66 or 67, wherein the isolating step (d) is a filtration step further comprising washing the residue comprising compounds of formulae (I) and (II) with a washing solvent selected from the group consisting of water, methanol, ethanol, iso-propanol, acids, and mixtures thereof and preferably the washing solvent is selected from the group consisting of water, methanol, ethanol, iso-propanol, H₂SO₄, H₃PO₄, HC(O)OH, CH₃C(O)OH, and mixtures thereof.

69. The process of claim 68, wherein the amount of washing solvent present is from about 0.1 to about 12 volumes, from about 0.5 to about 8 volumes, from about 1 volume to about 4 volumes, or about 2 volumes based on the total mass of the filtration residue.

70. The process of any one of claims 57 to 69, wherein a drying step is performed after salt-breaking step (c) and wherein the drying step is preferably performed at a temperature of from about 40° C. to about 100° C.

71. The process of claim 70, wherein the water content of the composition comprising compounds of formulae (I) and (II), or the salts or solvates thereof, in the product after drying is less than about 20 wt %, less than about 13 wt %, less than about 11 wt %, less than about 5 wt %, or less than about 1 wt % based, on the total weight of compounds of formulae (I) and (II) or the salt or the solvate thereof.

72. The process of any one of claims 1 to 71, further comprising step (a), which is the addition of a decolorizing agent to the composition comprising compounds of formulae (I) and (II) or the salt or the solvate thereof.

73. The process of claim 72, wherein decolorizing step (a) is performed in the sequence of at least one of before, during, and after hydrogenating step (b).

74. The process of claim 72 or 73, wherein decolorizing step (a) and hydrogenating step (b) are performed simultaneously.

75. The process of any one of claims 72 to 74, wherein the decolorizing agent is selected from the group consisting of a carbon-based decolorizing agent, an aluminum-based decolorizing agent, and mixtures thereof, and preferably is a carbon-based decolorizing agent.

76. The process of claim 75, wherein the aluminum-based decolorizing agent is Al₂O₃.

77. The process of any one of claims 72 to 76, wherein the amount of decolorizing agent present is from about 10 to about 80 wt %, from about 15 to about 60 wt %, from about 20 wt % to about 30 wt %, or about 25 wt % based on the total weight of compounds of formulae (I) and (II) or the salt or the solvate thereof.

78. The process of any one of claims 72 to 77, wherein decolorizing step (a) is performed in the presence of a solvent selected from the group consisting of water, alcohols, aromatic hydrocarbons, aliphatic hydrocarbons, ethers, amides, N—(C₁-C₄)alkyl substituted (C₁-C₄)alkanoic acid amides, formylmorpholine, and mixtures thereof, wherein the aromatic hydrocarbons and the aliphatic hydrocarbons are optionally halogenated, the ether is preferably a (C₁-C₄)alkyl ester of a (C₁-C₄)alkanoic acid, and the amide is preferably N-methylpyrrolidone, dimethylformamide, or dimethylacetamide.

79. The process of claim 78, wherein the amount of solvent present is from about 1 volume to about 12 volumes, from about 2 to about 8 volumes, from about 3 to about 6 volumes, or about 5 volumes based on the total mass of compounds of formulae (I) and (II) or the salt or the solvate thereof and wherein the solvent is preferably selected from the group consisting of water, tetrahydrofuran, iso-propanol, methanol, ethanol, butanol, iso-butanol, tert-amylalcohol, n-propanol, chloroform, and mixtures thereof.

80. The process of any one of claims 72 to 79, wherein decolorizing step (a) is performed in the presence of an acid selected from the group consisting of H₂SO₄, H₃PO₄, HC(O)OH, and CH₃C(O)OH, and preferably is H₃PO₄.

81. The process of claim 80, wherein the amount of acid present is from about 0.5 to about 10 molar equivalents, from about 1 molar equivalent to about 6 molar equivalents, from about 2 to about 3 molar equivalents, or from about 2.2 to about 2.6 molar equivalents based on the total molar equivalent of compounds of formulae (I) and (II) or the salt or the solvate thereof.

82. The process of any one of claims 72 to 81, wherein decolorizing step (a) is performed at a temperature of from about 30° C. to about 105° C., preferably from about 70° C. to about 105° C., and more preferably from about 75° C. to about 90° C.

83. The process of any one of claims 72 to 82, wherein the transparency of the composition comprising compounds of formulae (I) and (II) or the salt or the solvate thereof is increased.

84. The process of claim 83, wherein the yellowness index determined according to Equation 4 for the composition comprising compounds of formulae (I) and (II) or the salt or the solvate thereof in the product is less than about 100, less than about 50, less than about 25, or less than about 10 at a concentration of the composition comprising compounds of formulae (I) and (II) or the salt or the solvate thereof of about 4 mg/mL in an aqueous H₃PO₄ solution.

85. The process of any one of claims 72 to 84, wherein a filtration step is performed after decolorizing step (a) to remove the decolorizing agent preferably as a filter cake.

86. The process of claim 85, wherein the temperature of the liquid being filtered is from about 15° C. to about 110° C., from about 30° C. to about 90° C., from about 50° C. to about 70° C., or about 60° C.

87. The process of claim 85 or 86, wherein the filtration step comprises washing the filter cake obtained from the filtration with a wash solvent selected from the group consisting of water, alcohol, and mixtures thereof, and preferably washing with water.

88. The process of claim 87, wherein the volume of the wash solvent is from about 1 volume to about 10 volumes, from about 1 volume to about 5 volumes, or about 2 volumes based on the total mass of the filter cake.

89. The process of any one of claims 1, 2, 4, and 8 to 13, wherein the compound of formula (I) is 14-hydroxynormorphinone or a salt or a solvate thereof, the compound of formula (II) is noroxymorphone or a salt or a solvate thereof, and the hydrogenation catalyst in hydrogenating step (b) is 5 wt % palladium on carbon.

90. The process of any one of claims 14 to 88, wherein the compound of formula (I) is 14-hydroxynormorphinone or a salt or a solvate thereof, H₃PO₄ is added to the reaction composition, the compound of formula (II) is noroxymorphone or a salt or a solvate thereof, and the hydrogenation catalyst in hydrogenating step (b) is 5 wt % palladium on carbon.

91. The process of claim 89 or 90, wherein the amount of 5 wt % palladium on carbon present is about 1.8 wt % based on the total weight of compounds of formulae (I) and (II) or a salt or a solvate thereof.

92. The process of claim 90 or 91, wherein the amount of H₃PO₄ present is from about 2.2 to about 2.6 molar equivalents based on the total molar equivalent of compounds of formulae (I) and (II) or a salt or a solvate thereof.

93. The process of any one of claims 89 to 92, wherein the hydrogenation process is performed in the presence of a halide-containing compound.

94. The process of claim 93, wherein the halide-containing compound is sodium iodide and, preferably, the amount of sodium iodide present is about 0.1 wt. % based on the total weight of compounds of formulae (I) and (II) or a salt or a solvate thereof.

95. The process of claim 93, wherein the halide-containing compound is sodium chloride and, preferably, the amount of sodium chloride present is about 5 wt. % based on the total weight of compounds of formulae (I) and (II) or a salt or a solvate thereof.

96. The process of any one of claims 89 to 95 further comprising addition of a base in salt-breaking step (c) after the hydrogenation reaction of step (b), wherein the base in salt-breaking step (c) is ammonium hydroxide.

97. The process of any one of claims 89 to 96, wherein the base is added in two portions:
i) a first portion of base is added to the product of hydrogenating step (b) while the temperature is from about 20° C. to about 30° C., until the pH is from about 4.5 to about 5.5; and
ii) a second portion of base is added wherein during base addition the temperature is of from about 70° C. to about 80° C., until a pH of from about 7.5 to about 8.5 is reached.

98. The process of any one of claims 89 to 97 further comprising a decolorizing step (a) comprising addition of a decolorizing agent, wherein decolorizing step (a) is performed before hydrogenating step (b).

99. The process of any one of claims 89 to 98, wherein activated carbon is used as the decolorizing agent in decolorizing step (a).

100. The process of claim 99, wherein the amount of activated carbon present is about 25 wt % based on the total weight of compounds of formulae (I) and (II).

101. The process of any of claims 1 to 100, wherein the process further comprises reducing the amount of a compound of formula (III) present: 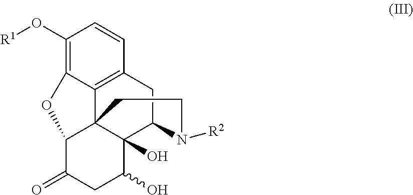
or a salt or a solvate thereof;
wherein le and R² are defined as in any one of claims 1 to 6.

102. The process of claim 101, wherein R² is —H.

103. The process of claim 101, wherein R² is —CH₂CH═CH₂ or —CH₂-cyclopropyl.

104. The process of claim 101 or 102, wherein the compound of formula (III) is: 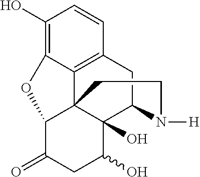 [SMILES: O=C1CC(O)[C@@]2(O)[C@H]3Cc4ccc(O)c5c4[C@@]2(CCN3)[C@H]1O5]
or a salt or a solvate thereof.

105. The process of any one of claims 101 to 104, wherein the compound of formula (III) is a compound of formula (Ma): 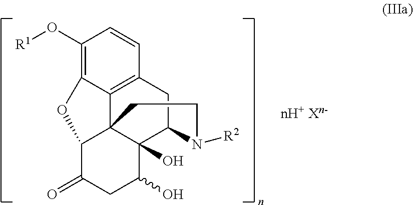
or a solvate thereof;
wherein R¹, R², Xⁿ⁻ and n are defined as in any one of claims 1 to 6 and 8 to 13.

106. The process of any of claims 1 to 105, wherein the process further comprises reducing the amount of a compound of formula (IV) present: 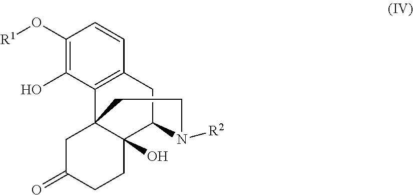
or a salt or a solvate thereof;
wherein R¹ and R² are defined as in any one of claims 1 to 6.

107. The process of claim 106, wherein the compound of formula (IV) is a compound of formula (IVa): 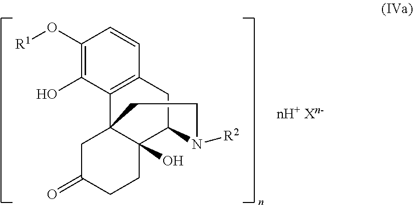
or a solvate thereof;
wherein R¹, R², Xⁿ⁻ and n are defined as in any one of claims 1 to 6 and 8 to 13.

108. The process of any one of claims 1 to 107, wherein the amount present of the compound of formula (I) or a salt or a solvate thereof relative to the amount of the compound of formula (II) or a salt or a solvate thereof in the product is less than about 200 ppm, less than about 150 ppm, less than about 100 ppm, less than about 75 ppm, less than about 50 ppm, less than about 40 ppm, less than about 35 ppm, less than about 25 ppm, less than about 10 ppm, or less than 5 ppm.

109. The process of any one of claims 1 to 108, wherein the compound having the formula (I) is 14-hydroxynormorphinone or a salt or a solvate thereof, wherein the compound having the formula (II) is noroxymorphone or a salt or a solvate thereof, and wherein the amount present of 14-hydroxynormorphinone or a salt or a solvate thereof relative to the amount of noroxymorphone or a salt or a solvate thereof in the product is less than about 200 ppm, less than about 150 ppm, less than about 100 ppm, less than about 75 ppm, less than about 50 ppm, less than about 40 ppm, less than about 35 ppm, less than about 25 ppm, less than about 10 ppm, or less than 5 ppm, each preferably as determined by the HPLC method of Example 1.1.

110. The process of any one of claims 1 to 109, wherein the compound having the formula (I) is 7,8-didehydronaloxone or a salt or a solvate thereof, wherein the compound having the formula (II) is naloxone or a salt or a solvate thereof, and wherein the amount present of 7,8-didehydronaloxone or a salt or a solvate thereof relative to the amount of naloxone or a salt or a solvate thereof in the product is less than about 200 ppm, less than about 150 ppm, less than about 100 ppm, less than about 75 ppm, less than about 50 ppm, less than about 40 ppm, less than about 35 ppm, less than about 25 ppm, less than about 10 ppm, or less than 5 ppm, each preferably as determined by the HPLC method of Example 1.3.

111. The process of any one of claims 1 to 109, wherein the compound having the formula (I) is 7,8-didehydronaltrexone or a salt or a solvate thereof, wherein the compound having the formula (II) is naltrexone or a salt or a solvate thereof, and wherein the amount present of 7,8-didehydronaltrexone or a salt or a solvate thereof relative to the amount of naltrexone or a salt or a solvate thereof in the product is less than about 200 ppm, less than about 150 ppm, less than about 100 ppm, less than about 75 ppm, less than about 50 ppm, less than about 40 ppm, less than about 35 ppm, less than about 25 ppm, less than about 10 ppm, or less than 5 ppm, each preferably as determined by the HPLC method of Example 1.4.

112. The process of any one of claims 1 to 111 wherein the yellowness index determined according to Equation 4 for the composition comprising compounds of formulae (I) and (II) or the salt or the solvate thereof in the product is less than about 100, preferably less than about 50, and more preferably less than about 25 at a concentration of the composition comprising compounds of formulae (I) and (II) or the salt or the solvate thereof of about 4 mg/mL in an aqueous H₃PO₄ solution.

113. The process of any one of claims 1 to 112, wherein the amount present of the compound of formula (IV) or the salt or the solvate thereof relative to the amount of the compound of formula (II) or the salt or the solvate thereof in the product is less than about 0.5 HPLC peak area ratio, preferably less than about 0.25 HPLC peak area ratio and most preferably less than about 0.15 HPLC peak area ratio, wherein preferably each HPLC peak area is determined according to the procedure provided in Example 1.2.

114. The process of any one of claims 1 to 113, wherein the amount present of the compound of formula (I) or a salt or a solvate thereof in the product is less than about 200 ppm, less than about 100 ppm, less than about 75 ppm, less than about 50 ppm, less than about 25, less than about 10 ppm, or less than 5 ppm, relative to the amount of the compound of formula (II) or a salt or a solvate thereof, and an amount present of the compound of formula (IV) or a salt or a solvate thereof is less than about 0.5 HPLC peak area ratio, less than about 0.25 HPLC peak area ratio, or less than about 0.15 HPLC peak area ratio relative to the amount of the compound of formula (II) or the salt or the solvate thereof in the product, wherein preferably each HPLC peak area is determined according to the procedure provided in Example 1.2.

115. The process of any one of claims 1 to 114, wherein the amount of present the compound of formula (I) or a salt or a solvate thereof relative to the amount of the compound of formula (II) or a salt or a solvate thereof in the product is from about 5 ppm to less than about 200 ppm, from about 5 ppm to less than about 150 ppm, from about 5 ppm to less than about 100 ppm, from about 5 ppm to less than about 75 ppm, from about 5 ppm to less than about 50 ppm, from about 5 ppm to less than about 40 ppm, from about 5 ppm to less than about 35 ppm, or from about 5 ppm to less than about 25 ppm.

116. The process of any one of claims 1 to 115, wherein the amount of present the compound of formula (I) or a salt or a solvate thereof relative to the amount of the compound of formula (II) or a salt or a solvate thereof in the product is from about 10 ppm to less than about 200 ppm, from about 10 ppm to less than about 150 ppm, from about 10 ppm to less than about 100 ppm, from about 10 ppm to less than about 75 ppm, from about 10 ppm to less than about 50 ppm, from about 5 ppm to less than about 40 ppm,from about 10 ppm to less than about 35 ppm, or from about 10 ppm to less than about 25 ppm.

117. A composition comprising compounds of formulae (I) and (II): 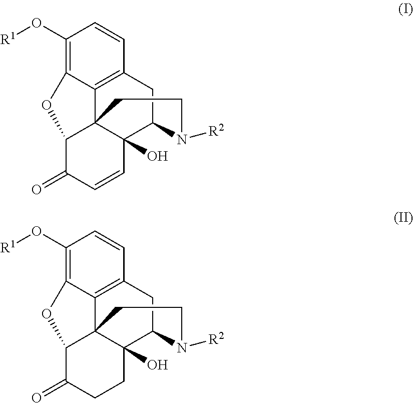
or a pharmaceutically acceptable salt or solvate thereof;
wherein:
R¹ and R² are defined as in any one of claims 1 to 6; and
the amount of the compound of formula (I) present in the composition relative to the amount of the compound of formula (II) is less than about 200 ppm, less than about 150 ppm, less than about 100 ppm, less than about 75 ppm, less than about 50 ppm, from about 5 ppm to less than about 40 ppm, less than about 35 ppm, less than about 25 ppm, less than about 10 ppm, or less than 5 ppm.

118. The composition of claim 117, wherein R¹ is —H.

119. The composition of claim 117 or 118, wherein R² is —H.

120. The composition of claim 119, wherein the amount of formula (I) present in the composition relative to the amount of the compound of formula (II) is less than about 200 ppm, less than about 150 ppm, less than about 100 ppm, less than about 75 ppm, less than about 50 ppm, from about 5 ppm to less than about 40 ppm,less than about 35 ppm, less than about 25 ppm, less than about 10 ppm, or less than 5 ppm, preferably each as determined by the HPLC method of Example 1.1.

121. The composition of claim 117 or 118, wherein R² is —CH₂CH═CH₂ or —CH₂-cyclopropyl.

122. The composition of claim 118 or 121, wherein R² is —CH₂CH═CH₂ and wherein the amount of the compound of formula (I) present relative to the amount of the compound of formula (II) is less than about 200 ppm, less than about 150 ppm, less than about 100 ppm, less than about 75 ppm, less than about 50 ppm, less than about 40 ppm, less than about 35 ppm, less than about 25 ppm, less than about 10 ppm, or less than 5 ppm, preferably each as determined by the HPLC method of Example 1.3.

123. The composition of claim 118 or 121, wherein R² is —CH₂-cyclopropyl and wherein the amount of the compound of formula (I) present relative to the amount of the compound of formula (II) is less than about 200 ppm, less than about 150 ppm, less than about 100 ppm, less than about 75 ppm, less than about 50 ppm, less than about 40 ppm, less than about 35 ppm, less than about 25 ppm, less than about 10 ppm, or less than 5 ppm, preferably each as determined by the HPLC method of Example 1.4.

124. The composition of any one of claims 117 to 120, wherein the compound of formula (I) is: 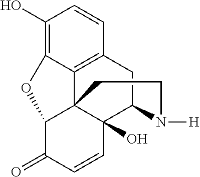 [SMILES: O=C1C=C[C@@]2(O)[C@H]3Cc4ccc(O)c5c4[C@@]2(CCN3)[C@H]1O5]
or a pharmaceutically acceptable salt thereof,
and the compound of formula (II) is: 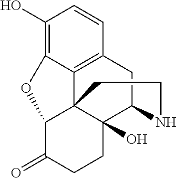 [SMILES: O=C1CC[C@@]2(O)[C@H]3Cc4ccc(O)c5c4[C@@]2(CCN3)[C@H]1O5]
or a pharmaceutically acceptable salt thereof.

125. The composition of any one of claims 117, 121 and 122, wherein the compound of formula (I) is: 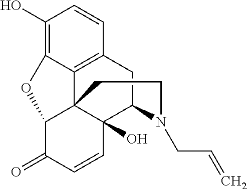 [SMILES: C=CCN1CC[C@]23c4c5ccc(O)c4O[C@H]2C(=O)C=C[C@@]3(O)[C@H]1C5]
or a pharmaceutically acceptable salt or solvate thereof,
and the compound of formula (II) is: 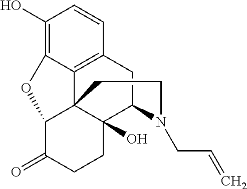 [SMILES: C=CCN1CC[C@]23c4c5ccc(O)c4O[C@H]2C(=O)CC[C@@]3(O)[C@H]1C5]
or a pharmaceutically acceptable salt or solvate thereof.

126. The composition of any one of claims 117, 121 and 122, wherein the compound of formula (I) is: 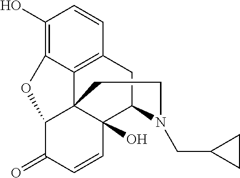 [SMILES: O=C1C=C[C@@]2(O)[C@H]3Cc4ccc(O)c5c4[C@@]2(CCN3CC2CC2)[C@H]1O5]
or a pharmaceutically acceptable salt or solvate thereof,
and the compound of formula (II) is: 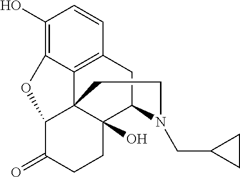 [SMILES: O=C1CC[C@@]2(O)[C@H]3Cc4ccc(O)c5c4[C@@]2(CCN3CC2CC2)[C@H]1O5]
or a pharmaceutically acceptable salt or solvate thereof.

127. The composition of any one of claims 117 to 124, wherein the compound of formula (I) is a compound of formula (Ia): 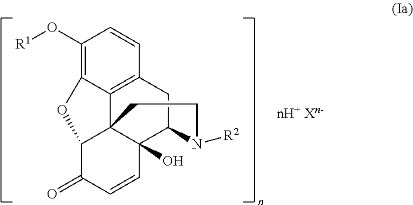
or a solvate thereof;
wherein R¹, R², Xⁿ⁻ and n are defined as in any of claims 1 to 6 and 8 to 13.

128. The composition of any one of claims 117, and 122 to 125, wherein the compound of formula (II) is a compound of formula (IIa): 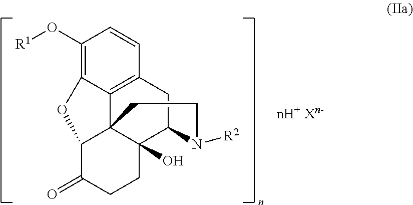
or a solvate thereof;
wherein R¹, R², Xⁿ⁻ and n are defined as in any one of claims 1 to 6 and 8 to 13.

129. The composition of claim 127 or 128, wherein Xⁿ⁻ is selected from the group consisting of HSO₄⁻, SO₄²⁻, H₂PO₄²⁻, HPO₄²⁻, [(NH₄)HPO₄]⁻, H₃CC(O)O⁻, HC(O)O⁻, and mixtures thereof.

130. The composition of claim 129, wherein Xⁿ⁻ is selected from the group consisting of H₂PO₄⁻, HPO₄²⁻, [(NH₄)HPO₄]⁻, H₃CC(O)O⁻, HC(O)O⁻, and mixtures thereof.

131. The composition of claim 129 or 130, wherein Xⁿ⁻ is selected from the group consisting of H₂PO₄⁻, HPO₄²⁻, [(NH₄)HPO₄]⁻, and mixtures thereof.

132. The composition of any one of claims 126 to 131, wherein the compound of formula (Ia), the compound of formula (IIa), or the compound of formula (Ia) and the compound of formula (IIa) is obtained by adding an acid H⁺ₙXⁿ⁻ to the reaction composition, wherein said acid H⁺ₙXⁿ⁻ is preferably H₃PO₄.

133. The composition of any one of claims 126 to 132, wherein n is 1.

134. The composition of any one of claims 126 to 132, wherein n is 2.

135. The composition of any one of claims 127 to 133, wherein the compound of formula (Ia) is: 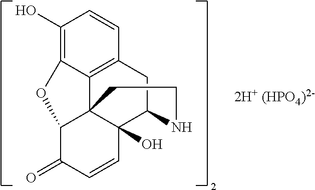
or a solvate thereof.

136. The composition of any one of claims 127 to 134, wherein the compound of formula (Ia) is: 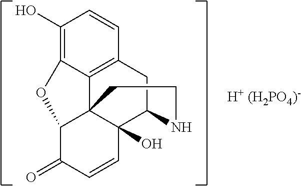
or a solvate thereof.

137. The composition of any one of claims 127 to 134, wherein the compound of formula (Ia) is: 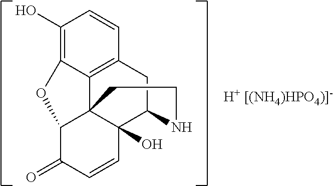
or a solvate thereof.

138. The composition of any one of claims 127 to 133, wherein the compound of formula (Ia) is: 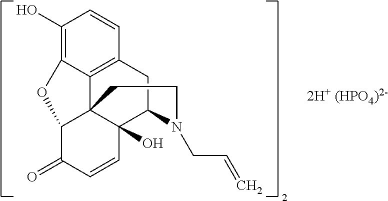
or a solvate thereof.

139. The composition of any one of claims 127 to 134, wherein the compound of formula (Ia) is: 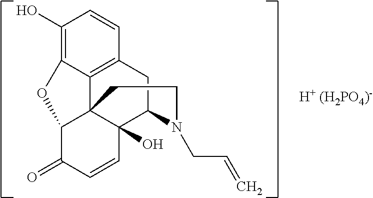
or a solvate thereof.

140. The composition of any one of claims 127 to 134, wherein the compound of formula (Ia) is: 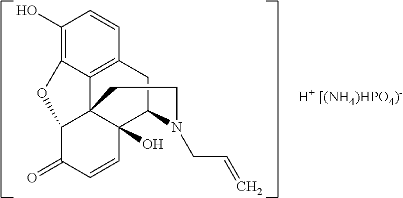
or a solvate thereof.

141. The composition of any one of claims 127 to 139, wherein the compound of formula (IIa) is: 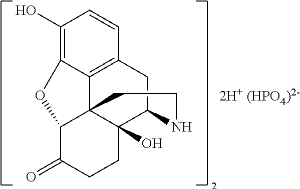
or a solvate thereof.

142. The composition of any one of claims 127 to 134, wherein the compound of formula (IIa) is: 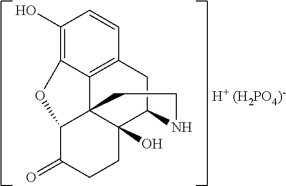
or a solvate thereof.

143. The composition of any one of claims 127 to 134, wherein the compound of formula (IIa) is: 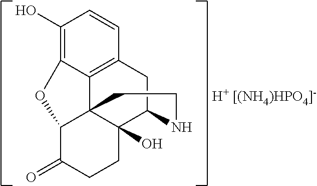
or a solvate thereof.

144. The composition of any one of claims 127 to 133, wherein the compound of formula (IIa) is: 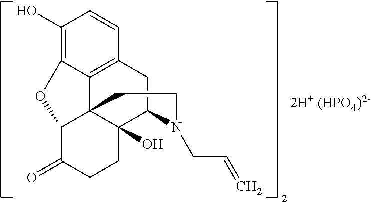
or a solvate thereof.

145. The composition of any one of claims 127 to 134, wherein the compound of formula (IIa) is: 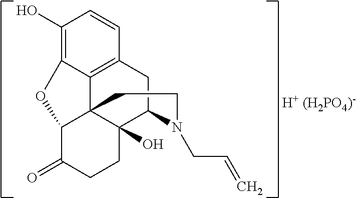
or a solvate thereof.

146. The composition of any one of claims 127 to 134, wherein the compound of formula (IIa) is: 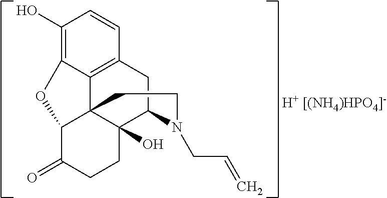
or a solvate thereof.

147. The composition of any one of claims 127 to 134, wherein the compound of formula (IIa) is: 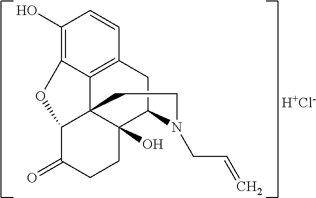
or a solvate thereof.

148. The composition of any one of claims 117 to 147, wherein at least one of the compounds of formulae (I), (Ia), (II), or (IIa) is anhydrous or is a hydrate of the compound of formulae (I), (Ia), (II), or (IIa)

149. The composition of claim 148, wherein the hydrate contains from about 0.5 to about 5.0 water molecules per molecule of the compound of formula (I), the compound of formula (Ia), the compound of formula (II), the compound of formula (IIa), or at least one of the compounds of formulae (I), (Ia), (II), and (IIa).

150. The composition of claim 148 or 149, wherein the hydrate is a monohydrate, a dihydrate, or a trihydrate of the compound of formula (I), the compound of formula (Ia), the compound of formula (II), the compound of formula (IIa), or at least one of the compounds of formulae (I), (Ia), (II), and (IIa).

151. The composition of any one of claims 117 to 150, wherein the composition further comprises a compound of formula (III): 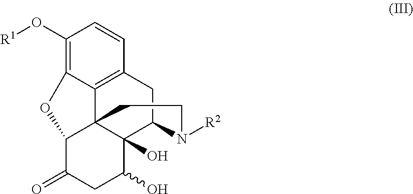
or a pharmaceutically acceptable salt or solvate thereof;
wherein R¹ and R² are defined as in any of claims 1 to 6.

152. The composition of claim 151, wherein the compound of formula (III) is a compound of formula (IIIa): 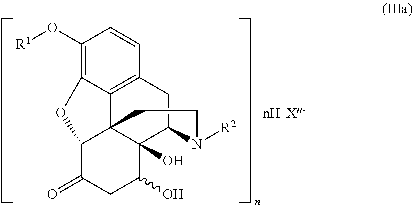
or a solvate thereof;
wherein R¹, R², Xⁿ⁻ and n are defined as in any one of claims 1 to 6 and 8 to 13.

153. The composition of any one of claims 117 to 152, wherein the composition further comprises a compound of formula (IV): 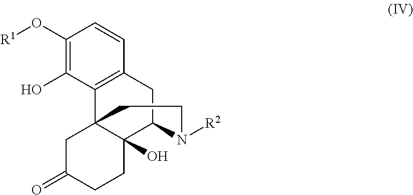
or a salt or a solvate thereof;
wherein R¹ and R² are defined as in any one of claims 1 to 6.

154. The composition of claim 153, wherein the compound of formula (IV) is a compound of formula (IVa): 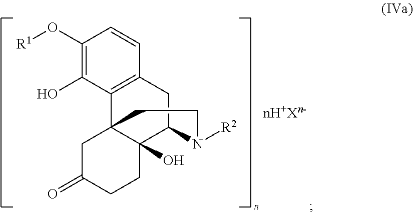
wherein R¹, R², Xⁿ⁻ and n are defined as in any one of claims 1 to 6 and 8 to 13.

155. The composition of claim 153 or 154, wherein the amount present of the compound of formula (I) or a salt or a solvate thereof in the product is less than about 200 ppm, less than about 100 ppm, less than about 75 ppm, less than about 50 ppm, less than about 25 ppm, less than about 10 ppm, or less than 5 ppm relative to the amount of the compound of formula (II) or a salt or a solvate thereof and an amount present of the compound of formula (IV) or a salt or a solvate thereof is less than about 0.5 HPLC peak area ratio, less than about 0.25 HPLC peak area ratio, or less than about 0.15 HPLC peak area ratio relative to the amount of the compound of formula (II) or the salt or the solvate thereof in the product, wherein preferably each HPLC peak area is determined according to the procedure provided in Example 1.2.

156. The composition of any one of claims 117 to 155, wherein the amount present of the compound of formula (I) or a salt or a solvate thereof in the product relative to the amount of the compound of formula (II) in the composition is from about 5 ppm to less than about 200 ppm, from about 5 ppm to less than about 150 ppm, from about 5 ppm to less than about 100 ppm, from about 5 ppm to less than about 75 ppm, from about 5 ppm to less than about 50 ppm, from about 5 ppm to less than about 40 ppm, from about 5 ppm to less than about 35 ppm, or from about 5 ppm to less than about 25 ppm.

157. The composition of any one of claims 117 to 156, wherein the amount present of the compound of formula (I) or a salt or a solvate thereof in the product relative to the amount of the compound of formula (II) in the composition is from about 10 ppm to less than about 200 ppm, from about 10 ppm to less than about 150 ppm, from about 10 ppm to less than about 100 ppm, from about 10 ppm to less than about 75 ppm, from about 10 ppm to less than about 50 ppm, from about 10 ppm to less than about 40 ppm, from about 10 ppm to less than about 35 ppm, or from about 10 ppm to less than about 25 ppm.

158. A composition comprising compounds of formulae (I) and (II): 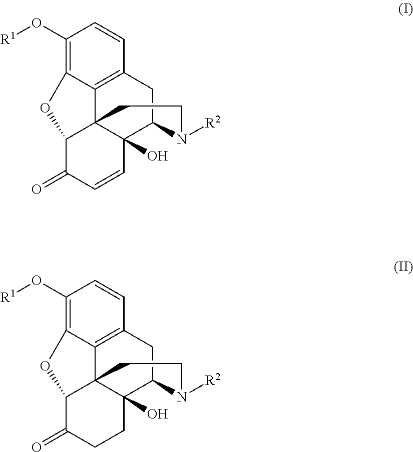
or a pharmaceutically acceptable salt or solvate thereof;
wherein R¹ and R² are defined as in any one of claims 1 to 6;
obtainable by the process of any one of claims 1 to 107;
wherein the amount of the compound of formula (I) present in the composition relative to the amount of the compound of formula (II) is less than about 200 ppm, less than about 150 ppm, less than about 100 ppm, less than about 50 ppm, less than about 40 ppm, less than about 35 ppm, less than about 25 ppm, less than about 10 ppm, or less than 5 ppm.

159. The composition of claim 158, wherein the composition additionally comprises a compound of formula (IV): 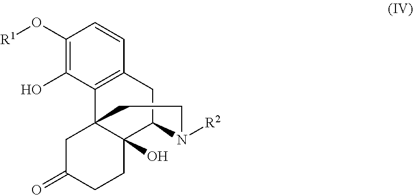
or a pharmaceutically acceptable salt or solvate thereof;
wherein R¹ and R² are defined as in any one of claims 1 to 6.

160. The composition of claim 159, wherein the composition additionally comprises a compound of formula (IV): 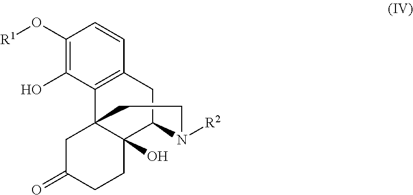
or a pharmaceutically acceptable salt or solvate thereof;
wherein R¹ and R² are defined as in any one of claims 1 to 6;
wherein the amount of the compound of formula (I) present in the composition relative to the amount of the compound of formula (II) is less than about 200 ppm, less than about 150 ppm, less than about 100 ppm, less than about 50 ppm, less than about 40 ppm, less than about 35 ppm, less than about 25 ppm, less than about 10 ppm, or less than 5 ppm, and wherein the amount present of the compound of formula (IV) or pharmaceutically acceptable salt or solvate thereof is less than about 0.5 HPLC peak area ratio, preferably less than about 0.25 HPLC peak area ratio, and most preferably less than about 0.15 HPLC peak area ratio relative to the amount of the compound of formula (II) or the salt or the solvate thereof, wherein preferably each HPLC peak area is determined according to the procedure provided in Example 1.2.

161. The composition of any one of claims 158 to 160, wherein the composition additionally comprises a compound of formula (III): 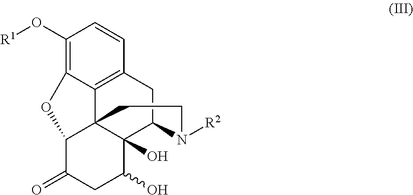
or a pharmaceutically acceptable salt or solvate thereof;
wherein R¹ and R² are defined as in any one of claims 1 to 6.

162. The composition of any one of claims 158 to 161, wherein the amount of the compound of formula (I) present in the composition relative to the amount of the compound of formula (II) is from about 5 ppm to less than about 200 ppm, from about 5 ppm to less than about 150 ppm, from about 5 ppm to less than about 100 ppm, from about 5 ppm to less than about 50 ppm, from about 5 ppm to less than about 40 ppm, from about 5 ppm to less than about 35 ppm, or from about 5 ppm to less than about 25 ppm.

163. The composition of any one of claims 158 to 161, wherein the amount of the compound of formula (I) present in the composition relative to the amount of the compound of formula (II) is from about 10 ppm to less than about 200 ppm, from about 10 ppm to less than about 150 ppm, from about 10 ppm to less than about 100 ppm, from about 10 ppm to less than about 50 ppm, from about 10 ppm to less than about 50 ppm, from about 10 ppm to less than about 35 ppm, or from about 10 ppm to less than about 25 ppm.

164. A process for preparing naloxone or a pharmaceutically acceptable salt or solvate thereof, comprising the steps of:
(i) providing a composition comprising compounds of formulae (I) and (II) or a pharmaceutically acceptable salt or solvate thereof, wherein R¹ and R² are defined as in any one of claims 1 to 6 and wherein the amount of the compound of formula (I) present in the composition relative to the amount of the compound of formula (II) is less than about 100 ppm, less than about 75 ppm, less than about 50 ppm, less than about 25 ppm, or less than about 10 ppm; and
(ii) reacting the composition of (i) with an alkylating agent to form naloxone or a pharmaceutically acceptable salt or solvate thereof, wherein the amount of 7,8-didehydronaloxone present relative to the amount of naloxone is less than about 100 ppm, less than about 75 ppm, less than about 50 ppm, less than about 25 ppm, or less than about 10 ppm.

165. The process of claim 164, wherein the alkylating agent is selected from the group consisting of allyl halides and is preferably allyl bromide.

166. The process of claim 164 or 165, wherein the amount of 7,8-didehydronaloxone present is determined by the HPLC method of Example 1.3.

167. The process of any one of claims 164 to 166, wherein the amount of the compound of formula (I) present in the composition relative to the amount of the compound of formula (II) is from about 10 ppm to less than about 100 ppm, from about 10 ppm to less than about 75 ppm, from about 10 ppm to less than about 50 ppm, from about 10 ppm to less than about 40 ppm,or from about 10 ppm to less than about 25 ppm, each as determined by the HPLC method of Example 1.3.

168. A process for preparing naltrexone or a pharmaceutically acceptable salt or solvate thereof, comprising the steps of:
(i) providing a composition comprising compounds of formulae (I) and (II) or a pharmaceutically acceptable salt or solvate thereof, wherein R¹and R² are defined as in any one of claims 1 to 6 and wherein the amount of the compound of formula (I) present in the composition relative to the amount of the compound of formula (II) is less than about 100 ppm, less than about 75 ppm, less than about 50 ppm, less than about 25 ppm, or less than about 10 ppm; and
(ii) reacting the composition of (i) with an alkylating agent to form naltrexone or a pharmaceutically acceptable salt or solvate thereof, wherein the amount of 7,8-didehydronaltrexone present relative to the amount of naltrexone is less than about 100 ppm, less than about 75 ppm, less than about 50 ppm, less than about 25 ppm, or less than about 10 ppm.

169. The process of claim 168, wherein the alkylating agent is selected from the group consisting of cyclopropylmethyl halides and is preferably cyclopropylmethyl bromide.

170. The process of claim 168 or 169, wherein the amount of 7,8-didehydronaltrexone present is determined by the HPLC method of Example 1.4.

171. The process of any one of claims 168 to 170, wherein the amount of the compound of formula (I) present in the composition relative to the amount of the compound of formula (II) is from about 10 ppm to less than about 100 ppm, from about 10 ppm to less than about 75 ppm, from about 10 ppm to less than about 50 ppm, from about 10 ppm to less than about 40 ppm,or from about 10 ppm to less than about 25 ppm, each as determined by the HPLC method of Example 1.4.

172. Use of a composition comprising compounds of formulae (I) and (II) or a pharmaceutically acceptable salt or solvate thereof of any one of claims 117 to 171, as an intermediate or starting material for preparing a first morphinan derivative or a pharmaceutically acceptable salt or solvate thereof.

173. Use of a composition comprising compounds of formulae (I) and (II) or a pharmaceutically acceptable salt or solvate thereof of any one of claims 117 to 171, for preparing a medicament containing the composition comprising compounds of formulae (I) and (II) or the pharmaceutically acceptable salt or solvate thereof, wherein the medicament optionally comprises at least one second morphinan derivative or a pharmaceutically acceptable salt or solvate thereof.

174. Use of a composition comprising compounds of formulae (I) and (II) or a pharmaceutically acceptable salt or solvate thereof of any one of claims 117 to 171, for preparing a medicament containing the composition comprising compounds of formulae (I) and (II) or the pharmaceutically acceptable salt or solvate thereof and a first morphinan derivative or a pharmaceutically acceptable salt or solvate thereof, wherein the medicament optionally comprises at least one second morphinan derivative or pharmaceutically acceptable salt or solvate thereof.

175. The use of claims 172 to 174, wherein the first morphinan derivative is naloxone or a pharmaceutically acceptable salt or solvate thereof.

176. The use of any one of claims 172 to and 175, wherein the first morphinan derivative is naloxone hydrochloride.

177. The use of any one of claims 172 to 176, wherein the compound of formula (I) is 14-hydroxynormorphinone and the compound of formula (II) is noroxymorphone, in the synthesis of naloxone, wherein the composition comprising compounds of formulae (I) and (II) or a salt or a solvate thereof is reacted with an alkylating agent to form naloxone or a salt or a solvate thereof.

178. The use of any one of claims 172 to 177, wherein the amount present of 7,8-didehydronaloxone or a salt or a solvate thereof relative to the amount of naloxone or a salt or a solvate thereof in the product is less than about 100 ppm, less than about 75 ppm, less than about 50 ppm, less than about 25 ppm, or less than about 10 ppm, preferably each as determined by the HPLC method of Example 1.3.

179. The use of any one of claims 172 to 174, wherein the compound of formula (I) is 14-hydroxynormorphinone and the compound of formula (II) is noroxymorphone, in the synthesis of naltrexone, and wherein the composition comprising compounds of formulae (I) and (II) or a salt or a solvate thereof is reacted with an alkylating agent to form naltrexone or a salt or a solvate thereof.

180. The use of claim 172 or 179, wherein the first morphinan derivative is naltrexone or a salt or a solvate thereof.

181. The use of any one of claims 172, 174, 179, and 180, wherein the first morphinan derivative is naltrexone hydrochloride.

182. The use of any one of claims 172 to 174 and 179 to 181, wherein the amount present of 7,8-didehydronaltrexone or a salt or a solvate thereof relative to the amount of naltrexone or a salt or a solvate thereof in the product is less than about 100 ppm, less than about 75 ppm, less than about 50 ppm, less than about 25 ppm, or less than about 10 ppm, preferably each as determined by the HPLC method of Example 1.4.

183. The use of any one of claims 172 to 174, 179 and 181, wherein the amount present of 7,8-didehydronaltrexone or a salt or a solvate thereof relative to the amount of naltrexone or a salt or a solvate thereof in the product is from about 10 ppm to less than about 100 ppm, from about 10 ppm to less than about 75 ppm, from about 10 ppm to less than about 50 ppm, or from about 10 ppm to less than about 25 ppm, preferably each as determined by the HPLC method of Example 1.4.

184. The use of any one of claims 172 to 178, wherein the amount present of 7,8-didehydronaloxone or a salt or a solvate thereof relative to the amount of naloxone or a salt or a solvate thereof in the product is from about 10 ppm to less than about 100 ppm, from about 10 ppm to less than about 75 ppm, from about 10 ppm to less than about 50 ppm or from about 10 ppm to less than about 25 ppm, preferably each as determined by the HPLC method of Example 1.3.

185. A composition comprising compounds of formulae (I) and (II) or a pharmaceutically acceptable salt or solvate thereof of any one of claims 117 to 163 for use as a medicament.

186. The composition comprising compounds of formulae (I) and (II) or a pharmaceutically acceptable salt or solvate thereof for use of claim 185, wherein the composition comprising compounds of formulae (I) and (II) or the pharmaceutically acceptable salt or solvate thereof is combined with at least one other morphinan derivative or pharmaceutically acceptable salt or solvate thereof in the medicament.

187. The composition of claim 185 or 186, wherein the compound of formula (II) is naloxone or a pharmaceutically acceptable salt or solvate thereof and the compound of formula (I) is 7,8-didehydronaloxone or a pharmaceutically acceptable salt or solvate thereof.

188. The composition of claim 185 or 186, wherein the compound of formula (II) is naltrexone or a pharmaceutically acceptable salt or solvate thereof and the compound of formula (I) is 7,8-didehydronaltrexone or a pharmaceutically acceptable salt or solvate thereof.

189. The composition comprising compounds of formulae (I) and (II) or a pharmaceutically acceptable salt or solvate thereof of claim 187 or 188 for use in the treatment of opioid receptor agonist-induced bowel dysfunction, constipation, opioid receptor agonist-induced depression, opioid receptor agonist-induced overdose, or opioid receptor agonist abuse.

190. The composition comprising compounds of formulae (I) and (II) or a pharmaceutically acceptable salt or solvate thereof of claim 188 for use in the treatment of addiction.

191. A method for treating or preventing a medical condition in an animal, comprising administering to an animal in need thereof an effective amount of a composition comprising compounds of formulae (I) and (II) or pharmaceutically acceptable salts or solvates thereof of any one of claims 117 to 163.

192. The method of claim 191, wherein the compound of formula (II) is naloxone or a pharmaceutically acceptable salt or solvate thereof and the compound of formula (I) is 7,8-didehydronaloxone or a pharmaceutically acceptable salt or solvate thereof.

193. The method of claim 191, wherein the compound of formula (II) is naltrexone or a pharmaceutically acceptable salt or solvate thereof and the compound of formula (I) is 7,8-didehydronaltrexone or a pharmaceutically acceptable salt or solvate thereof.

194. The method of claim 192 or 193, wherein the medical condition is selected from the group consisting of opioid receptor agonist-induced bowel dysfunction, constipation, opioid receptor agonist-induced depression, opioid receptor agonist-induced overdose, and opioid receptor agonist abuse

195. The method of claim 193, wherein the medical condition is addiction.

196. A method for treating or preventing pain in an animal, comprising administering to an animal in need thereof an effective amount of a composition comprising compounds of formulae (I) and (II) or a pharmaceutically acceptable salt or solvate thereof of any one of claims 117 to 163.

197. The method of any one of claims 191 to 196, wherein the animal is a mammal.

198. The method of any one of claims 191 to 197, wherein the animal is a human.

199. The method of any one of claims 191 to 198, wherein the composition comprising compounds of formulae (I) and (II) or a pharmaceutically acceptable salt or solvate thereof of any one of claims 119 to 169 further comprises at least one other morphinan derivative or pharmaceutically acceptable salt or solvate thereof.
